(19) United States
(12) Patent Application Publication
(10) Pub. No.: US 2024/0390270 A1
(43) Pub. Date: Nov. 28, 2024

(54) GASTRIC RESIDENCE SYSTEMS FOR ADMINISTRATION OF RISPERIDONE

(71) Applicant: Lyndra Therapeutics, Inc., Watertown, MA (US)

(72) Inventors: Rosemary KANASTY, Cambridge, MA (US); Tyler GRANT, Arlington, MA (US); David ALTREUTER, Wayland, MA (US); Alisha WEIGHT, Cambridge, MA (US); Saumya MOORTHY, Medford, MA (US); Marlene SCHWARZ, Auburndale, MA (US); Jie JING, Watertown, MA (US); David C. DUFOUR, Watertown, MA (US); Erik Robert Waldemar RYDE, Boston, MA (US); Nupura BHISE, Watertown, MA (US); Craig SIMSES, Quincy, MA (US); Erick PEEKE, Cambridge, MA (US); Erica LAI, Somerville, MA (US); Tammy TAI, Cambridge, MA (US); Juan Jaramillo MONTEZCO, Cambridge, MA (US); Luigi ANNESE, Watertown, MA (US); Nufar HERZBERG, Cambridge, MA (US); Magali HICKEY, Westwood, MA (US); Dinara VILLANUEVA, Bedford, MA (US)

(21) Appl. No.: 18/272,786

(22) PCT Filed: Jan. 19, 2022
(86) PCT No.: PCT/US2022/013027

§ 371 Date: Jul. 17, 2023

Related U.S. Application Data
(60) Provisional application No. 63/174,497, filed on Apr. 13, 2021.
(60) Provisional application No. 63/139,299, filed on Jan. 19, 2021.

(51) Int. Cl. A61K 9/00; A61K 31/519; A61K 47/34; A61K 47/38

(52) U.S. Cl. CPC A61K 9/0065; A61K 31/519; A61K 47/34; A61K 47/38

(57) ABSTRACT

Gastric residence systems for administration of risperidone are disclosed. Features which enhance gastric retention during the desired residence time and which allow for more precise control over residence time are disclosed, including circumferential filaments connecting the arms of a stellate gastric residence system; improved time-dependent and enteric disintegrating matrices (linkers); and release rate-modulating polymer coatings which are resistant to change in release rate properties during heat-assisted assembly or thermal cycling. Combinations of these features are also disclosed.

### CROSS-REFERENCE TO RELATED APPLICATIONS

This application is a national stage application under 35 U.S.C. § 371 of International Application No. PCT/US2022/013027, filed internationally on Jan. 19, 2022, which claims priority benefit of U.S. Provisional Patent Application No. 63/139,299 filed on Jan. 19, 2021 and U.S. Provisional Patent Application No. 63/174,497 filed on Apr. 13, 2021. The entire contents of those applications are hereby incorporated by reference herein.

### STATEMENT REGARDING FEDERALLY SPONSORED RESEARCH OR DEVELOPMENT

This invention was made with government support under R01 AI131416 awarded by the National Institutes of Health. The government has certain rights in the invention.

### FIELD OF THE INVENTION

The invention relates to gastric residence systems for sustained gastric release of active agents, such as drugs, and methods of use thereof.

### BACKGROUND OF THE INVENTION

Gastric residence systems are delivery systems for agents which remain in the stomach for days to weeks, or even over longer periods, during which time drugs or other agents can elute from the systems for absorption in the gastrointestinal tract. Examples of such systems are described in U.S. Pat. No. 10,182,985, and in International Patent Application Nos. WO 2015/191920, WO 2015/191925, WO 2017/070612, WO 2017/100367, WO 2017/205844, and WO 2018/227147. Over the period of residence, the system releases an agent or agents, such as one or more drugs.

The current invention describes advancements in design, structure, and formulation of gastric residence systems, which provide improved control over residence time and release rate of agent.

### SUMMARY OF THE INVENTION

Risperidone dosage forms incorporating several features providing for more precise and consistent control of the desired residence time of gastric residence systems are disclosed. The following features are included: a filament which is wrapped circumferentially around a gastric residence system and connecting the arms of the gastric residence system; use of timed linkers and enteric linkers which permit higher precision in retention and passage of the gastric residence system; and arms coated with release rate-modulating polymer films.

The features of any of the embodiments recited above and herein are combinable with any of the other embodiments recited above and herein where appropriate and practical.

### BRIEF DESCRIPTION OF THE DRAWINGS

FIG. 1A shows a gastric residence system configuration.

FIG. 1B shows a detailed view of a gastric residence configuration.

FIG. 2A shows a configuration of a gastric residence system dosage form for risperidone.

FIG. 2B shows another configuration of a gastric residence system dosage form for risperidone.

FIG. 3 depicts a graph of the pharmacokinetics of the risperidone formulation of the gastric residence systems in Example 1 (upper curve: 28 mg dosage form; lower curve: 14 mg dosage form) in human subjects.

FIG. 4 depicts pharmacokinetics of risperidone in patients as they are transitioned from steady-state on immediate release (IR) risperidone to the extended release (ER) risperidone gastric residence systems. Concentrations of active moiety (risperidone and 9-hydroxyrisperidone combined) are plotted. The upper curve shows concentrations from administration of the 28 mg ER gastric residence systems, while the lower curve shows concentrations from administration of the 14 mg ER gastric residence systems. Bands showing C_{avg} and Cₘᵢₙ for the corresponding matched IR groups are overlaid on the curves.

FIG. 5A depicts risperidone pharmacokinetics of gastric residence system (ER) doses. The mean concentration of active moiety (risperidone and 9-hydroxyrisperidone combined) is plotted, +/−standard deviation. The top graph shows 14 mg ER doses, while the bottom graph shows 28 mg ER doses. Bands showing C_{avg} and Cₘᵢₙ for the final day of the IR lead-in are overlaid on the graphs.

FIG. 5B depicts concentration of active moiety (risperidone and 9-hydroxyrisperidone combined) of 2 mg (top graph) and 4 mg (bottom graph) risperidone daily IR administration. Bands showing C_{avg} and Cₘᵢₙ for the final day of the IR lead-in are overlaid on the graphs.

FIG. 6A depicts a comparison of C_{avg}, average concentration of active moiety (risperidone and 9-hydroxyrisperidone combined), at Day −1 (i.e., just before transition from IR risperidone to ER risperidone gastric residence systems) and at Day 15, for 2 mg IR vs. 14 mg ER and 4 mg IR vs. 28 mg ER.

FIG. 6B depicts a comparison of Cₜₐᵤ, trough concentration of active moiety (risperidone and 9-hydroxyrisperidone combined), at Day −1 (i.e., just before transition from IR risperidone to ER risperidone gastric residence systems) and at Day 15, for 2 mg IR vs. 14 mg ER and 4 mg IR vs. 28 mg ER.

FIG. 7 shows a configuration of a gastric residence system dosage form for risperidone.

FIG. 8A shows a configuration of a gastric residence system dosage form for risperidone. FIG. 8B shows another configuration of a gastric residence system dosage form for risperidone. FIG. 8C shows a configuration of a drug-eluting arm within a gastric residence system dosage form for risperidone. FIG. 8D shows an active composite arm within a gastric residence system dosage form for risperidone. FIG. 8E shows an inactive composite arm within a gastric residence system dosage form for risperidone.

FIG. 9A shows a configuration of a gastric residence system dosage form for risperidone. FIG. 9B shows a configuration of a drug-eluting arm (with active arm) within a gastric residence system dosage form for risperidone. FIG. 9C shows a configuration of a non-drug-eluting arm (with inactive arm) within a gastric residence system dosage form for risperidone.

FIG. 10A shows a configuration of a gastric residence system dosage form for risperidone. FIG. 10B shows a configuration of a drug-eluting arm (with active arm) within a gastric residence system dosage form for risperidone. FIG. 10C shows a configuration of a non-drug-eluting arm (with inactive arm) within a gastric residence system dosage form for risperidone.

FIG. 11A shows a configuration of a gastric residence system dosage form for risperidone. FIG. 11B shows a configuration of a drug-eluting arm (with active arm) within a gastric residence system dosage form for risperidone. FIG. 11C shows a configuration of a non-drug-eluting arm (with inactive arm) within a gastric residence system dosage form for risperidone.

### DETAILED DESCRIPTION OF THE INVENTION

### Definitions

A “carrier polymer” is a polymer suitable for blending with an agent, such as a drug, for use in a gastric residence system.

An “agent” is any substance intended for therapeutic, diagnostic, or nutritional use in a patient, individual, or subject. Agents include, but are not limited to, drugs, nutrients, vitamins, and minerals.

A “dispersant” is defined as a substance which aids in the minimization of particle size of agent and the dispersal of agent particles in the carrier polymer matrix. That is, the dispersant helps minimize or prevent aggregation or flocculation of particles during fabrication of the systems. Thus, the dispersant has anti-aggregant activity and anti-flocculant activity, and helps maintain an even distribution of agent particles in the carrier polymer matrix.

An “excipient” is any substance added to a formulation of an agent that is not the agent itself. Excipients include, but are not limited to, binders, coatings, diluents, disintegrants, emulsifiers, flavorings, glidants, lubricants, and preservatives. The specific category of dispersant falls within the more general category of excipient.

An “elastic polymer” or “elastomer” is a polymer that is capable of being deformed by an applied force from its original shape for a period of time, and which then substantially returns to its original shape once the applied force is removed.

“Approximately constant plasma level” refers to a plasma level that remains within a factor of two of the average plasma level (that is, between 50% and 200% of the average plasma level) measured over the period that the gastric residence system is resident in the stomach.

“Substantially constant plasma level” refers to a plasma level that remains within plus-or-minus 25% of the average plasma level measured over the period that the gastric residence system is resident in the stomach.

“Biocompatible,” when used to describe a material or system, indicates that the material or system does not provoke an adverse reaction, or causes only minimal, tolerable adverse reactions, when in contact with an organism, such as a human. In the context of the gastric residence systems, biocompatibility is assessed in the environment of the gastrointestinal tract.

A “patient,” “individual,” or “subject” refers to a mammal, preferably a human or a domestic animal such as a dog or cat. In a most preferred embodiment, a patient, individual, or subject is a human.

The “diameter” of a particle as used herein refers to the longest dimension of a particle.

“Treating” a disease or disorder with the systems and methods disclosed herein is defined as administering one or more of the systems disclosed herein to a patient in need thereof, with or without additional agents, in order to reduce or eliminate either the disease or disorder, or one or more symptoms of the disease or disorder, or to retard the progression of the disease or disorder or of one or more symptoms of the disease or disorder, or to reduce the severity of the disease or disorder or of one or more symptoms of the disease or disorder. “Suppression” of a disease or disorder with the systems and methods disclosed herein is defined as administering one or more of the systems disclosed herein to a patient in need thereof, with or without additional agents, in order to inhibit the clinical manifestation of the disease or disorder, or to inhibit the manifestation of adverse symptoms of the disease or disorder. The distinction between treatment and suppression is that treatment occurs after adverse symptoms of the disease or disorder are manifest in a patient, while suppression occurs before adverse symptoms of the disease or disorder are manifest in a patient. Suppression may be partial, substantially total, or total. Because some diseases or disorders are inherited, genetic screening can be used to identify patients at risk of the disease or disorder. The systems and methods disclosed herein can then be used to treat asymptomatic patients at risk of developing the clinical symptoms of the disease or disorder, in order to suppress the appearance of any adverse symptoms.

“Therapeutic use” of the systems disclosed herein is defined as using one or more of the systems disclosed herein to treat a disease or disorder, as defined above. A “therapeutically effective amount” of a therapeutic agent, such as a drug, is an amount of the agent, which, when administered to a patient, is sufficient to reduce or eliminate either a disease or disorder or one or more symptoms of a disease or disorder, or to retard the progression of a disease or disorder or of one or more symptoms of a disease or disorder, or to reduce the severity of a disease or disorder or of one or more symptoms of a disease or disorder. A therapeutically effective amount can be administered to a patient as a single dose, or can be divided and administered as multiple doses.

“Prophylactic use” of the systems disclosed herein is defined as using one or more of the systems disclosed herein to suppress a disease or disorder, as defined above. A “prophylactically effective amount” of an agent is an amount of the agent, which, when administered to a patient, is sufficient to suppress the clinical manifestation of a disease or disorder, or to suppress the manifestation of adverse symptoms of a disease or disorder. A prophylactically effective amount can be administered to a patient as a single dose, or can be divided and administered as multiple doses.

A “flexural modulus” of a material is an intrinsic property of a material computed as the ratio of stress to strain in flexural deformation of the material as measured by a 3-point bending test. Although the linkers are described herein as being components of the gastric residence system, the flexural modulus of the material of the polymeric material may be measured in isolation. For example, the polymeric linker in the gastric residence system may be too short to measure the flexural modulus, but a longer sample of the same material may be used to accurately determine the flexural modulus. The longer sample used to measure the flexural modulus should have the same cross-sectional dimensions (shape and size) as the polymeric linker used in the gastric residence system. The flexural modulus is measured using a 3-point bending test in accordance with the ASTM standard 3-point bending test (ASTM D790) using a 10 mm distance between supports and further modified to accommodate materials with non-rectangular cross-sections. The longest line of symmetry for the cross section of the polymeric linker should be positioned vertically, and the flexural modulus should be measured by applying force downward. If the longest line of symmetry for the cross section of the polymeric linker is perpendicular to a single flat edge, the single flat edge should be positioned upward. If the cross-section of the polymeric linker is triangular, the apex of the triangle should be faced downward. As force is applied downward, force and displacement are measured, and the slope at the linear region is obtained to calculate the flexural modulus.

The concentration of risperidone active moiety is the concentration of risperidone plus the concentration of 9-hydroxyrisperidone. Concentration of risperidone active moiety is typically measured in blood plasma.

As used herein, the singular forms “a”, “an”, and “the” include plural references unless indicated otherwise or the context clearly dictates otherwise.

When numerical values are expressed herein using the term “about” or the term “approximately,” it is understood that both the value specified, as well as values reasonably close to the value specified, are included. For example, the description “about 50° C.” or “approximately 50° C.” includes both the disclosure of 50° C. itself, as well as values close to 50° C. Thus, the phrases “about X” or “approximately X” include a description of the value X itself. If a range is indicated, such as “approximately 50° C. to 60° C.” or “about 50° C. to 60° C.,” it is understood that both the values specified by the endpoints are included, and that values close to each endpoint or both endpoints are included for each endpoint or both endpoints; that is, “approximately 50° C. to 60° C.” (or “about 50° C. to 60° C.”) is equivalent to reciting both “⁴50° C. to 60° C.” and “approximately 50° C. to approximately 60° C.” (or “about 50° C. to 60° C.”).

With respect to numerical ranges disclosed in the present description, any disclosed upper limit for a component may be combined with any disclosed lower limit for that component to provide a range (provided that the upper limit is greater than the lower limit with which it is to be combined). Each of these combinations of disclosed upper and lower limits are explicitly envisaged herein. For example, if ranges for the amount of a particular component are given as 10% to 30%, 10% to 12%, and 15% to 20%, the ranges 10% to 20% and 15% to 30% are also envisaged, whereas the combination of a 15% lower limit and a 12% upper limit is not possible and hence is not envisaged.

Unless otherwise specified, percentages of ingredients in compositions are expressed as weight percent, or weight/weight percent. It is understood that reference to relative weight percentages in a composition assumes that the combined total weight percentages of all components in the composition add up to 100. It is further understood that relative weight percentages of one or more components may be adjusted upwards or downwards such that the weight percent of the components in the composition combine to a total of 100, provided that the weight percent of any particular component does not fall outside the limits of the range specified for that component.

Some embodiments described herein are recited as “comprising” or “comprises” with respect to their various elements. In alternative embodiments, those elements can be recited with the transitional phrase “consisting essentially of” or “consists essentially of” as applied to those elements. In further alternative embodiments, those elements can be recited with the transitional phrase “consisting of” or “consists of” as applied to those elements. Thus, for example, if a composition or method is disclosed herein as comprising A and B, the alternative embodiment for that composition or method of “consisting essentially of A and B” and the alternative embodiment for that composition or method of “consisting of A and B” are also considered to have been disclosed herein. Likewise, embodiments recited as “consisting essentially of” or “consisting of” with respect to their various elements can also be recited as “comprising” as applied to those elements. Finally, embodiments recited as “consisting essentially of” with respect to their various elements can also be recited as “consisting of” as applied to those elements, and embodiments recited as “consisting of” with respect to their various elements can also be recited as “consisting essentially of” as applied to those elements.

When a composition or system is described as “consisting essentially of” the listed elements, the composition or system contains the elements expressly listed, and may contain other elements which do not materially affect the condition being treated (for compositions for treating conditions), or the properties of the described system (for compositions comprising a system). However, the composition or system either does not contain any other elements which do materially affect the condition being treated other than those elements expressly listed (for compositions for treating systems) or does not contain any other elements which do materially affect the properties of the system (for compositions comprising a system); or, if the composition or system does contain extra elements other than those listed which may materially affect the condition being treated or the properties of the system, the composition or system does not contain a sufficient concentration or amount of those extra elements to materially affect the condition being treated or the properties of the system. When a method is described as “consisting essentially of” the listed steps, the method contains the steps listed, and may contain other steps that do not materially affect the condition being treated by the method or the properties of the system produced by the method, but the method does not contain any other steps which materially affect the condition being treated or the system produced other than those steps expressly listed.

This disclosure provides several embodiments. It is contemplated that any features from any embodiment can be combined with any features from any other embodiment where possible. In this fashion, hybrid configurations of the disclosed features are within the scope of the present disclosure.

In addition to the embodiments and methods disclosed here, additional embodiments of gastric residence systems, and methods of making and using such systems, are disclosed in International Patent Application Nos. WO 2015/191920, WO 2015/191925, WO 2017/070612, WO 2017/100367, and PCT/US2017/034856, which are incorporated by reference herein in their entirety.

The following abbreviations for polymers and other components are used:

| Abbreviation | Component |
|---|---|
| PDL | poly(DL-lactide); inherent viscosity 1.6-2.4 dl/g |
| | (CHCl₃), Tₘ 165-180° C. |
| PDL20 | GMP grade copolymer of DL-lactide with an |
| | inherent viscosity midpoint of 2.0 dl/g (such |
| | as PURASORB ® Poly-D,L-lactide) |
| PCL HMW | polycaprolactone; MW(ave) 200,000 |
| PCL LMW | polycaprolactone; MW (ave) 15,000 |
| VA64 | copovidone; Tₘ 140° C., T_{g} 101° C. |
| Kollidon SR | Polyvinyl acetate/polyvinylpyrrolidone |
| K90F | povidone; T_{g} 156° C. |
| PEG1 | polyethylene glycol; MW (ave) 1,000 |
| PEO_{100K} | polyethylene glycol; MW (ave) 100,000 |
| L-31 | PEG-PPG-PEG block co-polymer; MW (ave) |
| | 1,100 (Mₙ) (such as Pluronic ® L-31) |
| PPG | polypropylene glycol |
| PDLG | copolymer of DL-lactide and glycolide); inherent |
| | viscosity 1.6-2.4 dl/g (CHCl₃) |
| PCL triol | polycaprolactone triol; MW (ave) 900 (Mₙ) |
| Corbion PC17 | PURASORB ® Polycaprolactone; GMP grade |
| | homopolymer of ε-Caprolactone with an inherent |
| | viscosity midpoint of 1.7 dl/g |
| Corbion PC12 | PURASORB ® Polycaprolactone; GMP grade |
| | homopolymer of ε-Caprolactone with an inherent |
| | viscosity midpoint of 1.2 dl/g |
| Corbion PC04 | PURASORB ® Polycaprolactone; GMP grade |
| | homopolymer of ε-Caprolactone with an inherent |
| | viscosity midpoint of 0.4 dl/g |
| F-108 | Pluronic ® F-108; PEG-PPG-PEG block co-polymer |
| PDL-PCL | poly D-lactide-polycaprolactone co-polymer |
| 25-75 | |
| PDL-PCL | poly D-lactide-polycaprolactone co-polymer |
| 80-20 | |
| PG | propylene glycol |
| PVPP | crospovidone |
| PVAc | polyvinylacetate |
| PEG10 | polyethylene glycol; MW (ave) 10,000 |
| Corbion 5004 | PURASORB ® 50/50 DL-lactide/glycolide copolymer; |
| (PDLG5004) | ester-terminated (such as ethylester-terminated or |
| | methylester-terminated) GMP grade copolymer of DL- |
| | lactide and Glycolide in a 50/50 molar ratio and |
| | with an inherent viscosity midpoint of 0.4 dl/g |
| Corbion 5004A | PURASORB ® 50/50 DL-lactide/glycolide copolymer; |
| (PDLG5004A) | acid terminated (such as carboxylic acid-terminated) |
| | GMP grade copolymer of DL-lactide and Glycolide in |
| | a 50/50 molar ratio and with an inherent viscosity |
| | midpoint of 0.4 dl/g |
| Corbion 5002A | PURASORB ® 50/50 DL-lactide/glycolide copolymer; |
| (PDLG5002A) | acid terminated (such as carboxylic acid-terminated) |
| | GMP grade copolymer of DL-lactide and Glycolide in |
| | a 50/50 molar ratio and with an inherent viscosity |
| | midpoint of 0.2 dl/g |
| HPMCAS | Synthetic polymer derived from cellulose |
| P407 | Poloxamer 407; PEG-PPG-PEG triblock co-polymer |
| E172 | Ferrosoferric Oxide |
| Span60 | Sorbitan monostearate |

PLURONIC® is a registered trademark of BASE Corporation for polyoxyalkylene ethers. In any formulation described herein using trade names, the trade name can be replaced by the generic name. For example, a formulation described as comprising 50% Corbion PC 17 and 50% Corbion PC04 is understood to describe a formulation comprising 50% polycaprolactone of viscosity 1.7 dl/g and 50% polycaprolactone of viscosity 0.4 dl/g. Any component in any formulation described herein using a trade name can be replaced with an equivalent component from another manufacturer.

As used herein, unless otherwise specified, a “copolymer of DL-lactide and glycolide” is understood to refer to an ester-terminated copolymer of DL-lactide and glycolide; and a “poly(D,L-lactic-co-glycolide)” is understood to refer to an ester-terminated poly(D,L-lactic-co-glycolide).

As used herein, unless otherwise specified, “PCL” can refer to polycaprolactone with various inherent viscosity midpoints, such as from 1.0 to 2.1 dl/g, such as polycaprolactone with an inherent viscosity midpoint of 1.7 dl/g or polycaprolactone with an inherent viscosity midpoint of 1.2 dl/g.

### Gastric Residence System Description

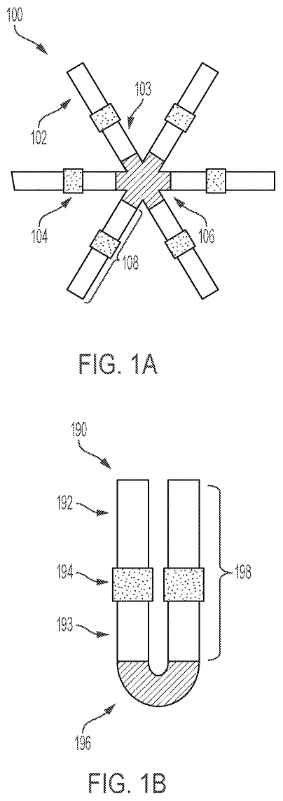

Gastric residence systems can be prepared in different configurations. The “stellate” configuration of a gastric residence system is also known as a “star” (or “asterisk”) configuration. An example of a stellate system 100 is shown schematically in FIG. 1A. Multiple arms (only one such arm, 108, is labeled for clarity), are affixed to disk-shaped central elastomer 106. The arms depicted in FIG. 1A are comprised of segments 102 and 103, joined by a coupling polymer or linker region 104 (again, the components are only labeled in one arm for clarity) which serves as a linker region. This configuration permits the system to be folded or compacted at the central elastomer. FIG. 1B shows a folded configuration 190 of the gastric residence system of FIG. 1A (for clarity, only two arms are illustrated in FIG. 1B). Segments 192 and 193, linker region 194, elastomer 196, and arm 198 of FIG. 1B correspond to segments 102 and 103, linker region 104, elastomer 106, and arm 108 of FIG. 1A, respectively. When folded, the overall length of the system is reduced by approximately a factor of two, and the system can be conveniently placed in a container such as a capsule or other container suitable for oral administration. The gastric residence system is constrained by the capsule or other container into the compacted state (the folded state). When the capsule reaches the stomach, the capsule dissolves, releasing the gastric residence system. Upon release of the constraint by the capsule or other container, the gastric residence system then unfolds into its uncompacted state, which is retained in the stomach for the desired residence period.

While the linker regions 104 are shown as slightly larger in diameter than the segments 102 and 103 in FIG. 1A, they can be the same diameter as the segments, so that the entire arm 102-104-103 has a smooth outer surface.

In some embodiments, the stellate system may have an arm composed of only one segment, which is attached to the central elastomer by a linker region. This corresponds to FIG. 1A with the segments 103 omitted. The single-segment arms comprising segments 102 are then directly attached to central elastomer 106 via the linkers 104. The linkers can comprise a coupling polymer or a disintegrating matrix.

A stellate system can be described as a gastric residence system for administration to the stomach of a patient, comprising an elastomer component, and a plurality of at least three carrier polymer-agent components comprising a carrier polymer and an agent or a salt thereof, attached to the elastomer component, wherein each of the plurality of carrier polymer-agent components is an arm comprising a proximal end, a distal end, and an outer surface therebetween; wherein the proximal end of each arm is attached to the elastomer component and projects radially from the elastomer component, each arm having its distal end not attached to the elastomer component and located at a larger radial distance from the elastomer component than the proximal end; wherein each arm independently comprises one or more segments, each segment comprising a proximal end, a distal end, and an outer surface therebetween. In some embodiments, when two or more segments are present in an arm, each segment is attached to an adjacent segment via a linker region. In some embodiments, when two or more segments are present in an arm, one segment is directly attached to the other segment, without using a linker region. The linker region can be a coupling polymer or a disintegrating matrix. The arms can be attached to the central elastomer via a coupling polymer or a disintegrating matrix, and can have intervening portions of interfacing polymers. For the plurality of at least three arms, or for a plurality of arms, a preferred number of arms is six, but three, four, five, seven, eight, nine, or ten arms can be used. The arms should be equally spaced around the central elastomer; if there are N arms, there will be an angle of about 360/N degrees between neighboring arms.

The coupling polymers of the gastric residence system, which serve as linker regions, are designed to break down gradually in a controlled manner during the residence period of the system in the stomach. If the gastric residence system passes prematurely into the small intestine in an intact form, the system is designed to break down much more rapidly to avoid intestinal obstruction. This is readily accomplished by using enteric polymers as coupling polymers. Enteric polymers are relatively resistant to the acidic pH levels encountered in the stomach, but dissolve at the higher pH levels found in the duodenum. Use of enteric coupling polymers as safety elements protects against undesired passage of the intact gastric residence system into the small intestine. In the system shown in FIG. 1A, at least the coupling polymer used for the couplings 104 are made from such enteric polymers.

In additional embodiments, a time-dependent coupling polymer or linker can be used. Such a time-dependent coupling polymer or linker degrades in a predictable, time-dependent manner. In some embodiments, the degradation of the time-dependent coupling polymer or linker may not be affected by the varying pH of the gastrointestinal system.

In additional embodiments, different types of linkers can be used in the gastric residence systems. That is, both enteric linkers (or enteric coupling polymers) and time-dependent linkers (or time-dependent coupling polymers) can be used. In some embodiments, a single multi-segment arm of a stellate system can use both an enteric linker at some linker regions between segments, and a time-dependent linker at other linker regions between segments.

Linker regions are typically about 100 microns to about 2 millimeter in width, such as about 200 μm to about 2000 μm, about 300 μm to about 2000 μm, about 400 μm to about 2000 μm, about 500 μm to about 2000 μm, about 600 μm to about 2000 μm, about 700 μm to about 2000 μm, about 800 μm to about 2000 μm, about 900 μm to about 2000 μm, about 1000 μm to about 2000 μm, about 1100 μm to about 2000 μm, about 1200 μm to about 2000 μm, about 1300 μm to about 2000 μm, about 1400 μm to about 2000 μm, about 1500 μm to about 2000 μm, about 1600 μm to about 2000 μm, about 1700 μm to about 2000 μm, about 1800 μm to about 2000 μm, or about 1900 μm to about 2000 μm; or about 100 μm to about 1900 μm, about 100 μm to about 1800 μm, about 100 μm to about 1700 μm, about 100 μm to about 1600 μm, about 100 μm to about 1500 μm, about 100 μm to about 1400 μm, about 100 to about 1300 μm, about 100 μm to about 1200 μm, about 100 μm to about 1100 μm, about 100 μm to about 1000 μm, about 100 μm to about 900 μm, about 100 μm to about 800 μm, about 100 μm to about 700 μm, about 100 μm to about 600 μm, about 100 μm to about 500 μm, about 100 μm to about 400 μm, about 100 μm to about 300 μm, or about 100 μm to about 200 um. Linker regions can be about 100 μm, about 200 μm, about 300 μm, about 400 μm, about 500 μm, about 600 μm, about 700 μm, about 800 μm, about 900 μm, about 1000 μm, about 1100 μm, about 1200 μm, about 1300 μm, about 1400 μm, about 1500 μm, about 1600 μm, about 1700 μm, about 1800 μm, about 1900 μm, or about 200 um in width, where each value can be plus or minus 50 um (±50 um).

The central elastomeric polymer of a stellate system is typically not an enteric polymer; however, the central elastomeric polymer can also be made from such an enteric polymer where desirable and practical.

The central elastomer should have a specific durometer and compression set. The durometer is important because it determines the folding force of the dosage form and whether it will remain in the stomach; a preferred range is from about 60 to about 90 A. The compression set should be as low as possible to avoid having permanent deformation of the gastric residence system when stored in the capsule in its compacted configuration. A preferred range is about 10% to about 20% range. Liquid silicone rubber is a useful material for the central elastomer. Examples of materials that fit these requirements are the QP1 range of liquid silicone rubbers from Dow Corning. In any embodiment with a central elastomer, the QP1-270 (70 A durometer) liquid silicone rubber can be used. In some embodiments, the central elastomer may comprise a 50 A or 60 A durometer liquid silicone rubber (Shin Etsu).

Segments and arms of the gastric residence systems can have cross-sections in the shape of a circle (in which case the segments are cylindrical), a polygon (such as segments with a triangular cross-section, rectangular cross-section, or square cross-section), or a pie-shaped cross-section (in which case the segments are cylindrical sections). Segments with polygon-shaped or pie-shaped cross-sections, and ends of cylindrically-shaped sections which will come into contact with gastric tissue, can have their sharp edges rounded off to provide rounded corners and edges, for enhanced safety in vivo. That is, instead of having a sharp transition between intersecting edges or planes, an arc is used to transition from one edge or plane to another edge or plane. Thus, a “triangular cross-section” includes cross-sections with an approximately triangular shape, such as a triangle with rounded corners. An arm with a triangular cross-section includes an arm where the edges are rounded, and the corners at the end of the arm are rounded. Rounded corners and edges are also referred to as fillet corners, filleted corners, fillet edges, or filleted edges.

In some embodiments, the stellate system is about 30 mm to about 60 mm when unfolded (arm extended). In some embodiments, the stellate system is about 41 mm to about 51 mm when unfolded. In some embodiments, the stellate system is about 45 mm to about 47 mm when unfolded. In some embodiments, the stellate system is about 46 mm when unfolded.

### Features for Improved Retention and Agent Release for Risperidone Gastric Residence Systems

Retention of gastric residence systems for the desired residence period and agent release from gastric residence systems can be improved and made more consistent using the features described herein, such as a filament which is wrapped circumferentially around a gastric residence system and connecting the arms of the gastric residence system; use of timed linkers and enteric linkers which permit higher precision in retention and passage of the gastric residence system; and arms coated with release rate-modulating polymer films.

### Circumferential Filament

Provided in this Circumferential Filament disclosure are gastric residence systems comprising a filament for improved gastric residence and methods of preparing gastric residence forms having a filament. In particular, gastric residence systems having a filament described herein may help improve the gastric residence of the gastric residence system. Specifically, a filament can help provide a more consistent gastric residence time and/or a longer gastric residence time. Thus, gastric residence systems provided herein that include a filament may provide more predictable and/or controllable gastric residence times. Gastric residence systems having predictable and/or controllable gastric residence times can minimize the risk of the gastric residence system unfolding too early (e.g., in the esophagus) and causing an obstruction. Gastric residence systems having predictable and/or controllable gastric residence times can also minimize the possibility of the gastric residence system passing through the stomach and unfolding later in the gastrointestinal tract (i.e., intestine), or passing through the gastrointestinal tract without unfolding at all. In each of these possible scenarios, the therapeutic agent of the gastric residence dosage form is not delivered to the patient as intended.

However, it has been demonstrated that gastric residence systems of a stellate shape can bend into a configuration that allows for premature passage through the pylorus of a patient. Gastric residence systems that prematurely pass through the pylorus fail to deliver the therapeutic agent of the gastric residence system to the patient. Further, premature passage causes inconsistency, causes unreliability, and compromises the efficacy of the gastric residence system.

The feature of circumferential filament is described in International Patent Application PCT/US2020/059541, which is hereby incorporated by reference in its entirety.

In some embodiments, the filament is a non-disintegrating filament. In some embodiments, the filament comprises thermoplastic polyurethane, such as Pellethane 80 A. In some embodiments, the filament comprises methylene bis(4-phenylisocyanate), poly(tetramethylene oxide), and 1,4-butanediol. In some embodiments, the filament is a disintegrating filament. In some embodiments, the filament comprises poly (lactic-co-glycolic acid). In some embodiments, the filament comprises polyglycolic acid. In some embodiments, the thickness of the filament is about any one of 0.05 mm, 0.1 mm, 0.15 mm, 0.20 mm, 0.25 mm, 0.30 mm, 0.35 mm, 0.40 mm, 0.45 mm, 0.5 mm, 0.6 mm, 0.7 mm, 0.8 mm, 0.9 mm, 1.0 mm or any thickness therebetween. In some embodiments, the thickness of the filament is about 0.20 mm. In some embodiments, the thickness of the filament is about 0.30 mm.

In some embodiments, each section of filament connecting two adjacent arms can be about 20 to about 25 mm in length, such as about 21 to about 24 mm in length, such as about 22.8 mm. In some embodiments, the circumferential filament can be about 95 to about 120 mm in total length, such as about 100 to about 110 mm in total length, such as about 105 mm in total length.

### Timed Linkers (Timed Disintegrating Matrices) and Enteric Linkers (Enteric Disintegrating Matrices)

### Polymeric Linkers

The agent-containing structural members are attached to a second structural member (such as a central member, which may be an elastic central member) through one or more linkers. A polymeric linker may directly interface with the agent-containing structural member, or may interface with the agent-containing structural member through a coupling member. Similarly, the polymeric linker may interface directly with the second structural member, or may interface through a coupling member. In an embodiment wherein the agent-containing structural member is connected to the second structural member through two or more polymeric linkers, the polymeric linkers may directly interface with each other, or may interface through a coupling member. One or both of an enteric linker and a time-dependent linkers may be used, or a polymeric linker may function as both an enteric linker and a time-dependent linker.

The polymeric linkers are typically about 100 microns to about 3 millimeter in width, such as about 200 μm to about 3000 μm, about 300 μm to about 3000 μm, about 400 μm to about 3000 μm, about 500 μm to about 3000 μm, about 600 μm to about 3000 μm, about 700 μm to about 3000 μm, about 800 μm to about 3000 μm, about 900 μm to about 3000 μm, about 1000 μm to about 3000 μm, about 1100 μm to about 3000 μm, about 1200 μm to about 3000 μm, about 1300 μm to about 3000 μm, about 1400 μm to about 3000 μm, about 1500 μm to about 3000 μm, about 1600 μm to about 3000 μm, about 1700 μm to about 3000 μm, about 1800 μm to about 3000 μm, about 1900 μm to about 3000 μm, about 2000 μm to about 3000 μm, about 2100 um to about 3000 μm, about 2200 μm to about 3000 μm, about 2300 μm to about 3000 μm, about 2400 μm to about 3000 μm, about 2500 μm to about 3000 μm, about 2600 μm to about 3000 μm, about 2700 μm to about 3000 μm, about 2800 μm to about 3000 μm, or about 2900 μm to about 3000 μm; or about 100 μm to about 200 μm, about 200 μm to about 300 μm, about 300 μm to about 400 μm, about 400 μm to about 500 μm, about 500 μm to about 600 μm, about 600 μm to about 700 μm, about 700 μm to about 800 μm, about 800 μm to about 900 μm, about 900 μm to about 1000 μm, about 1000 μm to about 1100 μm, about 1100 μm to about 1200 μm, about 1200 μm to about 1300 μm, about 1300 μm to about 1400 μm, about 1400 μm to about 1500 μm, about 1500 μm to about 1600 μm, about 1600 μm to about 1700 μm, about 1700 μm to about 1800 μm, about 1800 μm to about 1900 μm, about 1900 μm to about 2000 μm, about 2000 μm to about 2100 μm, about 2100 μm to about 2200 μm, about 2200 μm to about 2300 μm, about 2300 μm to about 2400 μm, about 2400 μm to about 2500 μm, about 2500 μm to about 2600 μm, about 2600 μm to about 2700 μm, about 2700 μm to about 2800 μm, about 2800 μm to about 2900 μm, about 2900 μm to about 3000 um. Polymeric linkers can be about 100 μm, about 200 μm, about 300 μm, about 400 μm, about 500 μm, about 600 μm, about 700 μm, about 800 μm, about 900 μm, about 1000 μm, about 1100 μm, about 1200 μm, about 1300 μm, about 1400 μm, about 1500 μm, about 1600 μm, about 1700 μm, about 1800 μm, about 1900 μm, about 2000 μm, about 2100 μm, about 2200 μm, about 2300 μm, about 2400 μm, about 2500 μm, about 2600 μm, about 2700 μm, about 2800 μm, about 2900 μm, about 3000 μm in width, where each value can be plus or minus 50 um (±50 um).

The cross section of the polymeric linker may be round (i.e., circular), elliptical, triangular, square, rectangular, pentagonal, hexagonal, or any other polymeric shape. In some embodiments, the cross-section of the polymeric linker is the same shape as the cross-section of an agent-containing structural member attached to the polymeric linker. In some embodiments, the cross-section of the polymeric linker has a larger area than the cross-section of the agent-containing structural member, a smaller area than the cross-section of the agent-containing structural member, or approximately the same area as the cross-section of the attached agent-containing structural member.

### Time-Dependent Disintegrating Matrices (Time-Dependent Linkers)

A time-dependent linker degrades in a predictable, time-dependent manner under aqueous conditions, such as when the gastric residence system is deployed in the stomach of an individual. The time-dependent polymeric linkers control the residence time of the gastric residence system in the stomach. The time-dependent polymeric linkers are designed to degrade, dissolve, mechanically weaken, or break gradually over time. After the desired residence period, the time-dependent polymeric linker has degraded, dissolved, disassociated, or mechanically weakened, or has broken, to the point where the gastric residence system can pass through the pyloric valve, exiting the gastric environment and entering the small intestine, for eventual elimination from the body.

The time-dependent polymeric linker preferably comprises a pH-independent degradable polymer, which degrades under aqueous conditions in a pH-independent or approximately pH-independent manner. Exemplary pH-independent degradable polymer include PLGA, PLA, PCL, polydioxanone, cellulose, or blends or copolymers thereof.

The time-dependent polymeric linker can include poly(lactic-co-glycolide) (PLGA).

In some embodiments, the PLGA of the time-dependent polymeric linker comprises an ester-terminated copolymer of DL-lactide and glycolide (50/50 molar ratio) having a viscosity midpoint between about 0.32 dl/g to about 0.48 dl/g (such as about 0.4 dl/g) (such as the PLGA sold under the tradename Purasorb® PDLG 5004, available from Corbion). In some embodiments, the PLGA of the time-dependent polymeric linker comprises acid terminated copolymer of DL-lactide and glycolide (50/50 molar ratio) having a viscosity midpoint between about 0.32 dl/g to about 0.48 dl/g (such as about 0.4 dl/g) (such as the PLGA sold under the tradename Purasorb® PDLG 5004A available from Corbion). In some embodiments, the PLGA of the time-dependent polymeric linker comprises a mixture of (a) ester-terminated poly(D,L-lactic-co-glycolide) with a ratio of lactide monomers to glycolide monomers of about 50:50 (such as the PLGA sold under the tradename Purasorb® PDLG 5004, available from Corbion), and (b) acid-terminated poly(D,L-lactic-co-glycolide) with a ratio of lactide monomers to glycolide monomers of about 50:50 (such as the PLGA sold under the tradename Purasorb® PDLG 5004A, available from Corbion).

The one or more additional linker polymers included in the polymer linker is preferably homogenously mixed with the PLGA. In some embodiments, the one or more additional linker polymers are miscible with the PLGA. The one or more additional linker polymers may be a non-degradable polymer (that is, not degradable or in the gastric or enteric environment, or an aqueous solution of pH 1.6 (representing the gastric environment) or pH 6.5 (representing the enteric environment), and is optionally present in the time-dependent polymeric linker is an amount such that the time-dependent polymeric linker does not break during the gastric residence period.

Bonding of the polymeric linker to a directly adjacent member may be improved if at least one polymer is common to both the adjacent member and the time-dependent polymeric linker. In some embodiments, the at least one common polymer is polycaprolactone (PCL).

In some embodiments, the one or more additional linker polymers comprises a PCL. The time-dependent polymeric linker may be directly joined or bonded to another member of the gastric residence system (such as the structural member comprising the drug and the carrier polymer, a coupling member, the enteric polymeric linker, or a central structural member), which may also include a PCL, which may be the same PCL in the time-dependent polymeric linker or a different PCL as the one in the polymeric linker, and which may be at the same concentration or a different concentration. A different PCL in the time-dependent polymeric linker and the other member directly joined or bonded to the time-dependent linker may differ, for example, in the weight-average molecular weight of the PCL, the inherent viscosity of the PCL, or the proportions of PCL (for example, when a blend of two or more PCL polymers are used). In some embodiments, the time-dependent disintegrating matrix comprises about 40 wt % to about 50 wt % PCL. In some embodiments, the time-dependent disintegrating matrix comprises about 43 wt % to about 47 wt % PCL. In some embodiments, the time-dependent disintegrating matrix comprises about 45 wt % PCL. In some embodiments, the time-dependent disintegrating matrix comprises about 44.95 wt % PCL. In some embodiments, the time-dependent disintegrating matrix comprises about 45 wt % to about 55 wt % PCL. In some embodiments, the time-dependent disintegrating matrix comprises about 48 wt % to about 52 wt % PCL. In some embodiments, the time-dependent disintegrating matrix comprises about 50 wt % PCL. In some embodiments, the time-dependent disintegrating matrix comprises about 49.95 wt % PCL. In some embodiments, the PCL has a viscosity midpoint between about 1.5 dl/g to about 2.1 dl/g, such as about 1.7 dl/g, such as Corbion PC17. In some embodiments, the PCL has a viscosity midpoint between about 1.0 dl/g to about 1.4 dl/g, such as about 1.2 dl/g, such as Corbion PC12.

The time-dependent polymeric linker may further include one or more plasticizers, such as polyethylene glycol. The term “polyethylene glycol” is used interchangeably herein with the terms “polyethylene oxide” and “PEO.” In some embodiments, the molecular weight of the polyethylene glycol is about 90K to about 110K, such as 100 k (also referred to as 100K or 100 kDa. In some embodiments, the time-dependent disintegrating matrix comprises polyethylene glycol with molecular weight of about 100 k (polyethylene glycol 100 k). In some embodiments, the time-dependent disintegrating matrix comprises about 0.5 wt % to about 5 wt % polyethylene glycol 100 k. In some embodiments, the time-dependent disintegrating matrix comprises about 1 wt % to about 3 wt % polyethylene glycol 100 k. In some embodiments, the time-dependent disintegrating matrix comprises about 2 wt % polyethylene glycol 100 k. In some embodiments, the time-dependent disintegrating matrix comprises about 1.5 wt % to about 3.5 wt % polyethylene glycol 100 k. In some embodiments, the time-dependent disintegrating matrix comprises about 2.5 wt % polyethylene glycol 100 k. In some embodiments, the time-dependent disintegrating matrix includes a color-absorbing dyes (also referred to as a colorant or a pigment). A color-absorbing dye may be included to enhance bonding or attachment of the polymeric linker to other gastric residence system components. Color-absorbing dyes can absorb heat during the laser-welding, infrared welding, or other heat-induced attachment, which increases the tensile strength of the resulting bond. Exemplary color-absorbing dyes include iron oxide and carbon black. The time-dependent disintegrating matrix may include the color-absorbing dye in an amount of up to about 5%, such as up to about 4%, up to about 3%, up to about 2%, up to about 1%, up to about 0.5%, up to about 0.3%, up to about 0.2%, up to about 0.1%, or up to about 0.05%. In some embodiments, the time-dependent disintegrating matrix comprises about 0.005 wt % to about 0.2 wt % color-absorbing dye. In some embodiments, the time-dependent disintegrating matrix comprises about 0.01 wt % to about 0.1 wt % color-absorbing dye. In some embodiments, the time-dependent disintegrating matrix comprises about 0.05 wt % color-absorbing dye. In some embodiments, the color-absorbing dye is E172.

In one example of a time-dependent disintegrating matrix, the time-dependent disintegrating matrix comprises about 40 wt % to about 50 wt % PCL, about 30 wt % to about 40 wt % of acid terminated copolymer of DL-lactide and glycolide (50/50 molar ratio) having a viscosity midpoint of about 0.4 dl/g, about 10 wt % to about 25 wt % of copolymer of DL-lactide and glycolide (50/50 molar ratio) having a viscosity midpoint of about 0.4 dl/g, about 0.5 wt % to about 5 wt % of polyethylene glycol 100 k, and about 0.005 wt % to about 0.2 wt % color-absorbing dye E172. In one example of a time-dependent disintegrating matrix, the time-dependent disintegrating matrix comprises about 40 wt % to about 50 wt % PCL (such as PCL having a viscosity midpoint between about 1.5 dl/g to about 1.9 dl/g), about 30 wt % to about 40 wt % of acid terminated copolymer of DL-lactide and glycolide (50/50 molar ratio) having a viscosity midpoint of about 0.4 dl/g, about 10 wt % to about 25 wt % of ester terminated copolymer of DL-lactide and glycolide (50/50 molar ratio) having a viscosity midpoint of about 0.4 dl/g, about 0.5 wt % to about 5 wt % of polyethylene glycol 100 k, and about 0.005 wt % to about 0.2 wt % color-absorbing dye E172. In one example of a time-dependent disintegrating matrix, the time-dependent disintegrating matrix comprises about 40 wt % to about 50 wt % PCL (such as PCL having a viscosity midpoint between about 1.0 dl/g to about 1.4 dl/g), about 30 wt % to about 40 wt % of acid terminated copolymer of DL-lactide and glycolide (50/50 molar ratio) having a viscosity midpoint of about 0.4 dl/g, about 10 wt % to about 25 wt % of ester terminated copolymer of DL-lactide and glycolide (50/50 molar ratio) having a viscosity midpoint of about 0.4 dl/g, about 0.5 wt % to about 5 wt % of polyethylene glycol 100 k, and about 0.005 wt % to about 0.2 wt % color-absorbing dye E172. In one example of a time-dependent disintegrating matrix, the time-dependent disintegrating matrix comprises about 45 wt % to about 55 wt % PCL (such as PCL having a viscosity midpoint between about 1.0 dl/g to about 1.4 dl/g), about 27 wt % to about 37 wt % of acid terminated copolymer of DL-lactide and glycolide (50/50 molar ratio) having a viscosity midpoint of about 0.4 dl/g, about 12 wt % to about 22 wt % of ester terminated copolymer of DL-lactide and glycolide (50/50 molar ratio) having a viscosity midpoint of about 0.4 dl/g, about 0.5 wt % to about 5 wt % of polyethylene glycol 100 k, and about 0.005 wt % to about 0.2 wt % color-absorbing dye E172. In one example of a time-dependent disintegrating matrix, the time-dependent disintegrating matrix comprises about 45 wt % to about 55 wt % PCL (such as PCL having a viscosity midpoint between about 1.0 dl/g to about 1.4 dl/g), about 33 wt % to about 43 wt % of acid terminated copolymer of DL-lactide and glycolide (50/50 molar ratio) having a viscosity midpoint of about 0.4 dl/g, about 5 wt % to about 15 wt % of ester terminated copolymer of DL-lactide and glycolide (50/50 molar ratio) having a viscosity midpoint of about 0.4 dl/g, about 0.5 wt % to about 5 wt % of polyethylene glycol 100 k, and about 0.005 wt % to about 0.2 wt % color-absorbing dye E172. In one example of a time-dependent disintegrating matrix, the time-dependent disintegrating matrix comprises about 45 wt % to about 55 wt % PCL (such as PCL having a viscosity midpoint between about 1.0 dl/g to about 1.4 dl/g), about 30 wt % to about 40 wt % of acid terminated copolymer of DL-lactide and glycolide (50/50 molar ratio) having a viscosity midpoint of about 0.4 dl/g, about 8 wt % to about 18 wt % of ester terminated copolymer of DL-lactide and glycolide (50/50 molar ratio) having a viscosity midpoint of about 0.4 dl/g, about 0.5 wt % to about 5 wt % of polyethylene glycol 100 k, and about 0.005 wt % to about 0.2 wt % color-absorbing dye E172. In one example of a time-dependent disintegrating matrix, the time-dependent disintegrating matrix comprises about 45 wt % to about 55 wt % PCL (such as PCL having a viscosity midpoint between about 1.0 dl/g to about 1.4 dl/g), about 27 wt % to about 37 wt % of acid terminated copolymer of DL-lactide and glycolide (50/50 molar ratio) having a viscosity midpoint of about 0.4 dl/g, about 12 wt % to about 22 wt % of ester terminated copolymer of DL-lactide and glycolide (50/50 molar ratio) having a viscosity midpoint of about 0.4 dl/g, about 0.5 wt % to about 5 wt % of polyethylene glycol 100 k, and about 0.005 wt % to about 0.2 wt % color-absorbing dye E172.

In another example of a time-dependent disintegrating matrix, the time-dependent disintegrating matrix comprises about 43 wt % to about 47 wt % PCL, about 33 wt % to about 37 wt % of acid terminated copolymer of DL-lactide and glycolide (50/50 molar ratio) having a viscosity midpoint of about 0.4 dl/g, about 15 wt % to about 20 wt % of copolymer of DL-lactide and glycolide (50/50 molar ratio) having a viscosity midpoint of about 0.4 dl/g, about 1 wt % to about 3 wt % of polyethylene glycol 100 k, and about 0.01 wt % to about 0.1 wt % color-absorbing dye E172. In one example of a time-dependent disintegrating matrix, the time-dependent disintegrating matrix comprises about 43 wt % to about 47 wt % PCL (such as PCL having a viscosity midpoint between about 1.5 dl/g to about 1.9 dl/g), about 33 wt % to about 37 wt % of acid terminated copolymer of DL-lactide and glycolide (50/50 molar ratio) having a viscosity midpoint of about 0.4 dl/g, about 15 wt % to about 20 wt % of ester terminated copolymer of DL-lactide and glycolide (50/50 molar ratio) having a viscosity midpoint of about 0.4 dl/g, about 1 wt % to about 3 wt % of polyethylene glycol 100 k, and about 0.01 wt % to about 0.1 wt % color-absorbing dye E172. In one example of a time-dependent disintegrating matrix, the time-dependent disintegrating matrix comprises about 43 wt % to about 47 wt % PCL (such as PCL having a viscosity midpoint between about 1.0 dl/g to about 1.4 dl/g), about 33 wt % to about 37 wt % of acid terminated copolymer of DL-lactide and glycolide (50/50 molar ratio) having a viscosity midpoint of about 0.4 dl/g, about 15 wt % to about 20 wt % of ester terminated copolymer of DL-lactide and glycolide (50/50 molar ratio) having a viscosity midpoint of about 0.4 dl/g, about 1 wt % to about 3 wt % of polyethylene glycol 100 k, and about 0.01 wt % to about 0.1 wt % color-absorbing dye E172. In one example of a time-dependent disintegrating matrix, the time-dependent disintegrating matrix comprises about 48 wt % to about 52 wt % PCL (such as PCL having a viscosity midpoint between about 1.0 dl/g to about 1.4 dl/g), about 30 wt % to about 34 wt % of acid terminated copolymer of DL-lactide and glycolide (50/50 molar ratio) having a viscosity midpoint of about 0.4 dl/g, about 14 wt % to about 18 wt % of ester terminated copolymer of DL-lactide and glycolide (50/50 molar ratio) having a viscosity midpoint of about 0.4 dl/g, about 1 wt % to about 3 wt % of polyethylene glycol 100 k, and about 0.01 wt % to about 0.1 wt % color-absorbing dye E172. In one example of a time-dependent disintegrating matrix, the time-dependent disintegrating matrix comprises about 48 wt % to about 52 wt % PCL (such as PCL having a viscosity midpoint between about 1.0 dl/g to about 1.4 d/g), about 36 wt % to about 40 wt % of acid terminated copolymer of DL-lactide and glycolide (50/50 molar ratio) having a viscosity midpoint of about 0.4 dl/g, about 8 wt % to about 12 wt % of ester terminated copolymer of DL-lactide and glycolide (50/50 molar ratio) having a viscosity midpoint of about 0.4 dl/g, about 1 wt % to about 3 wt % of polyethylene glycol 100 k, and about 0.01 wt % to about 0.1 wt % color-absorbing dye E172. In one example of a time-dependent disintegrating matrix, the time-dependent disintegrating matrix comprises about 48 wt % to about 52 wt % PCL (such as PCL having a viscosity midpoint between about 1.0 dl/g to about 1.4 dl/g), about 33 wt % to about 37 wt % of acid terminated copolymer of DL-lactide and glycolide (50/50 molar ratio) having a viscosity midpoint of about 0.4 dl/g, about 11 wt % to about 15 wt % of ester terminated copolymer of DL-lactide and glycolide (50/50 molar ratio) having a viscosity midpoint of about 0.4 dl/g, about 1 wt % to about 3 wt % of polyethylene glycol 100 k, and about 0.01 wt % to about 0.1 wt % color-absorbing dye E172. In one example of a time-dependent disintegrating matrix, the time-dependent disintegrating matrix comprises about 48 wt % to about 52 wt % PCL (such as PCL having a viscosity midpoint between about 1.0 dl/g to about 1.4 dl/g), about 30 wt % to about 34 wt % of acid terminated copolymer of DL-lactide and glycolide (50/50 molar ratio) having a viscosity midpoint of about 0.4 dl/g, about 14 wt % to about 18 wt % of ester terminated copolymer of DL-lactide and glycolide (50/50 molar ratio) having a viscosity midpoint of about 0.4 dl/g, about 1.5 wt % to about 3.5 wt % of polyethylene glycol 100 k, and about 0.01 wt % to about 0.1 wt % color-absorbing dye E172.

In another example of a time-dependent disintegrating matrix, the time-dependent disintegrating matrix comprises about 44.95 wt % PCL, about 35 wt % of acid terminated copolymer of DL-lactide and glycolide (50/50 molar ratio) having a viscosity midpoint of about 0.4 dl/g, about 18 wt % of copolymer of DL-lactide and glycolide (50/50 molar ratio) having a viscosity midpoint of about 0.4 dl/g, about 2 wt % of polyethylene glycol 100 k and about 0.05 wt % color-absorbing dye E172. In one example of a time-dependent disintegrating matrix, the time-dependent disintegrating matrix comprises about 44.95 wt % PCL (such as PCL having a viscosity midpoint of about 1.7 dl/g, such as Corbion PC 17), about 35 wt % of acid terminated copolymer of DL-lactide and glycolide (50/50 molar ratio) having a viscosity midpoint of about 0.4 dl/g, about 18 wt % of ester-terminated copolymer of DL-lactide and glycolide (50/50 molar ratio) having a viscosity midpoint of about 0.4 dl/g, about 2 wt % of polyethylene glycol 100 k and about 0.05 wt % color-absorbing dye E172. In one example of a time-dependent disintegrating matrix, the time-dependent disintegrating matrix comprises about 44.95 wt % PCL (such as PCL having a viscosity midpoint of about 1.2 dl/g, such as Corbion PC 12), about 35 wt % of acid terminated copolymer of DL-lactide and glycolide (50/50 molar ratio) having a viscosity midpoint of about 0.4 dl/g, about 18 wt % of ester-terminated copolymer of DL-lactide and glycolide (50/50 molar ratio) having a viscosity midpoint of about 0.4 dl/g, about 2 wt % of polyethylene glycol 100 k and about 0.05 wt % color-absorbing dye E172. In one example of a time-dependent disintegrating matrix, the time-dependent disintegrating matrix comprises about 49.95 wt % PCL (such as PCL having a viscosity midpoint of about 1.2 dl/g, such as Corbion PC 12), about 32 wt % of acid terminated copolymer of DL-lactide and glycolide (50/50 molar ratio) having a viscosity midpoint of about 0.4 dl/g, about 16 wt % of ester-terminated copolymer of DL-lactide and glycolide (50/50 molar ratio) having a viscosity midpoint of about 0.4 dl/g, about 2 wt % of polyethylene glycol 100 k and about 0.05 wt % color-absorbing dye E172. In one example of a time-dependent disintegrating matrix, the time-dependent disintegrating matrix comprises about 49.95 wt % PCL (such as PCL having a viscosity midpoint of about 1.2 dl/g, such as Corbion PC 12), about 38 wt % of acid terminated copolymer of DL-lactide and glycolide (50/50 molar ratio) having a viscosity midpoint of about 0.4 dl/g, about 10 wt % of ester-terminated copolymer of DL-lactide and glycolide (50/50 molar ratio) having a viscosity midpoint of about 0.4 dl/g, about 2 wt % of polyethylene glycol 100 k and about 0.05 wt % color-absorbing dye E172. In one example of a time-dependent disintegrating matrix, the time-dependent disintegrating matrix comprises about 49.95 wt % PCL (such as PCL having a viscosity midpoint of about 1.2 dl/g, such as Corbion PC 12), about 35 wt % of acid terminated copolymer of DL-lactide and glycolide (50/50 molar ratio) having a viscosity midpoint of about 0.4 dl/g, about 13 wt % of ester-terminated copolymer of DL-lactide and glycolide (50/50 molar ratio) having a viscosity midpoint of about 0.4 dl/g, about 2 wt % of polyethylene glycol 100 k and about 0.05 wt % color-absorbing dye E172. In one example of a time-dependent disintegrating matrix, the time-dependent disintegrating matrix comprises about 49.95 wt % PCL (such as PCL having a viscosity midpoint of about 1.2 dl/g, such as Corbion PC 12), about 31.75 wt % of acid terminated copolymer of DL-lactide and glycolide (50/50 molar ratio) having a viscosity midpoint of about 0.4 dl/g, about 15.75 wt % of ester-terminated copolymer of DL-lactide and glycolide (50/50 molar ratio) having a viscosity midpoint of about 0.4 dl/g, about 2.5 wt % of polyethylene glycol 100 k and about 0.05 wt % color-absorbing dye E172.

In some embodiments, a dosage form for administration of one or more agents comprises a gastric residence system, wherein the gastric residence system comprises a time-dependent disintegrating matrix comprising about 44.95 wt % of polycaprolactone (PCL), such as PCL having a viscosity midpoint between about 1.5 dl/g to about 2.1 dl/g, such as Corbion PC17. In some embodiments, the gastric residence system comprises a time-dependent disintegrating matrix comprising about 35.0 wt % of an acid terminated copolymer of DL-lactide and glycolide (50/50 molar ratio) having a viscosity midpoint between about 0.32 dl/g to about 0.48 dl/g (such as about 0.4 dl/g), such as PDLG 5004A. In some embodiments, the gastric residence system comprises a time-dependent disintegrating matrix comprising about 18.0 wt % of a copolymer of DL-lactide and glycolide (50/50 molar ratio) having a viscosity midpoint between about 0.32 dl/g to about 0.48 dl/g (such as about 0.4 dl/g), such as PDLG 5004. In some embodiments, the gastric residence system comprises a time-dependent disintegrating matrix comprising about 2.0 wt % of polyethylene glycol, such as polyethylene glycol with average molecular weight of 100,000, such as PEO_{100K}. In some embodiments, the gastric residence system comprises a time-dependent disintegrating matrix comprising about 0.05 wt % of iron oxide, such as E172. In some embodiments, a dosage form for administration of one or more agents comprises a gastric residence system, wherein the gastric residence system comprises a time-dependent disintegrating matrix comprising about 44.95 wt % of Corbion PC17, about 35.0 wt % of PDLG 5004A, about 18.0 wt % of PDLG 5004, about 2.0 wt % of PEO_{100K}, and about 0.05 wt % of E172.

In some embodiments, a dosage form for administration of one or more agents comprises a gastric residence system, wherein the gastric residence system comprises a time-dependent disintegrating matrix comprising about 44.95 wt % of polycaprolactone (PCL), such as PCL having a viscosity midpoint between about 1.0 dl/g to about 1.4 dl/g, such as 1.2 dl/g, such as Corbion PC12. In some embodiments, the gastric residence system comprises a time-dependent disintegrating matrix comprising about 35.0 wt % of an acid terminated copolymer of DL-lactide and glycolide (50/50 molar ratio) having a viscosity midpoint between about 0.32 dl/g to about 0.48 dl/g (such as about 0.4 dl/g), such as PDLG 5004A. In some embodiments, the gastric residence system comprises a time-dependent disintegrating matrix comprising about 18.0 wt % of an ester-terminated copolymer of DL-lactide and glycolide (50/50 molar ratio) having a viscosity midpoint between about 0.32 dl/g to about 0.48 dl/g (such as about 0.4 dl/g), such as PDLG 5004. In some embodiments, the gastric residence system comprises a time-dependent disintegrating matrix comprising about 2.0 wt % of polyethylene glycol, such as polyethylene glycol with average molecular weight of 100,000, such as PEO_{100K}. In some embodiments, the gastric residence system comprises a time-dependent disintegrating matrix comprising about 0.05 wt % of iron oxide, such as E172. In some embodiments, a dosage form for administration of one or more agents comprises a gastric residence system, wherein the gastric residence system comprises a time-dependent disintegrating matrix comprising about 44.95 wt % of Corbion PC12, about 35.0 wt % of PDLG 5004A, about 18.0 wt % of PDLG 5004, about 2.0 wt % of PEO_{100K}, and about 0.05 wt % of E172.

In some embodiments, a dosage form for administration of one or more agents comprises a gastric residence system, wherein the gastric residence system comprises a time-dependent disintegrating matrix comprising about 49.95 wt % of polycaprolactone (PCL), such as PCL having a viscosity midpoint between about 1.0 dl/g to about 1.4 dl/g, such as 1.2 dl/g, such as Corbion PC12. In some embodiments, the gastric residence system comprises a time-dependent disintegrating matrix comprising about 32.0 wt % of an acid terminated copolymer of DL-lactide and glycolide (50/50 molar ratio) having a viscosity midpoint between about 0.32 d/g to about 0.48 dl/g (such as about 0.4 dl/g), such as PDLG 5004A. In some embodiments, the gastric residence system comprises a time-dependent disintegrating matrix comprising about 16.0 wt % of an ester-terminated copolymer of DL-lactide and glycolide (50/50 molar ratio) having a viscosity midpoint between about 0.32 dl/g to about 0.48 dl/g (such as about 0.4 dl/g), such as PDLG 5004. In some embodiments, the gastric residence system comprises a time-dependent disintegrating matrix comprising about 2.0 wt % of polyethylene glycol, such as polyethylene glycol with average molecular weight of 100,000, such as PEO_{100K}. In some embodiments, the gastric residence system comprises a time-dependent disintegrating matrix comprising about 0.05 wt % of iron oxide, such as E172. In some embodiments, a dosage form for administration of one or more agents comprises a gastric residence system, wherein the gastric residence system comprises a time-dependent disintegrating matrix comprising about 49.95 wt % of Corbion PC12, about 32.0 wt % of PDLG 5004A, about 16.0 wt % of PDLG 5004, about 2.0 wt % of PEO_{100K}, and about 0.05 wt % of E172.

In some embodiments, a dosage form for administration of one or more agents comprises a gastric residence system, wherein the gastric residence system comprises a time-dependent disintegrating matrix comprising about 49.95 wt % of polycaprolactone (PCL), such as PCL having a viscosity midpoint between about 1.0 dl/g to about 1.4 dl/g, such as 1.2 dl/g, such as Corbion PC12. In some embodiments, the gastric residence system comprises a time-dependent disintegrating matrix comprising about 38.0 wt % of an acid terminated copolymer of DL-lactide and glycolide (50/50 molar ratio) having a viscosity midpoint between about 0.32 d/g to about 0.48 dl/g (such as about 0.4 dl/g), such as PDLG 5004A. In some embodiments, the gastric residence system comprises a time-dependent disintegrating matrix comprising about 10.0 wt % of an ester-terminated copolymer of DL-lactide and glycolide (50/50 molar ratio) having a viscosity midpoint between about 0.32 dl/g to about 0.48 dl/g (such as about 0.4 dl/g), such as PDLG 5004. In some embodiments, the gastric residence system comprises a time-dependent disintegrating matrix comprising about 2.0 wt % of polyethylene glycol, such as polyethylene glycol with average molecular weight of 100,000, such as PEO_{100K}. In some embodiments, the gastric residence system comprises a time-dependent disintegrating matrix comprising about 0.05 wt % of iron oxide, such as E172. In some embodiments, a dosage form for administration of one or more agents comprises a gastric residence system, wherein the gastric residence system comprises a time-dependent disintegrating matrix comprising about 49.95 wt % of Corbion PC12, about 38.0 wt % of PDLG 5004A, about 10.0 wt % of PDLG 5004, about 2.0 wt % of PEO_{100K}, and about 0.05 wt % of E172.

In some embodiments, a dosage form for administration of one or more agents comprises a gastric residence system, wherein the gastric residence system comprises a time-dependent disintegrating matrix comprising about 49.95 wt % of polycaprolactone (PCL), such as PCL having a viscosity midpoint between about 1.0 dl/g to about 1.4 dl/g, such as 1.2 dl/g, such as Corbion PC12. In some embodiments, the gastric residence system comprises a time-dependent disintegrating matrix comprising about 35.0 wt % of an acid terminated copolymer of DL-lactide and glycolide (50/50 molar ratio) having a viscosity midpoint between about 0.32 dl/g to about 0.48 dl/g (such as about 0.4 dl/g), such as PDLG 5004A. In some embodiments, the gastric residence system comprises a time-dependent disintegrating matrix comprising about 13.0 wt % of an ester-terminated copolymer of DL-lactide and glycolide (50/50 molar ratio) having a viscosity midpoint between about 0.32 dl/g to about 0.48 dl/g (such as about 0.4 dl/g), such as PDLG 5004. In some embodiments, the gastric residence system comprises a time-dependent disintegrating matrix comprising about 2.0 wt % of polyethylene glycol, such as polyethylene glycol with average molecular weight of 100,000, such as PEO_{100K}. In some embodiments, the gastric residence system comprises a time-dependent disintegrating matrix comprising about 0.05 wt % of iron oxide, such as E172. In some embodiments, a dosage form for administration of one or more agents comprises a gastric residence system, wherein the gastric residence system comprises a time-dependent disintegrating matrix comprising about 49.95 wt % of Corbion PC12, about 35.0 wt % of PDLG 5004A, about 13.0 wt % of PDLG 5004, about 2.0 wt % of PEO_{100K}, and about 0.05 wt % of E172.

In some embodiments, a dosage form for administration of one or more agents comprises a gastric residence system, wherein the gastric residence system comprises a time-dependent disintegrating matrix comprising about 49.95 wt % of polycaprolactone (PCL), such as PCL having a viscosity midpoint between about 1.0 dl/g to about 1.4 dl/g, such as 1.2 dl/g, such as Corbion PC12. In some embodiments, the gastric residence system comprises a time-dependent disintegrating matrix comprising about 31.75 wt % of an acid terminated copolymer of DL-lactide and glycolide (50/50 molar ratio) having a viscosity midpoint between about 0.32 d/g to about 0.48 dl/g (such as about 0.4 dl/g), such as PDLG 5004A. In some embodiments, the gastric residence system comprises a time-dependent disintegrating matrix comprising about 15.75 wt % of an ester-terminated copolymer of DL-lactide and glycolide (50/50 molar ratio) having a viscosity midpoint between about 0.32 dl/g to about 0.48 dl/g (such as about 0.4 dl/g), such as PDLG 5004. In some embodiments, the gastric residence system comprises a time-dependent disintegrating matrix comprising about 2.5 wt % of polyethylene glycol, such as polyethylene glycol with average molecular weight of 100,000, such as PEO_{100K}. In some embodiments, the gastric residence system comprises a time-dependent disintegrating matrix comprising about 0.05 wt % of iron oxide, such as E172. In some embodiments, a dosage form for administration of one or more agents comprises a gastric residence system, wherein the gastric residence system comprises a time-dependent disintegrating matrix comprising about 49.95 wt % of Corbion PC12, about 31.75 wt % of PDLG 5004A, about 15.75 wt % of PDLG 5004, about 2.5 wt % of PEO_{100K}, and about 0.05 wt % of E172.

Exemplary amounts of the components for a time-dependent disintegrating matrix are provided in the table below. The amounts are given in approximate weight percent, with the understanding that when ranges are provided, the amounts are chosen so as to add up to 100%.

| Time-dependent | | | |
|---|---|---|---|
| disintegrating matrix | Formulation 1 | Formulation 2 | Formulation 3 |
| PCL | 40-50 | 43-47 | 44.95 |
| PDLG5004A | 30-40 | 33-37 | 35 |
| PDLG5004 | 10-25 | 15-20 | 18 |
| PEO(100 k) | 0.5-5 | 1-3 | 2 |
| coloring (optional) | 0.005-0.2 | 0.01-0.1 | 0.05 (e.g. E172) |

Exemplary amounts of the components for a time-dependent disintegrating matrix are provided in the table below. The amounts are given in approximate weight percent, with the understanding that when ranges are provided, the amounts are chosen so as to add up to 100%.

| Time-dependent | | | |
|---|---|---|---|
| disintegrating | | | |
| matrix (T-DM1) | Formulation 1 | Formulation 2 | Formulation 3 |
| PCL (viscosity | 40-50 | 43-47 | 44.95 |
| midpoint 1.7 dl/g) | | | |
| PDLG5004A | 30-40 | 33-37 | 35 |
| PDLG5004 | 10-25 | 15-20 | 18 |
| PEO(100 k) | 0.5-5 | 1-3 | 2 |
| coloring (optional) | 0.005-0.2 | 0.01-0.1 | 0.05 (e.g. E172) |

Exemplary amounts of the components for a time-dependent disintegrating matrix are provided in the table below. The amounts are given in approximate weight percent, with the understanding that when ranges are provided, the amounts are chosen so as to add up to 100%.

| Time-dependent | | | |
|---|---|---|---|
| disintegrating | | | |
| matrix (T-DM2) | Formulation 1 | Formulation 2 | Formulation 3 |
| PCL (viscosity | 40-50 | 43-47 | 44.95 |
| midpoint 1.2 dl/g) | | | |
| PDLG5004A | 30-40 | 33-37 | 35 |
| PDLG5004 | 10-25 | 15-20 | 18 |
| PEO(100 k) | 0.5-5 | 1-3 | 2 |
| coloring (optional) | 0.005-0.2 | 0.01-0.1 | 0.05 (e.g. E172) |

Exemplary amounts of the components for a time-dependent disintegrating matrix are provided in the table below. The amounts are given in approximate weight percent, with the understanding that when ranges are provided, the amounts are chosen so as to add up to 100%.

| Time-dependent | | | |
|---|---|---|---|
| disintegrating | | | |
| matrix (T-DM3) | Formulation 1 | Formulation 2 | Formulation 3 |
| PCL (viscosity | 45-55 | 48-52 | 49.95 |
| midpoint 1.2 dl/g) | | | |
| PDLG5004A | 27-37 | 30-34 | 32 |
| PDLG5004 | 10-22 | 14-18 | 16 |
| PEO(100 k) | 0.5-5 | 1-3 | 2 |
| coloring (optional) | 0.005-0.2 | 0.01-0.1 | 0.05 (e.g. E172) |

Exemplary amounts of the components for a time-dependent disintegrating matrix are provided in the table below. The amounts are given in approximate weight percent, with the understanding that when ranges are provided, the amounts are chosen so as to add up to 100%.

| Time-dependent | | | |
|---|---|---|---|
| disintegrating | | | |
| matrix (T-DM4) | Formulation 1 | Formulation 2 | Formulation 3 |
| PCL (viscosity | 45-55 | 48-52 | 49.95 |
| midpoint 1.2 dl/g) | | | |
| PDLG5004A | 33-43 | 36-40 | 38 |
| PDLG5004 | 5-15 | 8-12 | 10 |
| PEO(100 k) | 0.5-5 | 1-3 | 2 |
| coloring (optional) | 0.005-0.2 | 0.01-0.1 | 0.05 (e.g. E172) |

Exemplary amounts of the components for a time-dependent disintegrating matrix are provided in the table below. The amounts are given in approximate weight percent, with the understanding that when ranges are provided, the amounts are chosen so as to add up to 100%.

| Time-dependent | | | |
|---|---|---|---|
| disintegrating | | | |
| matrix (T-DM5) | Formulation 1 | Formulation 2 | Formulation 3 |
| PCL (viscosity | 45-55 | 48-52 | 49.95 |
| midpoint 1.2 dl/g) | | | |
| PDLG5004A | 30-40 | 33-37 | 35 |
| PDLG5004 | 8-18 | 11-15 | 13 |
| PEO(100 k) | 0.5-5 | 1-3 | 2 |
| coloring (optional) | 0.005-0.2 | 0.01-0.1 | 0.05 (e.g. E172) |

Exemplary amounts of the components for a time-dependent disintegrating matrix are provided in the table below. The amounts are given in approximate weight percent, with the understanding that when ranges are provided, the amounts are chosen so as to add up to 100%.

| Time-dependent | | | |
|---|---|---|---|
| disintegrating | | | |
| matrix (T-DM6) | Formulation 1 | Formulation 2 | Formulation 3 |
| PCL (viscosity | 45-55 | 48-52 | 49.95 |
| midpoint 1.2 dl/g) | | | |
| PDLG5004A | 27-37 | 30-34 | 31.75 |
| PDLG5004 | 10-22 | 14-18 | 15.75 |
| PEO(100 k) | 0.5-5 | 1.5-3.5 | 2.5 |
| coloring (optional) | 0.005-0.2 | 0.01-0.1 | 0.05 (e.g. E172) |

### Gastric Residence Time

The gastric residence time of the system is controlled by the degradation or weakening, or breakage, rate of the time-dependent polymeric linker in the gastric residence system. Faster degradation or weakening, or breakage of the time-dependent polymeric linker results in faster passage of the system from the stomach. The residence time of the gastric residence system is defined as the time between administration of the system to the stomach and exit of the system from the stomach. In one embodiment, the gastric residence system has a residence time of about 24 hours, or up to about 24 hours. In one embodiment, the gastric residence system has a residence time of about 48 hours, or up to about 48 hours. In one embodiment, the gastric residence system has a residence time of about 72 hours, or up to about 72 hours. In one embodiment, the gastric residence system has a residence time of about 96 hours, or up to about 96 hours. In one embodiment, the gastric residence system has a residence time of about 5 days, or up to about 5 days. In one embodiment, the gastric residence system has a residence time of about 6 days, or up to about 6 days. In one embodiment, the gastric residence system has a residence time of about 7 days (about one week), or up to about 7 days (about one week). In one embodiment, the gastric residence system has a residence time of about 10 days, or up to about 10 days. In one embodiment, the gastric residence system has a residence time of about 14 days (about two weeks), or up to about 14 days (about two weeks).

In one embodiment, the gastric residence system has a residence time between about 24 hours and about 7 days. In one embodiment, the gastric residence system has a residence time between about 48 hours and about 7 days. In one embodiment, the gastric residence system has a residence time between about 72 hours and about 7 days. In one embodiment, the gastric residence system has a residence time between about 96 hours and about 7 days. In one embodiment, the gastric residence system has a residence time between about 5 days and about 7 days. In one embodiment, the gastric residence system has a residence time between about 6 days and about 7 days.

In one embodiment, the gastric residence system has a residence time between about 24 hours and about 10 days. In one embodiment, the gastric residence system has a residence time between about 48 hours and about 10 days. In one embodiment, the gastric residence system has a residence time between about 72 hours and about 10 days. In one embodiment, the gastric residence system has a residence time between about 96 hours and about 10 days. In one embodiment, the gastric residence system has a residence time between about 5 days and about 10 days. In one embodiment, the gastric residence system has a residence time between about 6 days and about 10 days. In one embodiment, the gastric residence system has a residence time between about 7 days and about 10 days.

In one embodiment, the gastric residence system has a residence time between about 24 hours and about 14 days. In one embodiment, the gastric residence system has a residence time between about 48 hours and about 14 days. In one embodiment, the gastric residence system has a residence time between about 72 hours and about 14 days. In one embodiment, the gastric residence system has a residence time between about 96 hours and about 14 days. In one embodiment, the gastric residence system has a residence time between about 5 days and about 14 days. In one embodiment, the gastric residence system has a residence time between about 6 days and about 14 days. In one embodiment, the gastric residence system has a residence time between about 7 days and about 14 days. In one embodiment, the gastric residence system has a residence time between about 10 days and about 14 days.

The gastric residence system releases a therapeutically effective amount of agent (or salt thereof) during at least a portion of the residence time or residence period during which the system resides in the stomach. In one embodiment, the system releases a therapeutically effective amount of agent (or salt thereof) during at least about 25% of the residence time. In one embodiment, the system releases a therapeutically effective amount of agent (or salt thereof) during at least about 50% of the residence time. In one embodiment, the system releases a therapeutically effective amount of agent (or salt thereof) during at least about 60% of the residence time. In one embodiment, the system releases a therapeutically effective amount of agent (or salt thereof) during at least about 70% of the residence time. In one embodiment, the system releases a therapeutically effective amount of agent (or salt thereof) during at least about 75% of the residence time. In one embodiment, the system releases a therapeutically effective amount of agent (or salt thereof) during at least about 80% of the residence time. In one embodiment, the system releases a therapeutically effective amount of agent (or salt thereof) during at least about 85% of the residence time. In one embodiment, the system releases a therapeutically effective amount of agent (or salt thereof) during at least about 90% of the residence time. In one embodiment, the system releases a therapeutically effective amount of agent (or salt thereof) during at least about 95% of the residence time. In one embodiment, the system releases a therapeutically effective amount of agent (or salt thereof) during at least about 98% of the residence time. In one embodiment, the system releases a therapeutically effective amount of agent (or salt thereof) during at least about 99% of the residence time. Enteric Disintegrating Matrices (Enteric Linkers)

The pH-dependent disintegrating matrices provide a safety mechanism for the gastric residence systems. If the system exits the stomach prematurely, that is, with all of the time-dependent disintegrating matrices intact, the pH-dependent disintegrating matrices will degrade, dissolve, disassociate, or mechanically weaken in the high pH environment of the small intestine, permitting the gastric residence system to pass readily through the small intestine. In addition, after passage of the gastric residence system once the time-dependent disintegrating matrices degrade, dissolve, disassociate, or mechanically weaken in the gastric environment, exposure of the pH-dependent disintegrating matrices to the high pH of the small intestine will provide further weakening and/or break-up of the system, for ready passage through the small intestine.

If the gastric residence system passes prematurely into the small intestine in an intact form, the system may be designed to break down much more rapidly to avoid intestinal obstruction. This is readily accomplished by using an enteric polymeric linker that includes an enteric polymer in addition to an additional linker polymer (such as a carrier polymer), which weakens or degrades within the intestinal environment. Enteric polymers are relatively resistant to the acidic pH levels encountered in the stomach, but dissolve rapidly at the higher pH levels found in the duodenum. Use of enteric polymeric linkers as safety elements protects against undesired passage of the intact gastric residence system into the small intestine. The use of enteric polymeric linker also provides a manner of removing the gastric residence system prior to its designed residence time; should the system need to be removed, the patient can drink a mildly alkaline solution, such as a sodium bicarbonate solution, or take an antacid preparation such as hydrated magnesium hydroxide (milk of magnesia) or calcium carbonate, which will raise the pH level in the stomach and cause rapid degradation of the enteric polymeric linker.

Weakening or degradation of the enteric polymeric linker may be measured in references to a loss of the flexural modulus or breakage of the polymeric linker under a given condition (e.g., enteric conditions or gastric conditions). The enteric linkers weaken, degrade, or break in the intestinal environment relatively quickly, while retain much of their flexural modulus in the gastric environment. Stomach conditions may be simulated using an aqueous solution, such as fasted-state simulated gastric fluid (FaSSGF), at a pH of 1.6 and at 37° C., and intestinal conditions may be simulated using an aqueous solution, such as fasted-state simulated intestinal fluid (FaSSIF), at a pH 6.5 at 37° C.

In some embodiments, the enteric disintegrating matrix comprises hydroxypropyl methylcellulose acetate succinate (HPMCAS). For example, in some embodiments, the enteric disintegrating matrix includes about 60 wt % to about 70 wt % HPMCAS. In some embodiments, the enteric disintegrating matrix includes about 62 wt % to about 66 wt % HPMCAS. In some embodiments, the enteric disintegrating matrix includes about 63.95 wt % HPMCAS.

The enteric polymer is combined with one or more additional polymers (such as one or more carrier polymers) in the enteric linker, preferably in a homogenous mixture. For example, the enteric polymer and the additional linker polymer may be homogenously blended together before the mixture is extruded, and the extruded material being cut to a desired size for the polymeric linker. In some embodiments, the one or more additional linker polymers are miscible with the enteric polymer. The one or more additional linker polymers may be a non-degradable polymer (that is, not degradable or in the gastric or enteric environment, or an aqueous solution of pH 1.6 (representing the gastric environment) or pH 6.5 (representing the enteric environment).

Bonding of the polymeric linker to a directly adjacent member may be improved if at least one polymer is common to both the adjacent member and the enteric polymeric linker. That is, one of the one or more additional linker polymers in the enteric linker may be the same (or the same polymer type) as at least one polymer in a directly adjacent component (or, optionally, both directly adjacent components) of the gastric residence system. For example, if the enteric polymeric linker is bonded directly to a structural member comprising a carrier polymer, in some embodiments the one or more additional linker polymers also includes the carrier polymer (in addition to the PLGA in the time-dependent polymeric linker) at the same or different concentration. Exemplary carrier polymers include, but are not limited to, polylactic acid (PLA), polycaprolactone (PCL), and a thermoplastic polyurethane (TPU), among others described herein.

In some embodiments, the one or more additional linker polymers in the enteric linker comprises a PCL. The enteric polymeric linker may be directly joined or bonded to another member of the gastric residence system (such as the structural member comprising the drug and the carrier polymer, a coupling member, the time-dependent polymeric linker, or a central structural member), which may also include a PCL, which may be the same PCL in the enteric polymeric linker or a different PCL as the one in the enteric polymeric linker, and which may be at the same concentration or a different concentration. A different PCL in the enteric polymeric linker and the other member directly joined or bonded to the enteric linker may differ, for example, in the weight-average molecular weight of the PCL, the inherent viscosity of the PCL, or the proportions of PCL (for example, when a blend of two or more PCL polymers are used). In some embodiments, the enteric disintegrating matrix comprises about 30 wt % to about 40 wt % PCL. In some embodiments, the enteric disintegrating matrix comprises about 32 wt % to about 37 wt % PCL. In some embodiments, the enteric disintegrating matrix comprises about 34 wt % PCL. In some embodiments, the enteric disintegrating matrix comprises about 33.95 wt % PCL.

The enteric disintegrating matrix may further include one or more plasticizers, such as a poloxamer (e.g., Poloxamer 407, or “P407”). In some embodiments, the enteric disintegrating matrix comprises about 0.5 wt % to about 5 wt % poloxamer. In some embodiments, the enteric disintegrating matrix comprises about 1 wt % to about 3 wt % poloxamer. In some embodiments, the enteric disintegrating matrix comprises about 2 wt % poloxamer.

In some embodiments, the enteric disintegrating matrix includes a color-absorbing dyes (also referred to as a colorant or a pigment). A color-absorbing dye may be included to enhance bonding or attachment of the polymeric linker to other gastric residence system components. Color-absorbing dyes can absorb heat during the laser-welding, infrared welding, or other heat-induced attachment, which increases the tensile strength of the resulting bond. Exemplary color-absorbing dyes include iron oxide and carbon black. The enteric polymeric linker may include the color-absorbing dye in an amount of up to about 5%, such as up to about 4%, up to about 3%, up to about 2%, up to about 1%, up to about 0.5%, up to about 0.3%, up to about 0.2%, or up to about 0.1%. In some embodiments, the enteric disintegrating matrix comprises about 0.01 wt % to about 0.2 wt % color-absorbing dye E172. In some embodiments, the enteric disintegrating matrix comprises about 0.05 wt % to about 0.15 wt % color-absorbing dye E172. In some embodiments, the enteric disintegrating matrix comprises about 0.1 wt % color-absorbing dye E172.

In some embodiments, the enteric disintegrating matrix comprises about 59 wt % to about 69 wt % HPMCAS, about 29 wt % to about 39 wt % PCL, and about 0.5 wt % to about 5 wt % poloxamer (such as P407). Optionally, the enteric disintegrating matrix further comprises iron oxide, for example about 0.01 wt % to about 0.2 wt % iron oxide (such as E172).

In some embodiments, the enteric disintegrating matrix comprises about 62 wt % to about 66 wt % HPMCAS, about 32 wt % to about 36 wt % PCL, and about 1 wt % to about 3 wt % poloxamer (such as P407). Optionally, the enteric disintegrating matrix further comprises iron oxide, for example about 0.05 wt % to about 0.15 wt % iron oxide (such as E172).

In some embodiments, the enteric disintegrating matrix comprises about 63.95 wt % HPMCAS, about 33.95 wt % PCL, and about 2 wt % poloxamer (such as P407). Optionally, the enteric disintegrating matrix further comprises iron oxide, for example about 0.1 wt % iron oxide (such as E172).

In some embodiments, the enteric disintegrating matrix comprises about 59 wt % to about 69 wt % HPMCAS, about 29 wt % to about 39 wt % PCL, and about 0.5 wt % to about 5 wt % poloxamer (such as P407).

In some embodiments, the enteric disintegrating matrix comprises about 62 wt % to about 66 wt % HPMCAS, about 32 wt % to about 36 wt % PCL, and about 1 wt % to about 3 wt % poloxamer (such as P407).

In some embodiments, the enteric disintegrating matrix comprises about 64 wt % HPMCAS, about 34 wt % PCL, and about 2 wt % poloxamer (such as P407).

In some embodiments, a dosage form for administration of one or more agents comprises a gastric residence system, wherein the gastric residence system comprises a pH-dependent disintegrating matrix comprising about 33.95 wt % of polycaprolactone (PCL), such as PCL having a viscosity midpoint between about 1.5 dl/g to about 2.1 dl/g, such as Corbion PC17. In some embodiments, the gastric residence system comprises a pH-dependent disintegrating matrix comprising about 63.95 wt % of hypromellose acetate succinate, such as HPMCAS-MG. In some embodiments, the gastric residence system comprises a pH-dependent disintegrating matrix comprising about 2.0 wt % of poly(ethylene glycol)-block-poly(propylene glycol)-block-poly(ethylene glycol) polymers, such as H—(OCH2CH2)x-(O—CH(CH3)CH2)y-(OCH2CH2)z-OH where x and z are about 101 and y is about 56, such as Poloxamer 407 (P407, a poly(ethylene glycol)-block-poly(propylene glycol)-block-poly(ethylene glycol) polymer with a polyoxypropylene molecular mass of about 4000 and about 70% polyoxyethylene content). In some embodiments, the gastric residence system comprises a pH-dependent disintegrating matrix comprising about 0.1 wt % of iron oxide, such as E172. In some embodiments, a dosage form for administration of one or more agents comprises a gastric residence system, wherein the gastric residence system comprises a pH-dependent disintegrating matrix comprising about 33.95 wt % of Corbion PC17, about 63.95 wt % of HPMCAS-MG, about 2.0 wt % of P407, and about 0.1 wt % of E172.

Exemplary amounts of the components for the enteric disintegrating matrix are provided in the table below. The amounts are given in approximate weight percent, with the understanding that when ranges are provided, the amounts are chosen so as to add up to 100%.

| Enteric | | | |
|---|---|---|---|
| disintegrating | | | |
| matrix (E-DM1) | Formulation 1 | Formulation 2 | Formulation 3 |
| PCL (such as PC17 | 29-39 | 32-36 | 33.95 |
| with viscosity | | | |
| midpoint 1.7 dl/g) | | | |
| HPMCAS | 59-69 | 62-66 | 63.95 |
| P407 | 0.5-5 | 1-3 | 2 |
| coloring (optional) | 0.01-0.2 | 0.05-0.15 | 0.1 (e.g. E172) |

In some embodiments, a dosage form for administration of one or more agents comprises a gastric residence system, wherein the gastric residence system comprises a pH-dependent disintegrating matrix comprising about 34 wt % of polycaprolactone (PCL), such as PCL having a viscosity midpoint between about 1.5 dl/g to about 2.1 dl/g, such as Corbion PC17. In some embodiments, the gastric residence system comprises a pH-dependent disintegrating matrix comprising about 64 wt % of hypromellose acetate succinate, such as HPMCAS-MG. In some embodiments, the gastric residence system comprises a pH-dependent disintegrating matrix comprising about 2.0 wt % of poly(ethylene glycol)-block-poly(propylene glycol)-block-poly(ethylene glycol) polymers, such as H—(OCH2CH2)x-(O—CH(CH3)CH2)y-(OCH2CH2)z-OH where x and z are about 101 and y is about 56, such as Poloxamer 407 (P407, a poly(ethylene glycol)-block-poly(propylene glycol)-block-poly(ethylene glycol) polymer with a polyoxypropylene molecular mass of about 4000 and about 70% polyoxyethylene content). In some embodiments, a dosage form for administration of one or more agents comprises a gastric residence system, wherein the gastric residence system comprises a pH-dependent disintegrating matrix comprising about 34 wt % of Corbion PC17, about 64 wt % of HPMCAS-MG, and about 2.0 wt % of P407.

Exemplary amounts of the components for the enteric disintegrating matrix are provided in the table below. The amounts are given in approximate weight percent, with the understanding that when ranges are provided, the amounts are chosen so as to add up to 100%.

| Enteric | | | |
|---|---|---|---|
| disintegrating | | | |
| matrix (E-DM2) | Formulation 1 | Formulation 2 | Formulation 3 |
| PCL (such as PC17 | 29-39 | 32-36 | 34 |
| with viscosity | | | |
| midpoint 1.7 dl/g) | | | |
| HPMCAS | 59-69 | 62-66 | 64 |
| P407 | 0.5-5 | 1-3 | 2 |

### Disintegrating Filament

In some embodiments, the gastric residence system comprises arms that are linked by one or more filaments. In some embodiments, the filament is a disintegrating filament. In some embodiments, the gastric residence system comprises arms that are linked at the distal tip by one or more filaments. In some embodiments, the filament circumferentially connects the arms. In some embodiments, the filament is a disintegrating filament. In some embodiments, the filament comprises one or more of: poly (lactic-co-glycolic acid), polyglycolic acid, Polylactic acid, polydioxanone, polycaprolactone, polytrimethylene carbonate, cellulose, or any blends and copolymers thereof. In some embodiments, the filament comprises poly (lactic-co-glycolic acid). In some embodiments, the filament comprises polyglycolic acid. In some embodiments, the thickness of the filament is about any one of 0.05 mm, 0.1 mm, 0.15 mm, 0.20 mm, 0.25 mm, 0.30 mm, 0.35 mm, 0.40 mm, 0.45 mm, 0.5 mm, 0.6 mm, 0.7 mm, 0.8 mm, 0.9 mm, 1.0 mm or any thickness therebetween. In some embodiments, the thickness of the filament is about 0.20 mm. In some embodiments, the thickness of the filament is about 0.30 mm. In some embodiments, the filament is Bondek Suture 2-0. In some embodiments, the filament is Bondek Suture 3-0.

### Further Disintegrating Matrices as Arm Tip

In some embodiments, the gastric residence system comprises arms comprising a third disintegrating matrix in addition to the time-dependent disintegrating matrix and the enteric disintegrating matrix. In some embodiments, the third disintegrating matrix is a filament holding segment (i.e. segment to which the filament is attached). In some embodiments, the third disintegrating matrix is the distal segment of the residence system arm, i.e. the tip of the arm. In some embodiments, the third disintegrating matrix is referred to as outer disintegrating matrix tip enteric PCL (ODMTEP).

In some embodiments, the third disintegrating matrix comprises hydroxypropyl methylcellulose acetate succinate (HPMCAS). For example, in some embodiments the third disintegrating matrix includes about 60 wt % to about 70 wt % HPMCAS. In some embodiments, the third disintegrating matrix includes about 63 wt % to about 67 wt % HPMCAS. In some embodiments, the third disintegrating matrix includes about 64.9 wt % HPMCAS.

In some embodiments, the third disintegrating matrix comprises a polymer common with one or other segment in the gastric residence system arm. In some embodiments, the third disintegrating matrix comprises polycaprolactone (PCL). In some embodiments, the third disintegrating matrix comprises about 25 wt % to about 35 wt % PCL. In some embodiments, the third disintegrating matrix comprises about 28 wt % to about 32 wt % PCL. In some embodiments, the third disintegrating matrix comprises about 30 wt % PCL.

In some embodiments, the third disintegrating matrix comprises one or more acids, such as stearic acid. In some embodiments, the third disintegrating matrix comprises about 1 wt % to about 5 wt % stearic acid. In some embodiments, the third disintegrating matrix comprises about 2 wt % to about 3 wt % stearic acid. In some embodiments, the third disintegrating matrix comprises about 2.5 wt % stearic acid.

In some embodiments, the third disintegrating matrix may further include one or more plasticizers, such as a propylene glycol. In some embodiments, the third disintegrating matrix comprises about 1 wt % to about 5 wt % propylene glycol. In some embodiments, the third disintegrating matrix comprises about 2 wt % to about 3 wt % propylene glycol. In some embodiments, the third disintegrating matrix comprises about 2.5 wt % propylene glycol.

In some embodiments, the third disintegrating matrix includes a color-absorbing dyes (also referred to as a colorant or a pigment). A color-absorbing dye may be included to enhance bonding or attachment of the polymeric linker to other gastric residence system components. Color-absorbing dyes can absorb heat during the laser-welding, infrared welding, or other heat-induced attachment, which increases the tensile strength of the resulting bond. Exemplary color-absorbing dyes include iron oxide and carbon black. The third disintegrating matrix may include the color-absorbing dye in an amount of up to about 5%, such as up to about 4%, up to about 3%, up to about 2%, up to about 1%, up to about 0.5%, up to about 0.3%, up to about 0.2%, or up to about 0.1%. In some embodiments, the third disintegrating matrix comprises about 0.01 wt % to about 0.5 wt % color-absorbing dye. In some embodiments, the third disintegrating matrix comprises about 0.05 wt % to about 0.15 wt % color-absorbing dye. In some embodiments, the third disintegrating matrix comprises about 0.1 wt % color-absorbing dye. In some embodiments, the third disintegrating matrix comprises about 0.025% ferrosoferric oxide and about 0.075% FD&C Red 40. In some embodiments, the third disintegrating matrix comprises about 0.025% ferrosoferric oxide and about 0.075% FD&C Red 40.

In some embodiments, the third disintegrating matrix comprises about 60 wt % to about 70 wt % HPMCAS, about 25 wt % to about 35 wt % PCL, about 1 wt % to about 5 wt % propylene glycol and about 1 wt % to about 5 wt % stearic acid. Optionally, the third disintegrating matrix further comprises about 0.01 wt % to about 0.5 wt % iron oxide.

In some embodiments, the third disintegrating matrix comprises about 63 wt % to about 67 wt % HPMCAS, about 28 wt % to about 32 wt % PCL, about 2 wt % to about 3 wt % propylene glycol and about 2 wt % to about 3 wt % stearic acid. Optionally, the third disintegrating matrix further comprises about 0.05 wt % to about 0.15 wt % iron oxide.

In some embodiments, the third disintegrating matrix comprises 64.9 wt % HPMCAS, about 30 wt % PCL, about 2.5 wt % propylene glycol and about 2.5 wt % stearic acid. Optionally, the third disintegrating matrix further comprises about 0.1 wt % iron oxide, for example about 0.025% ferrosoferric oxide and about 0.075% FD&C Red 40.

Exemplary amounts of the components for the third disintegrating matrix are provided in the table below. The amounts are given in approximate weight percent, with the understanding that when ranges are provided, the amounts are chosen so as to add up to 100%.

| ODMTEP | | | |
|---|---|---|---|
| disintegrating | | | |
| matrix | Formulation 1 | Formulation 2 | Formulation 3 |
| PCL (such as PC17 | 25-35 | 28-32 | 30 |
| with viscosity | | | |
| midpoint 1.7 dl/g) | | | |
| HPMCAS | 60-70 | 63-67 | 64.9 |
| Stearic acid | 1-5 | 2-3 | 2.5 |
| Propylene Glycol | 1-5 | 2-3 | 2.5 |
| Coloring (e.g. | 0.01-0.5 | 0.05-0.15 | 0.1 (e.g. 0.025% |
| Iron oxide) | | | ferrosoferric oxide |
| | | | and 0.075% FD&C |
| | | | Red 40) |

### Inert Segments

In some embodiments, the gastric residence system comprises one or more inert segments. In some embodiments, the inert segment comprises one or more radiopaque substances.

In some embodiments, the inert segment comprises a common polymer with other segments in the gastric residence system. In some embodiments, the inert segment comprises polycaprolactone (PCL). In some embodiments, the inert segment comprises about 61 wt % to about 71 wt % PCL. In some embodiments, the inert segment comprises about 64 wt % to about 69 wt % PCL. In some embodiments, the inert segment comprises about 66.5 wt % PCL. In some embodiments, the inert segment comprises about 66.45 wt % PCL

In some embodiments, the inert segment comprises vinylpyrrolidone-vinyl acetate copolymer in a ratio of 6:4 by mass (i.e. copovidone, such as Kollidon VA64). In some embodiments, the inert segment comprises about 27 wt % to about 37 wt % copovidone. In some embodiments, the inert segment comprises about 30 wt % to about 34 wt % copovidone. In some embodiments, the inert segment comprises about 32 wt % copovidone.

The inert segment may further include one or more plasticizers, such as a poloxamer (e.g., Poloxamer 407, or “P407”). In some embodiments, the inert segment comprises about 0.2 wt % to about 4 wt % poloxamer. In some embodiments, the inert segment comprises about 0.5 wt % to about 2.5 wt % poloxamer. In some embodiments, the inert segment comprises about 1.5 wt % poloxamer.

In some embodiments, the inert segment includes a color-absorbing dyes (also referred to as a colorant or a pigment). The inert segment may include the color-absorbing dye in an amount of up to about 5%, such as up to about 4%, up to about 3%, up to about 2%, up to about 1%, up to about 0.5%, up to about 0.3%, up to about 0.2%, up to about 0.1%, or up to 0.05%. In some embodiments, the inert segment comprises about 0.005 wt % to about 0.2 wt % color-absorbing dye. In some embodiments, the inert segment comprises about 0.01 wt % to about 0.1 wt % color-absorbing dye. In some embodiments, the inert segment comprises about 0.05 wt % color-absorbing dye. In some embodiments, the color-absorbing dye is FD&C Blue #1.

In some embodiments, the inert segment comprises about 61 wt % to about 71 wt % PCL, about 27 wt % to about 37 wt % copovidone, about 0.2 wt % to about 4 wt % poloxamer. Optionally, the inert segment further comprises color-absorbing dye, for example about 0.005 wt % to about 0.2 wt % color-absorbing dye FD&C Blue #1.

In some embodiments, the inert segment comprises about 64 wt % to about 69 wt % PCL, about 30 wt % to about 34 wt % copovidone, about 0.5 wt % to about 2.5 wt % poloxamer. Optionally, the inert segment further comprises color-absorbing dye, for example about 0.01 wt % to about 0.1 wt % color-absorbing dye FD&C Blue #1.

In some embodiments, the inert segment comprises about 66.45 wt % PCL, about 32 wt % copovidone, about 1.5 wt % poloxamer. Optionally, the inert segment further comprises color-absorbing dye, for example about 0.05 wt % color-absorbing dye FD&C Blue #1.

Exemplary amounts of the components for one embodiment of the inert segment (e.g. inactive spacer) are provided in the table below. The amounts are given in approximate weight percent, with the understanding that when ranges are provided, the amounts are chosen so as to add up to 100%.

| Inert Segment | | | |
|---|---|---|---|
| (Inactive spacer) IS-1 | Formulation 1 | Formulation 2 | Formulation 3 |
| PCL (such as PC17 | 61-71 | 64-69 | 66.45 |
| with viscosity | | | |
| midpoint 1.7 dl/g) | | | |
| VA64 | 27-37 | 30-34 | 32 |
| P407 | 0.2-4 | 0.5-2.5 | 1.5 |
| coloring (optional) | 0.005-0.2 | 0.01-0.1 | 0.05 (e.g. |
| | | | Blue #1) |

In some embodiments, the inert segment comprises a common polymer with other segments in the gastric residence system. In some embodiments, the inert segment comprises polycaprolactone (PCL). In some embodiments, the inert segment comprises about 35 wt % to about 45 wt % PCL. In some embodiments, the inert segment comprises about 38 wt % to about 42 wt % PCL. In some embodiments, the inert segment comprises about 40 wt % PCL. In some embodiments, the inert segment comprises about 33.995 wt % PCL.

In some embodiments, the inert segment comprises vinylpyrrolidone-vinyl acetate copolymer in a ratio of 6:4 by mass (i.e. copovidone, such as Kollidon VA64). In some embodiments, the inert segment comprises about 37 wt % to about 47 wt % copovidone. In some embodiments, the inert segment comprises about 40 wt % to about 44 wt % copovidone. In some embodiments, the inert segment comprises about 42 wt % copovidone.

The inert segment may further include one or more plasticizers, such as a poloxamer (e.g., Poloxamer 407, or “P407”). In some embodiments, the inert segment comprises about 1 wt % to about 5 wt % poloxamer. In some embodiments, the inert segment comprises about 2 wt % to about 4 wt % poloxamer. In some embodiments, the inert segment comprises about 3 wt % poloxamer.

The inert segment may include one or more plasticizers, such as polyethylene glycol. The term “polyethylene glycol” is used interchangeably herein with the terms “polyethylene oxide” and “PEO.” In some embodiments, the molecular weight of the polyethylene glycol is about 90K to about 110K, such as 100 k (also referred to as 100K or 100 kDa). In some embodiments, the inert segment comprises polyethylene glycol with molecular weight of about 100 k (polyethylene glycol 100 k). In some embodiments, the inert segment comprises about 10 wt % to about 20 wt % polyethylene glycol 100 k. In some embodiments, the inert segment comprises about 13 wt % to about 17 wt % polyethylene glycol 100 k. In some embodiments, the inert segment comprises about 15 wt % polyethylene glycol 100 k.

In some embodiments, the inert segment includes a color-absorbing dyes (also referred to as a colorant or a pigment). In some embodiments, the inert segment may include the color-absorbing dye in an amount of up to about 1%, such as up to about 0.5%, up to about 0.4%, up to about 0.3%, up to about 2%, up to about 1%, up to about 0.5%, up to about 0.3%, up to about 0.2%, up to about 0.1%, or up to 0.005%. In some embodiments, the inert segment comprises about 0.0005 wt % to about 0.2 wt % color-absorbing dye. In some embodiments, the inert segment comprises about 0.001 wt % to about 0.01 wt % color-absorbing dye. In some embodiments, the inert segment comprises about 0.005 wt % color-absorbing dye. In some embodiments, the color-absorbing dye is iron oxide (such as E172).

In some embodiments, the inert segment comprises about 35 wt % to about 45 wt % PCL, about 37 wt % to about 47 wt % copovidone, about 10 wt % to about 20 wt % of polyethylene glycol, such as polyethylene glycol with average molecular weight of 100,000, such as PEO_{100K}, about 1 wt % to about 5 wt % poloxamer. Optionally, the inert segment further comprises color-absorbing dye, for example about 0.0005 wt % to about 0.02 wt % color-absorbing dye E172.

In some embodiments, the inert segment comprises about 38 wt % to about 42 wt % PCL, about 40 wt % to about 44 wt % copovidone, about 13 wt % to about 17 wt % of polyethylene glycol, such as polyethylene glycol with average molecular weight of 100,000, such as PEO100K, about 2 wt % to about 4 wt % poloxamer. Optionally, the inert segment further comprises color-absorbing dye, for example about 0.001 wt % to about 0.01 wt % color-absorbing dye E172.

In some embodiments, the inert segment comprises about 39.995 wt % PCL, about 42 wt % copovidone, about 15 wt % of PEO100K, and about 3 wt % poloxamer. Optionally, the inert segment further comprises color-absorbing dye, for example about 0.005 wt % color-absorbing dye E172.

Exemplary amounts of the components for one embodiment of the inert segment (e.g. inactive spacer) are provided in the table below. The amounts are given in approximate weight percent, with the understanding that when ranges are provided, the amounts are chosen so as to add up to 100%.

| Inert Segment | | | |
|---|---|---|---|
| (Inactive | | | |
| spacer) IS-2 | Formulation 1 | Formulation 2 | Formulation 3 |
| PCL (such as PC17 | 35-45 | 38-42 | 39.995 |
| with viscosity | | | |
| midpoint 1.7 dl/g) | | | |
| VA64 | 37-47 | 40-44 | 42 |
| PEO100k | 10-20 | 13-17 | 15 |
| P407 | 1-5 | 2-4 | 3 |
| coloring (optional) | 0.0005-0.02 | 0.001-0.01 | 0.005 (e.g. |
| | | | Blue #1) |

In some embodiments, the gastric residence system comprises one or more inert segments, wherein the inert segment comprises one or more radiopaque substances. In some embodiments, the gastric residence system comprises an inert segment, wherein the inert segment is a radiopaque segment.

In some embodiments, the inert segment comprises a common polymer with other segments in the gastric residence system. In some embodiments, the inert segment comprises polycaprolactone (PCL). In some embodiments, the inert segment comprises about 65 wt % to about 75 wt % PCL. In some embodiments, the inert segment comprises about 68 wt % to about 72 wt % PCL. In some embodiments, the inert segment comprises about 70 wt % PCL.

In some embodiments, the inert segment comprises a radiopaque substance. In some embodiments, the inert segment comprises a radiopaque substance, wherein the radiopaque substance is (BiO)₂CO₃. In some embodiments, the inert segment comprises (BiO)₂CO₃. In some embodiments, the inert segment comprises about 25 wt % to about 35 wt % (BiO)₂CO₃. In some embodiments, the inert segment comprises about 28 wt % to about 32 wt % (BiO)₂CO₃. In some embodiments, the inert segment comprises about 30 wt % (BiO)₂CO₃.

In some embodiments, the inert segment comprises about 65 wt % to about 75 wt % PCL, and about 25 wt % to about 35 wt % (BiO)₂CO₃. In some embodiments, the inert segment comprises about 68 wt % to about 72 wt % PCL, and about 28 wt % to about 32 wt % (BiO)₂CO₃. In some embodiments, the inert segment comprises about 70 wt % PCL, and about 30 wt % (BiO)₂CO₃.

Exemplary amounts of the components for one embodiment of the inert segment (e.g. rPCL segment) are provided in the table below. The amounts are given in approximate weight percent, with the understanding that when ranges are provided, the amounts are chosen so as to add up to 100%.

| Inert segment- | | | |
|---|---|---|---|
| rPCL (radiopaque) | | | |
| IS-3 | Formulation 1 | Formulation 2 | Formulation 3 |
| PCL (such as PC17 | 65-75 | 68-72 | 70 |
| with viscosity | | | |
| midpoint 1.7 dl/g) | | | |
| (BiO)₂CO₃ | 25-35 | 28-32 | 30 |

### Carrier Polymer-Agent Segments (Drug-Eluting Segments)

The carrier polymer-agent segments, or drug-eluting segments, release an agent in a controlled manner during the period that the gastric residence system resides in the stomach. The carrier polymer is blended with the agent, and formed into segments which are then assembled with the other components described herein to manufacture the gastric residence system. The composition of such carrier polymer-agent blends is provided below for specific drug formulations, including risperidone.

In some embodiments, a dosage form for administration of risperidone comprises a gastric residence system comprising about 10 mg to about 35 mg of risperidone. In some embodiments, a dosage form for administration of risperidone comprises a gastric residence system comprising about 10 mg to about 20 mg of risperidone. In some embodiments, a dosage form for administration of risperidone comprises a gastric residence system comprising about 14 mg of risperidone. In some embodiments, a dosage form for administration of risperidone comprises a gastric residence system comprising about 20 mg to about 35 mg of risperidone. In some embodiments, the dosage form comprises a gastric residence system, wherein the gastric residence system comprises about 28 mg of risperidone.

In some embodiments, the dosage form comprises a gastric residence system, wherein the gastric residence system comprises a drug-eluting segment comprising about 14 mg of risperidone. In some embodiments, the dosage form comprises a gastric residence system, wherein the gastric residence system comprises a drug-eluting segment comprising about 28 mg of risperidone. In some embodiments, wherein the drug-eluting segment comprises about 30 wt % to about 40 wt % of risperidone, the drug-eluting segment comprises about 51 wt % to about 61 wt % of polycaprolactone (PCL), such as PCL having a viscosity midpoint between about 1.5 dl/g to about 2.1 dl/g, such as Corbion PC17. In some embodiments, the drug-eluting segment comprises about 2 wt % to about 8 wt % of vinylpyrrolidone-vinyl acetate copolymer, such as Kollidon VA64. In some embodiments, the drug-eluting segment comprises about 1 wt % to about 5 wt % of poly(ethylene glycol)-block-poly(propylene glycol)-block-poly(ethylene glycol) polymers, such as H—(OCH2CH2)x-(O—CH(CH3)CH2)y-(OCH2CH2)z-OH where x and z are about 101 and y is about 56, such as Poloxamer 407. In some embodiments, the drug-eluting segment comprises about 0.1 wt % to about 1 wt % of Vitamin E succinate. In some embodiments, the drug-eluting segment comprises about 0.1 wt % to about 1 wt % of colloidal silicon dioxide (SiO₂). In some embodiments, the drug-eluting segment comprises about 0.01 wt % to about 0.5 wt % of pigment.

In some embodiments, wherein the drug-eluting segment comprises about 33 wt % to about 37 wt % of risperidone, the drug-eluting segment comprises about 54 wt % to about 58 wt % of polycaprolactone (PCL), such as PCL having a viscosity midpoint between about 1.5 dl/g to about 2.1 dl/g, such as Corbion PC17. In some embodiments, the drug-eluting segment comprises about 4 wt % to about 6 wt % of vinylpyrrolidone-vinyl acetate copolymer, such as Kollidon VA64. In some embodiments, the drug-eluting segment comprises about 2 wt % to about 4 wt % of poly(ethylene glycol)-block-poly(propylene glycol)-block-poly(ethylene glycol) polymers, such as H—(OCH2CH2)x-(O—CH(CH3)CH2)y-(OCH2CH2)z-OH where x and z are about 101 and y is about 56, such as Poloxamer 407. In some embodiments, the drug-eluting segment comprises about 0.2 wt % to about 0.8 wt % of Vitamin E succinate. In some embodiments, the drug-eluting segment comprises about 0.2 wt % to about 0.8 wt % of colloidal silicon dioxide (SiO₂). In some embodiments, the drug-eluting segment comprises about 0.05 wt % to about 0.2 wt % of pigment.

In some embodiments, wherein the drug-eluting segment comprises about 35 wt % of risperidone, the drug-eluting segment comprises about 55.9 wt % of polycaprolactone (PCL), such as PCL having a viscosity midpoint between about 1.5 dl/g to about 2.1 dl/g, such as Corbion PC17. In some embodiments, the drug-eluting segment comprises about 5.0 wt % of vinylpyrrolidone-vinyl acetate copolymer, such as Kollidon VA64. In some embodiments, the drug-eluting segment comprises about 3.0 wt % of poly(ethylene glycol)-block-poly(propylene glycol)-block-poly(ethylene glycol) polymers, such as H—(OCH2CH2)x-(O—CH(CH3)CH2)y-(OCH2CH2)z-OH where x and z are about 101 and y is about 56, such as Poloxamer 407. In some embodiments, the drug-eluting segment comprises about 0.5 wt % of Vitamin E succinate. In some embodiments, the drug-eluting segment comprises about 0.5 wt % of colloidal silicon dioxide (SiO₂). In some embodiments, the drug-eluting segment comprises about 0.1 wt % of pigment.

In some embodiments, the pigment comprises Aluminum, 4,5-dihydro-5-oxo-1-(4-sulfophenyl)-4-((4-sulfophenyl)azo)-1H-pyrazole-3-carboxylic acid complex, such as FD&C Yellow 5 Aluminum lake, in the amount of about 0.05 wt % of the total weight of the drug-eluting segment and Benzenemethanaminium, N-ethyl-N-(4-((4-(ethyl((3-sulfophenyl)methyl)amino)phenyl)(2-sulfophenyl)methylene)-2,5-cyclohexadi, such as FD&C Blue 1 Aluminum lake, in the amount of 0.05 wt % of the total weight of the drug-eluting segment. FD&C Yellow 5 Aluminum lake and FD&C Blue 1 Aluminum lake are approved food-coloring additives. In some embodiments, the amount of dye in FD&C Yellow 5 Aluminum lake is about 14-16% by weight. In some embodiments, the amount of dye in FD&C Blue 1 Aluminum lake is about 11-13% by weight.

In some embodiments, the drug-eluting segment comprises about 30 wt % to about 40 wt % of risperidone, about 51 wt % to about 61 wt % of PCL, about 2 wt % to about 8 wt % of VA64, about 1 wt % to about 5 wt % of P407, about 0.1 wt % to about 1 wt % of Vitamin E succinate, about 0.1 wt % to about 1 wt % of SiO₂, and about 0.01 wt % to about 0.5 wt % of pigment.

In some embodiments, the drug-eluting segment comprises about 33 wt % to about 37 wt % of risperidone, about 54 wt % to about 58 wt % of PCL, about 4 wt % to about 6 wt % of VA64, about 2 wt % to about 4 wt % of P407, about 0.2 wt % to about 0.8 wt % of Vitamin E succinate, about 0.2 wt % to about 0.8 wt % of SiO₂, and about 0.05 wt % to about 0.15 wt % of pigment.

In some embodiments, the drug-eluting segment comprises about 35.0 wt % of risperidone, about 55.9 wt % of PCL, about 5.0 wt % of VA64, about 3.0 wt % of P407, about 0.5 wt % of Vitamin E succinate, about 0.5 wt % of SiO₂, and about 0.1 wt % of pigment. In some embodiments, the pigment comprises FD&C Yellow 5 Aluminum lake in the amount of about 0.05 wt % of the total weight of the drug-eluting segment and FD&C Blue 1 Aluminum lake in the amount of 0.05 wt % of the total weight of the drug-eluting segment. FD&C Yellow 5 Aluminum lake and FD&C Blue 1 Aluminum lake are approved food-coloring additives. In some embodiments, the amount of dye in FD&C Yellow 5 Aluminum lake is about 14-16% by weight. In some embodiments, the amount of dye in FD&C Blue 1 Aluminum lake is about 11-13% by weight. Exemplary amounts of the components for one embodiment of the carrier polymer-arm segment (drug-eluting segment) are provided in the table below. The amounts are given in approximate weight percent, with the understanding that when ranges are provided, the amounts are chosen so as to add up to 100%. “Pharm. accept. salt” indicates pharmaceutically acceptable salt thereof.

| Carrier polymer-arm | | | |
|---|---|---|---|
| segment (CP-1) | Formulation 1 | Formulation 2 | Formulation 3 |
| risperidone (or | 30-40 | 33-37 | 35.0 |
| pharm. accept. salt) | | | |
| PCL (such as PC17 | 51-61 | 54-58 | 55.9 |
| with viscosity | | | |
| midpoint 1.7 dl/g) | | | |
| VA64 | 2-8 | 4-6 | 5.0 |
| P407 | 1-5 | 2-4 | 3.0 |
| vitE | 0.1-1 | 0.2-0.8 | 0.5 |
| SiO₂ | 0.1-1 | 0.2-0.8 | 0.5 |
| coloring (optional) | 0.01-0.5 | 0.05-0.2 | 0.1 (e.g. 0.05 |
| | | | blue, 0.05 yellow) |

The drug-eluting segments above, while described as being risperidone-formulated, are not limited as such, and can be used with other drugs by replacing part or all of the risperidone component and/or other components, with other drug(s).

In some embodiments, a dosage form for administration of risperidone comprises a gastric residence system comprising about 12 mg to about 60 mg of risperidone. In some embodiments, a dosage form for administration of risperidone comprises a gastric residence system comprising about 12 mg to about 36 mg of risperidone. In some embodiments, a dosage form for administration of risperidone comprises a gastric residence system comprising about 12 mg to about 20 mg of risperidone. In some embodiments, a dosage form for administration of risperidone comprises a gastric residence system comprising about 16 mg of risperidone. In some embodiments, a dosage form for administration of risperidone comprises a gastric residence system comprising about 28 mg to about 36 mg of risperidone. In some embodiments, the dosage form comprises a gastric residence system, wherein the gastric residence system comprises about 32 mg of risperidone. In some embodiments, a dosage form for administration of risperidone comprises a gastric residence system comprising about 44 mg to about 52 mg of risperidone. In some embodiments, the dosage form comprises a gastric residence system, wherein the gastric residence system comprises about 48 mg of risperidone.

In some embodiments, the dosage form comprises a gastric residence system, wherein the gastric residence system comprises a drug-eluting segment comprising about 16 mg of risperidone. In some embodiments, the dosage form comprises a gastric residence system, wherein the gastric residence system comprises a drug-eluting segment comprising about 32 mg of risperidone. In some embodiments, wherein the drug-eluting segment comprises about 30 wt % to about 40 wt % of risperidone, the drug-eluting segment comprises about 51 wt % to about 61 wt % of polycaprolactone (PCL), such as PCL having a viscosity midpoint between about 1.5 dl/g to about 2.1 dl/g, such as Corbion PC17. In some embodiments, the drug-eluting segment comprises about 2 wt % to about 8 wt % of vinylpyrrolidone-vinyl acetate copolymer, such as Kollidon VA64. In some embodiments, the drug-eluting segment comprises about 1 wt % to about 5 wt % of poly(ethylene glycol)-block-poly(propylene glycol)-block-poly(ethylene glycol) polymers, such as H—(OCH2CH2)x-(O—CH(CH3)CH2)y-(OCH2CH2)z-OH where x and z are about 101 and y is about 56, such as Poloxamer 407. In some embodiments, the drug-eluting segment comprises about 0.1 wt % to about 1 wt % of Vitamin E succinate. In some embodiments, the drug-eluting segment comprises about 0.1 wt % to about 1 wt % of colloidal silicon dioxide (SiO₂). In some embodiments, the drug-eluting segment comprises about 0.01 wt % to about 0.5 wt % of pigment.

In some embodiments, wherein the drug-eluting segment comprises about 33 wt % to about 37 wt % of risperidone, the drug-eluting segment comprises about 54 wt % to about 58 wt % of polycaprolactone (PCL), such as PCL having a viscosity midpoint between about 1.5 dl/g to about 2.1 dl/g, such as Corbion PC17. In some embodiments, the drug-eluting segment comprises about 4 wt % to about 6 wt % of vinylpyrrolidone-vinyl acetate copolymer, such as Kollidon VA64. In some embodiments, the drug-eluting segment comprises about 2 wt % to about 4 wt % of poly(ethylene glycol)-block-poly(propylene glycol)-block-poly(ethylene glycol) polymers, such as H—(OCH2CH2)x-(O—CH(CH3)CH2)y-(OCH2CH2)z-OH where x and z are about 101 and y is about 56, such as Poloxamer 407. In some embodiments, the drug-eluting segment comprises about 0.2 wt % to about 0.8 wt % of Vitamin E succinate. In some embodiments, the drug-eluting segment comprises about 0.2 wt % to about 0.8 wt % of colloidal silicon dioxide (SiO₂). In some embodiments, the drug-eluting segment comprises about 0.05 wt % to about 0.2 wt % of pigment.

In some embodiments, wherein the drug-eluting segment comprises about 35 wt % of risperidone, the drug-eluting segment comprises about 55.9 wt % of polycaprolactone (PCL), such as PCL having a viscosity midpoint between about 1.5 dl/g to about 2.1 dl/g, such as Corbion PC17. In some embodiments, the drug-eluting segment comprises about 5.0 wt % of vinylpyrrolidone-vinyl acetate copolymer, such as Kollidon VA64. In some embodiments, the drug-eluting segment comprises about 3.0 wt % of poly(ethylene glycol)-block-poly(propylene glycol)-block-poly(ethylene glycol) polymers, such as H—(OCH2CH2)x-(O—CH(CH3)CH2)y-(OCH2CH2)z-OH where x and z are about 101 and y is about 56, such as Poloxamer 407. In some embodiments, the drug-eluting segment comprises about 0.5 wt % of Vitamin E succinate. In some embodiments, the drug-eluting segment comprises about 0.5 wt % of colloidal silicon dioxide (SiO₂). In some embodiments, the drug-eluting segment comprises about 0.1 wt % of pigment.

In some embodiments, the pigment comprises Aluminum, 4,5-dihydro-5-oxo-1-(4-sulfophenyl)-4-((4-sulfophenyl)azo)-1H-pyrazole-3-carboxylic acid complex, such as FD&C Yellow 5 Aluminum lake, in the amount of about 0.05 wt % of the total weight of the drug-eluting segment and Benzenemethanaminium, N-ethyl-N-(4-((4-(ethyl((3-sulfophenyl)methyl)amino)phenyl)(2-sulfophenyl)methylene)-2,5-cyclohexadi, such as FD&C Blue 1 Aluminum lake, in the amount of 0.05 wt % of the total weight of the drug-eluting segment. FD&C Yellow 5 Aluminum lake and FD&C Blue 1 Aluminum lake are approved food-coloring additives. In some embodiments, the amount of dye in FD&C Yellow 5 Aluminum lake is about 14-16% by weight. In some embodiments, the amount of dye in FD&C Blue 1 Aluminum lake is about 11-13% by weight.

In some embodiments, the drug-eluting segment comprises about 30 wt % to about 40 wt % of risperidone, about 51 wt % to about 61 wt % of PCL, about 2 wt % to about 8 wt % of VA64, about 1 wt % to about 5 wt % of P407, about 0.1 wt % to about 1 wt % of Vitamin E succinate, about 0.1 wt % to about 1 wt % of SiO₂, and about 0.01 wt % to about 0.5 wt % of pigment.

In some embodiments, the drug-eluting segment comprises about 33 wt % to about 37 wt % of risperidone, about 54 wt % to about 58 wt % of PCL, about 4 wt % to about 6 wt % of VA64, about 2 wt % to about 4 wt % of P407, about 0.2 wt % to about 0.8 wt % of Vitamin E succinate, about 0.2 wt % to about 0.8 wt % of SiO₂, and about 0.05 wt % to about 0.15 wt % of pigment.

In some embodiments, the drug-eluting segment comprises about 35.0 wt % of risperidone, about 55.9 wt % of PCL, about 5.0 wt % of VA64, about 3.0 wt % of P407, about 0.5 wt % of Vitamin E succinate, about 0.5 wt % of SiO₂, and about 0.1 wt % of pigment. In some embodiments, the pigment comprises FD&C Yellow 5 Aluminum lake in the amount of about 0.05 wt % of the total weight of the drug-eluting segment and FD&C Blue 1 Aluminum lake in the amount of 0.05 wt % of the total weight of the drug-eluting segment. FD&C Yellow 5 Aluminum lake and FD&C Blue 1 Aluminum lake are approved food-coloring additives. In some embodiments, the amount of dye in FD&C Yellow 5 Aluminum lake is about 14-16% by weight. In some embodiments, the amount of dye in FD&C Blue 1 Aluminum lake is about 11-13% by weight.

Exemplary amounts of the components for one embodiment of the carrier polymer-arm segment (drug-eluting segment) are provided in the table below. The amounts are given in approximate weight percent, with the understanding that when ranges are provided, the amounts are chosen so as to add up to 100%. “Pharm. accept. salt” indicates pharmaceutically acceptable salt thereof.

| Carrier polymer-arm | | | |
|---|---|---|---|
| segment (CP-1) | Formulation 1 | Formulation 2 | Formulation 3 |
| risperidone (or | 30-40 | 33-37 | 35.0 |
| pharm. accept. salt) | | | |
| PCL (such as PC17 | 51-61 | 54-58 | 55.9 |
| with viscosity | | | |
| midpoint 1.7 dl/g) | | | |
| VA64 | 2-8 | 4-6 | 5.0 |
| P407 | 1-5 | 2-4 | 3.0 |
| vitE | 0.1-1 | 0.2-0.8 | 0.5 |
| SiO₂ | 0.1-1 | 0.2-0.8 | 0.5 |
| coloring (optional) | 0.01-0.5 | 0.05-0.2 | 0.1 (e.g. 0.05 |
| | | | blue, 0.05 yellow) |

The drug-eluting segments above, while described as being risperidone-formulated, are not limited as such, and can be used with other drugs by replacing part or all of the risperidone component and/or other components, with other drug(s).

In some embodiments, a stellate-shaped dosage form for administration of risperidone can comprise arms, which arms in turn comprise 1) a carrier polymer-agent arm segment; 2) an inactive arm segment; 3) one or more enteric linkers; 4) one or more time-dependent linkers; 5) release rate-modulating films; and/or 6) other optional spacers. The arms are connected to an elastomeric core in a stellate device arrangement. Typically, six arms are used for a stellate dosage form. In some embodiments, wherein six arms are used for a stellate dosage form, any one of 1, 2, 3, 4, 5, or 6 arms comprise the carrier polymer-agent arm segment. In some embodiments, wherein six arms are used for a stellate dosage form, 3 arms comprise the carrier polymer-agent arm segment. In some embodiments, wherein six arms are used for a stellate dosage form, 6 arms comprise the carrier polymer-agent arm segment.

The carrier polymer-agent arm segments of the risperidone dosage form can comprise risperidone (or a pharmaceutically acceptable salt thereof), polycaprolactone, copovidone (VA64), poloxamer 407 (P407), silica (SiO₂), vitamin E succinate (vitE), and optionally coloring. The calcium salt of risperidone can be used in the carrier polymer-agent arm segment. The polycaprolactone used can be from about 1.5 dL/g to about 1.9 dL/g viscosity, such as about 1.7 dL/g. Any pharmaceutically acceptable coloring agent can be used. Examples of coloring that can be used include FD&C Red 40 Aluminum lake, FD&C Yellow 5 Aluminum lake, or an approximately equal blend of the two. In some embodiments, typically six arms are used for a stellate dosage form, and either 1, 2, 3, 4, 5 or 6 of the arms comprise the carrier polymer-agent arm segment. In some embodiments, 3 of the arms comprise the carrier polymer-agent arm segment. In some embodiments, 6 of the arms comprise the carrier polymer-agent arm segment. In some embodiments, the total amount of agent contained in the dosage form is 1, 2, 3, 4, 5, or 6 times the amount of agent contained in a single arm. In some embodiments, the total amount of agent contained in the dosage form is 3 times the amount of agent contained in a single arm. In some embodiments, the total amount of agent contained in the dosage form is 6 times the amount of agent contained in a single arm. The total amount of weight of risperidone, pharmaceutically acceptable salt of risperidone, or calcium salt of risperidone in the stellate dosage form can range from about 2 mg to about 50 mg, such as about 4 mg to about 30 mg, or about 10 mg to about 20 mg, or about 20 mg to about 30 mg, or about 25 mg to about 35 mg, or about 12 mg to about 16 mg, or about 26 mg to about 30 mg, or about 3 mg to about 5 mg, or about 8 mg to about 10 mg, or about 13 mg to about 15 mg, or about 17 mg to about 20 mg, or about 22 mg to about 24 mg, or about 27 mg to about 29 mg. In some embodiments, the total amount of weight of risperidone, pharmaceutically acceptable salt of risperidone, or calcium salt of risperidone in the stellate dosage form is about 14 mg, or about 28 mg.

The inactive arm segments of the risperidone dosage form can comprise polycaprolactone (PCL), a radiopaque substance, and optionally coloring. The polycaprolactone used can be from about 1.5 dL/g to about 1.9 dL/g viscosity, such as about 1.7 dL/g. The radiopaque substance can be (BiO)₂CO₃. Any pharmaceutically acceptable coloring agent can be used. An example of coloring that can be used includes FD&C Blue #5.

The enteric disintegrating matrices of the risperidone dosage form can comprise polycaprolactone (PCL), hydroxypropyl methyl cellulose acetate succinate (HPMCAS), poloxamer 407 (P407), and optionally coloring. The polycaprolactone used can be from about 1.5 dL/g to about 1.9 dL/g viscosity, such as about 1.7 dL/g. The HPMCAS used can be MG grade (M grade: about 7-11% acetyl content, about 10-14% succinoyl content, about 21-25% methoxyl content, about 5-9% hydroxypropoxy content; G grade: granular). Any pharmaceutically acceptable coloring agent can be used. An example of coloring that can be used includes ferrosoferric oxide.

The time dependent disintegrating matrices of the risperidone dosage form can comprise poly(D,L-lactide-co-glycolide) (PLGA), polyethylene oxide (PEO), and optionally coloring. The poly(D,L-lactide-co-glycolide) can be in about a 75:25 lactide:glycolide molar ratio with a viscosity range of about 0.32-0.44 dL/g. The polyethylene oxide used can be from about 60,000 MW to about 125,000 MW, such as about 90,000 MW to 110,000 MW, or about 100,000 MW.

The time dependent disintegrating matrices of the risperidone dosage form can comprise polycaprolactone (PCL), poly(D,L-lactide-co-glycolide) (PLGA), polyethylene oxide (PEO), and optionally coloring. The PCL can have a viscosity midpoint between about 1.5 dl/g to about 2.1 dl/g; such as 1.7 dl/g, such as Corbion PC17. The poly(D,L-lactide-co-glycolide) can be in about a 50:50 lactide:glycolide molar ratio with a viscosity range of about 0.32-0.44 dL/g. The polyethylene oxide used can be from about 60,000 MW to about 125,000 MW, such as about 90,000 MW to 110,000 MW, or about 100,000 MW.

The release rate-modulating film of the risperidone dosage form can comprise polycaprolactone (PCL), copovidone (such as VA64) and magnesium stearate. The polycaprolactone used can be from about 1.5 dL/g to about 1.9 dL/g viscosity, such as about 1.7 dL/g.

The central elastomer of the risperidone dosage form can be of about 40 A to about 60 A durometer, such as about 45 A to about 55 A durometer, or about 50 A durometer. The central elastomer can be made from liquid silicone rubber; e.g., the central elastomer can comprise cured liquid silicone rubber.

Exemplary amounts for the various components of the risperidone dosage form are provided in the table below. The amounts are given in approximate weight percent, with the understanding that when ranges are provided, the amounts are chosen so as to add up to 100%.

| | Formulation 1 | Formulation 2 | Formulation 3 |
|---|---|---|---|
| Carrier polymer- | | | |
| arm segment | | | |
| risperidone (or | 30-40 | 33-37 | 35.0 |
| pharm. accept. salt) | | | |
| PCL (such as PC17 | 51-61 | 54-58 | 55.9 |
| with viscosity | | | |
| midpoint 1.7 dl/g) | | | |
| VA64 | 2-8 | 4-6 | 5.0 |
| P407 | 1-5 | 2-4 | 3.0 |
| vitE | 0.1-1 | 0.2-0.8 | 0.5 |
| SiO₂ | 0.1-1 | 0.2-0.8 | 0.5 |
| coloring (optional) | 0.01-0.5 | 0.05-0.2 | 0.1 (e.g. 0.05 |
| | | | blue, 0.05 yellow) |
| Inactive spacer | | | |
| PCL (such as PC17 | 61-71 | 64-69 | 66.45 |
| with viscosity | | | |
| midpoint 1.7 dl/g) | | | |
| VA64 | 27-37 | 30-34 | 32 |
| P407 | 0.2-4 | 0.5-2.5 | 1.5 |
| coloring (optional) | 0.005-0.2 | 0.01-0.1 | 0.05 (e.g. Blue #1) |
| Enteric disintegrating matrix | | | |
| PCL (such as PC17 | 30-40 | 32-37 | 33.95 |
| with viscosity | | | |
| midpoint 1.7 dl/g) | | | |
| HPMCAS | 60-70 | 62-66 | 63.95 |
| P407 | 0.5-5 | 1-3 | 2 |
| coloring (optional) | 0.01-0.5 | 0.05-0.15 | 0.1 (e.g. E172) |
| Time-dependent disintegrating matrix | | | |
| PCL (viscosity | 40-50 | 43-47 | 44.95 |
| midpoint 1.7 dl/g) | | | |
| PDLG5004A | 30-40 | 33-37 | 35 |
| PDLG5004 | 10-25 | 15-20 | 18 |
| PEO(100k) | 0.5-5 | 1-3 | 2 |
| coloring (optional) | 0.005-0.2 | 0.01-0.1 | 0.05 (e.g. E172) |
| ODMTEP disintegrating matrix | | | |
| PCL (such as PC17 | 25-35 | 28-32 | 30 |
| with viscosity | | | |
| midpoint 1.7 dl/g) | | | |
| HPMCAS | 60-70 | 63-67 | 64.9 |
| Stearic acid | 1-5 | 2-3 | 2.5 |
| Propylene Glycol | 1-5 | 2-3 | 2.5 |
| Release rate- | | | |
| modulating film | | | |
| PCL (such as PC17 | 67-77 | 71-76 | 73.5 |
| with viscosity | | | |
| midpoint 1.7 dl/g) | | | |
| VA64 | 20-30 | 22-27 | 24.5 |
| Mg stearate | 0.5-5 | 1-3 | 2.0 |

The assembled arms can comprise 1) a first inert segment; 2) a first disintegrating matrix segment; 3) a second inert segment; 4) a second disintegrating matrix segment; 5) a third inert segment; 6) a fourth inert segment; 7) the drug eluting segment, wherein the drug eluting segment comprises a carrier polymer, and risperidone or a salt thereof, and wherein the drug eluting segment further comprises a coating comprising a release rate-modulating polymer film; 8) an optional fifth inert segment; and 9) a third disintegrating matrix segment, which can be arranged in various orders. One such order is, starting from the proximal end which is attached to the central elastomer, and proceeding to the distal end: (a first inert segment) (a first disintegrating matrix segment) (a second inert segment) (a second disintegrating matrix segment) (a third inert segment) (a fourth inert segment) (a drug eluting segment) (an optional fifth inert segment) (a third disintegrating matrix segment). In some embodiments, the fourth inert segment is an inactive spacer. In some embodiments, the first, second, third and the optional fifth inert segments are rPCL spacers. Optional rPCL spacers (inert segments) of about 0.2-2 mm length, such as about 0.5 mm length, can be inserted between any two components above, or added to the outer tip of the assembled arm, or between the inner tip of the assembled arm and the elastomeric core.

Approximate dimensions for the length of the segments on an exemplary drug-eluting arm are provided below.

### Drug-Eluting Arm

| | Dimension | | Dimension | | Dimension | |
|---|---|---|---|---|---|---|
| Component | set 1 | | set 2 | | set 3 | |
| Carrier | 1-6 | mm | 2-3 | mm | 2.4 | mm |
| polymer- | | | | | | |
| agent segment | | | | | | |
| Inactive | 2-9 | mm | 4-7 | mm | 5.6 | mm |
| segment | | | | | | |
| (Fourth inert | | | | | | |
| segment) | | | | | | |
| Inert segment | 0.1-2 | mm | 0.25-1 | mm | 0.5 | mm |
| (First, second, | | | | | | |
| third, fifth) | | | | | | |
| Enteric | 0.5-5 | mm | 1-3 | mm | 1.85 | mm |
| disintegrating | | | | | | |
| matrix | | | | | | |
| Timed | 0.25-5 | mm | 0.5-2 | mm | 1.0 | mm |
| disintegrating | | | | | | |
| matrix | | | | | | |
| Third | 1-6 | mm | 3-5 | mm | 4 | mm |
| disintegrating | | | | | | |
| matrix | | | | | | |

Approximate dimensions for the length and thickness of the segments on an exemplary drug-eluting arm are provided below.

| | Dimension | Dimension | Dimension |
|---|---|---|---|
| Component | set 1 | set 2 | set 3 |
| Carrier | 1-6 mm length | 2-3 mm length | 2.4 mm length |
| polymer- | 3.0-3.7 mm | 3.2-3.4 mm | 3.3 mm |
| agent segment | thickness | thickness | thickness |
| Inactive segment | 2-9mm length | 4-7 mm length | 5.6 mm length |
| (Fourth inert | 3.0-3.7 mm | 3.2-3.4 mm | 3.3 mm |
| segment) | thickness | thickness | thickness |
| Inert segment | 0.1-2 mm length | 0.25-1 mm length | 0.5 mm length |
| (First, second, | 3.0-3.7 mm | 3.2-3.4 mm | 3.3 mm |
| third, fifth) | thickness | thickness | thickness |
| Enteric | 0.5-5 mm length | 1-3 mm length | 1.85 mm length |
| disintegrating | 3.0-3.7 mm | 3.2-3.4 mm | 3.3 mm |
| matrix | thickness | thickness | thickness |
| Timed | 0.25-5 mm length | 0.5-2 mm length | 1.0 mm length |
| disintegrating | 3.0-3.7 mm | 3.2-3.4 mm | 3.3 mm |
| matrix | thickness | thickness | thickness |
| Third | 1-6 mm length | 3-5 mm length | 4 mm length |
| disintegrating | 3.0-3.7 mm | 3.2-3.4 mm | 3.3 mm |
| matrix | thickness | thickness | thickness |

Approximate dimensions for the length and thickness of the segments on an exemplary drug-eluting arm are provided below.

### Drug-Eluting Arm

| | Dimension | Dimension | Dimension |
|---|---|---|---|
| Component | set 1 | set 2 | set 3 |
| Carrier | 1-6 mm length | 2-3 mm length | 2.4 mm length |
| polymer- | 3.0-3.7 mm | 3.2-3.4 mm | 3.3 mm |
| agent segment | thickness | thickness | thickness |
| Inactive segment | 2-9mm length | 4-7 mm length | 5.6 mm length |
| (Fourth inert | 3.0-3.7 mm | 3.2-3.4 mm | 3.3 mm |
| segment) | thickness | thickness | thickness |
| Inert segment | 0.1-2 mm length | 0.25-1 mm length | 0.5 mm length |
| (First, second, | 3.0-3.7 mm | 3.2-3.4 mm | 3.3 mm |
| third, fifth) | thickness | thickness | thickness |
| Enteric | 0.5-5 mm length | 1-3 mm length | 1.85 mm length |
| disintegrating | 3.0-3.7 mm | 3.2-3.4 mm | 3.3 mm |
| matrix | thickness | thickness | thickness |
| Timed | 0.25-5 mm length | 0.5-2 mm length | 1.0 mm length |
| disintegrating | 3.0-3.7 mm | 3.2-3.4 mm | 3.3 mm |
| matrix | thickness | thickness | thickness |
| Third | 1-6 mm length | 3-5 mm length | 4 mm length |
| disintegrating | 2.8-3.5 mm | 3.0-3.2 mm | 3.1 mm |
| matrix | thickness | thickness | thickness |

The assembled arms can comprise 1) a first inert segment; 2) a first disintegrating matrix segment; 3) a second inert segment; 4) a second disintegrating matrix segment; 5) a third inert segment; 6) a fourth inert segment; 7) an optional fifth inert segment; and 8) a third disintegrating matrix segment, and can be arranged in various orders. One such order is, starting from the proximal end which is attached to the central elastomer, and proceeding to the distal end: (a first inert segment) (a first disintegrating matrix segment) (a second inert segment) (a second disintegrating matrix segment) (a third inert segment) (a fourth inert segment) (an optional fifth inert segment) (a third disintegrating matrix segment). Approximate dimensions for the length of the segments on each arm are provided below. Optional rPCL spacers (inert segments) of about 0.2-2 mm length, such as about 0.5 mm length, can be inserted between any two components of the arm, or added to the outer tip of the assembled arm, or between the inner tip of the assembled arm and the elastomeric core. It will be appreciated that this embodiment of the assembled arm lacks a drug-eluting segment, and can be used when it is desired to use one or more non-drug-eluting arms for the risperidone dosage form.

Approximate dimensions for the length of the segments on an exemplary non-drug-eluting arm are provided below:

### Non-Drug-Eluting-Arm

| Component | Dimension set 1 | | Dimension set 2 | | Dimension set 3 | |
|---|---|---|---|---|---|---|
| Inactive | 5-12 | mm | 7-9 | mm | 8 | mm |
| segment | | | | | | |
| (Fourth inert | | | | | | |
| segment) | | | | | | |
| Inert segment | 0.1-2 | mm | 0.25-1 | mm | 0.5 | mm |
| (First, second, | | | | | | |
| third, fifth) | | | | | | |
| Enteric | 0.5-5 | mm | 1-3 | mm | 1.85 | mm |
| disintegrating | | | | | | |
| matrix | | | | | | |
| Timed | 0.25-5 | mm | 0.5-2 | mm | 1.0 | mm |
| disintegrating | | | | | | |
| matrix | | | | | | |
| Third | 1-6 | mm | 3-5 | mm | 4 | mm |
| disintegrating | | | | | | |
| matrix | | | | | | |

Approximate dimensions for the length and thickness of the segments on an exemplary non-drug-eluting arm are provided below:

### Non-Drug-Eluting-Arm

| | Dimension | Dimension | Dimension |
|---|---|---|---|
| Component | set 1 | set 2 | set 3 |
| Inactive segment | 5-12 mm length | 7-9 mm length | 8 mm length |
| (Fourth inert | 3.0-3.7 mm | 3.2-3.4 mm | 3.3 mm |
| segment) | thickness | thickness | thickness |
| Inert segment | 0.1-2 mm length | 0.25-1 mm length | 0.5 mm length |
| (First, second, | 3.0-3.7 mm | 3.2-3.4 mm | 3.3 mm |
| third, fifth) | thickness | thickness | thickness |
| Enteric | 0.5-5 mm length | 1-3 mm length | 1.85 mm length |
| disintegrating | 3.0-3.7 mm | 3.2-3.4 mm | 3.3 mm |
| matrix | thickness | thickness | thickness |
| Timed | 0.25-5 mm length | 0.5-2 mm length | 1.0 mm length |
| disintegrating | 3.0-3.7 mm | 3.2-3.4 mm | 3.3 mm |
| matrix | thickness | thickness | thickness |
| Third | 1-6 mm length | 3-5 mm length | 4 mm length |
| disintegrating | 3.0-3.7 mm | 3.2-3.4 mm | 3.3 mm |
| matrix | thickness | thickness | thickness |

Approximate dimensions for the length and thickness of the segments on an exemplary non-drug-eluting arm are provided below:

### Non-Drug-Eluting-Arm

| | Dimension | Dimension | Dimension |
|---|---|---|---|
| Component | set 1 | set 2 | set 3 |
| Inactive segment | 5-12 mm length | 7-9 mm length | 8 mm length |
| (Fourth inert | 3.0-3.7 mm | 3.2-3.4 mm | 3.3 mm |
| segment) | thickness | thickness | thickness |
| Inert segment | 0.1-2 mm length | 0.25-1 mm length | 0.5 mm length |
| (First, second, | 3.0-3.7 mm | 3.2-3.4 mm | 3.3 mm |
| third, fifth) | thickness | thickness | thickness |
| Enteric | 0.5-5 mm length | 1-3 mm length | 1.85 mm length |
| disintegrating | 3.0-3.7 mm | 3.2-3.4 mm | 3.3 mm |
| matrix | thickness | thickness | thickness |
| Timed | 0.25-5 mm length | 0.5-2 mm length | 1.0 mm length |
| disintegrating | 3.0-3.7 mm | 3.2-3.4 mm | 3.3 mm |
| matrix | thickness | thickness | thickness |
| Third | 1-6 mm length | 3-5 mm length | 4 mm length |
| disintegrating | 2.8-3.5 mm | 3.0-3.2 mm | 3.1 mm |
| matrix | thickness | thickness | thickness |

Approximate dimensions for the length of the segments on an exemplary drug-eluting arm are provided below

### Drug-Eluting Arm

| Component | Dimension set 1 | | Dimension set 2 | | Dimension set 3 | |
|---|---|---|---|---|---|---|
| Carrier | 1-6 | mm | 2-3 | mm | 2.4 | mm |
| polymer- | | | | | | |
| agent segment | | | | | | |
| Inactive | 5-12 | mm | 7-9 | mm | 8 | mm |
| segment | | | | | | |
| (Fourth inert | | | | | | |
| segment) | | | | | | |
| Inert segment | 0.1-2 | mm | 0.25-1 | mm | 0.5 | mm |
| (First, second, | | | | | | |
| third, fifth) | | | | | | |
| Enteric | 0.5-5 | mm | 1-3 | mm | 1.85 | mm |
| disintegrating | | | | | | |
| matrix | | | | | | |
| Timed | 0.25-5 | mm | 0.5-2 | mm | 1.0 | mm |
| disintegrating | | | | | | |
| matrix | | | | | | |
| Third | 1-6 | mm | 3-5 | mm | 4 | mm |
| disintegrating | | | | | | |
| matrix | | | | | | |

The gastric residence systems or dosage forms above, while described as being risperidone-formulated, are not limited as such, and can be used with other drugs by replacing the segment(s) containing risperidone and/or replacing inert segment(s), with segments containing other drugs.

Exemplary amounts for the various components of the risperidone dosage form are provided in the table below. The amounts are given in approximate weight percent, with the understanding that when ranges are provided, the amounts are chosen so as to add up to 100%.

| | Formulation 1 | Formulation 2 | Formulation 3 |
|---|---|---|---|
| Carrier polymer- | | | |
| arm segment | | | |
| risperidone (or | 30-40 | 33-37 | 35.0 |
| pharm. accept. salt) | | | |
| PCL (such as PC17 | 51-61 | 54-58 | 55.9 |
| with viscosity | | | |
| midpoint 1.7 dl/g) | | | |
| VA64 | 2-8 | 4-6 | 5.0 |
| P407 | 1-5 | 2-4 | 3.0 |
| vitE | 0.1-1 | 0.2-0.8 | 0.5 |
| SiO₂ | 0.1-1 | 0.2-0.8 | 0.5 |
| coloring (optional) | 0.01-0.5 | 0.05-0.2 | 0.1 (e.g. 0.05 |
| | | | blue, 0.05 yellow) |
| Inactive spacer (IA47) | | | |
| PCL (such as PC17 | 61-71 | 64-69 | 66.45 |
| with viscosity | | | |
| midpoint 1.7 dl/g) | | | |
| VA64 | 27-37 | 30-34 | 32 |
| P407 | 0.2-4 | 0.5-2.5 | 1.5 |
| coloring (optional) | 0.005-0.2 | 0.01-0.1 | 0.05 (e.g. Blue #1) |
| Enteric disintegrating matrix | | | |
| PCL (such as PC17 | 30-40 | 32-37 | 33.95 |
| with viscosity | | | |
| midpoint 1.7 dl/g) | | | |
| HPMCAS | 60-70 | 62-66 | 63.95 |
| P407 | 0.5-5 | 1-3 | 2 |
| coloring (optional) | 0.01-0.5 | 0.05-0.15 | 0.1 (e.g. E172) |
| Time-dependent disintegrating matrix | | | |
| PCL (viscosity | 40-50 | 43-47 | 44.95 |
| midpoint 1.7 dl/g) | | | |
| PDLG5004A | 30-40 | 33-37 | 35 |
| PDLG5004 | 10-25 | 15-20 | 18 |
| PEO(100k) | 0.5-5 | 1-3 | 2 |
| coloring (optional) | 0.005-0.2 | 0.01-0.1 | 0.05 (e.g. E172) |
| Inactive Spacer (IA36) | | | |
| PCL (such as PC17 | 35-45 | 38-42 | 39.995 |
| with viscosity | | | |
| midpoint 1.7 dl/g) | | | |
| VA64 | 37-47 | 40-44 | 42 |
| PEO100k | 10-20 | 13-17 | 15 |
| P407 | 1-5 | 2-4 | 3 |
| Release rate- | | | |
| modulating film | | | |
| PCL (such as PC17 | 67-77 | 71-76 | 73.5 |
| with viscosity | | | |
| midpoint 1.7 dl/g) | | | |
| VA64 | 20-30 | 22-27 | 24.5 |
| Mg stearate | 0.5-5 | 1-3 | 2.0 |

The assembled arms can comprise 1) a first inert segment; 2) a first disintegrating matrix segment; 3) a second inert segment; 4) a second disintegrating matrix segment; 5) a third inert segment; 6) a fourth inert segment; 7) a drug eluting segment, wherein the drug eluting segment comprises a carrier polymer, and risperidone or a salt thereof, and wherein the drug eluting segment further comprises a coating comprising a release rate-modulating polymer film; 8) an optional sixth inert segment; and 9) a fifth inert segment, which can be arranged in various orders. One such order is, starting from the proximal end which is attached to the central elastomer, and proceeding to the distal end: (a first inert segment) (a first disintegrating matrix segment) (a second inert segment) (a second disintegrating matrix segment) (a third inert segment) (a fourth inert segment) (a drug eluting segment) (an optional sixth inert segment) (a fifth inert segment). In some embodiments, the fourth inert segment is an inactive spacer. In some embodiments, the first, second, third and the optional sixth inert segments are rPCL spacers. In some embodiments, the fifth inert segment is an inactive spacer. Optional rPCL spacers (inert segments) of about 0.2-2 mm length, such as about 0.5 mm length, can be inserted between any two components above, or added to the outer tip of the assembled arm, or between the inner tip of the assembled arm and the elastomeric core.

Approximate dimensions for the length of the segments on an exemplary drug-eluting arm are provided below.

| Component | Dimension set 1 | | Dimension set 2 | | Dimension set 3 | |
|---|---|---|---|---|---|---|
| Carrier | 1-6 | mm | 2-3 | mm | 2.4 | mm |
| polymer- | | | | | | |
| agent segment | | | | | | |
| Inactive | 2-9 | mm | 4-7 | mm | 5.6 | mm |
| segment | | | | | | |
| (Fourth inert | | | | | | |
| segment) | | | | | | |
| Inert segment | 0.1-2 | mm | 0.25-1 | mm | 0.5 | mm |
| (First, second, | | | | | | |
| third, sixth) | | | | | | |
| Enteric | 0.5-5 | mm | 1-3 | mm | 1.85 | mm |
| disintegrating | | | | | | |
| matrix | | | | | | |
| Timed | 0.25-5 | mm | 0.5-2 | mm | 1.0 | mm |
| disintegrating | | | | | | |
| matrix | | | | | | |
| Inactive | 1-6 | mm | 3-5 | mm | 4 | mm |
| segment | | | | | | |
| (Fifth inert | | | | | | |
| segment) | | | | | | |

Approximate dimensions for the length and thickness of the segments on an exemplary drug-eluting arm are provided below.

### Drug-Eluting Arm

| | Dimension | Dimension | Dimension |
|---|---|---|---|
| Component | set 1 | set 2 | set 3 |
| Carrier polymer- | 1-6 mm length | 2-3 mm length | 2.4 mm length |
| agent segment | 3.0-3.7 mm | 3.2-3.4 mm | 3.3 mm |
| | thickness | thickness | thickness |
| Inactive | 2-9 mm length | 4-7 mm length | 5.6 mm length |
| segment | 3.0-3.7 mm | 3.2-3.4 mm | 3.3 mm |
| (Fourth inert | thickness | thickness | thickness |
| segment) | | | |
| Inert segment | 0.1-2 mm length | 0.25-1 mm length | 0.5 mm length |
| (First, second, | 3.0-3.7 mm | 3.2-3.4 mm | 3.3 mm |
| third, sixth) | thickness | thickness | thickness |
| Enteric | 0.5-5 mm length | 1-3 mm length | 1.85 mm length |
| disintegrating | 3.0-3.7 mm | 3.2-3.4 mm | 3.3 mm |
| matrix | thickness | thickness | thickness |
| Timed | 0.25-5 mm length | 0.5-2 mm length | 1.0 mm length |
| disintegrating | 3.0-3.7 mm | 3.2-3.4 mm | 3.3 mm |
| matrix | thickness | thickness | thickness |
| Inactive segment | 1-6 mm length | 3-5 mm length | 4 mm length |
| (Fifth inert | 3.0-3.7 mm | 3.2-3.4 mm | 3.3 mm |
| segment) | thickness | thickness | thickness |

Approximate dimensions for the length and thickness of the segments on an exemplary drug-eluting arm are provided below.

| | Dimension | Dimension | Dimension |
|---|---|---|---|
| Component | set 1 | set 2 | set 3 |
| Carrier polymer- | 1-6 mm length | 2-3 mm length | 2.4 mm length |
| agent segment | 3.0-3.7 mm | 3.2-3.4 mm | 3.3 mm |
| | thickness | thickness | thickness |
| Inactive segment | 2-9 mm length | 4-7 mm length | 5.6 mm length |
| (Fourth inert | 3.0-3.7 mm | 3.2-3.4 mm | 3.3 mm |
| segment) | thickness | thickness | thickness |
| Inert segment | 0.1-2 mm length | 0.25-1 mm length | 0.5 mm length |
| (First, second, | | | |
| third, sixth) | 3.0-3.7 mm | 3.2-3.4 mm | 3.3 mm |
| | thickness | thickness | thickness |
| Enteric | 0.5-5 mm length | 1-3 mm length | 1.85 mm length |
| disintegrating | | | |
| matrix | 3.0-3.7 mm | 3.2-3.4 mm | 3.3 mm |
| | thickness | thickness | thickness |
| Timed | 0.25-5 mm length | 0.5-2 mm length | 1.0 mm length |
| disintegrating | | | |
| matrix | 3.0-3.7 mm | 3.2-3.4 mm | 3.3 mm |
| | thickness | thickness | thickness |
| Inactive segment | 1-6 mm length | 3-5 mm length | 4 mm length |
| (Fifth inert | 2.8-3.5 mm | 3.0-3.2 mm | 3.1 mm |
| segment) | thickness | thickness | thickness |

The assembled arms can comprise 1) a first inert segment; 2) a first disintegrating matrix segment; 3) a second inert segment; 4) a second disintegrating matrix segment; 5) a third inert segment; 6) a fourth inert segment; 7) an optional sixth inert segment; and 8) a fifth inert segment, and can be arranged in various orders. One such order is, starting from the proximal end which is attached to the central elastomer, and proceeding to the distal end: (a first inert segment) (a first disintegrating matrix segment) (a second inert segment) (a second disintegrating matrix segment) (a third inert segment) (a fourth inert segment) (an optional sixth inert segment) (a fifth inert segment). Approximate dimensions for the length of the segments on each arm are provided below. Optional rPCL spacers (inert segments) of about 0.2-2 mm length, such as about 0.5 mm length, can be inserted between any two components above, or added to the outer tip of the assembled arm, or between the inner tip of the assembled arm and the elastomeric core. In some embodiments, the fourth inert segment is an inactive spacer. In some embodiments, the first, second, third and the optional sixth inert segments are rPCL spacers. In some embodiments, the fifth inert segment is an inactive spacer. It will be appreciated that this embodiment of the assembled arm lacks a drug-eluting segment, and can be used when it is desired to use one or more non-drug-eluting arms for the risperidone dosage form.

Approximate dimensions for the length of the segments on an exemplary non-drug-eluting arm are provided below:

### Non-Drug-Eluting-Arm

| Component | Dimension set 1 | | Dimension set 2 | | Dimension set 3 | |
|---|---|---|---|---|---|---|
| Inactive | 5-12 | mm | 7-9 | mm | 8 | mm |
| segment | | | | | | |
| (Fourth inert | | | | | | |
| segment) | | | | | | |
| Inert segment | 0.1-2 | mm | 0.25-1 | mm | 0.5 | mm |
| (First, second, | | | | | | |
| third, sixth) | | | | | | |
| Enteric | 0.5-5 | mm | 1-3 | mm | 1.85 | mm |
| disintegrating | | | | | | |
| matrix | | | | | | |
| Timed | 0.25-5 | mm | 0.5-2 | mm | 1.0 | mm |
| disintegrating | | | | | | |
| matrix | | | | | | |
| Inactive | 1-6 | mm | 3-5 | mm | 4 | mm |
| segment | | | | | | |
| (Fifth inert | | | | | | |
| segment) | | | | | | |

The gastric residence systems or dosage forms above, while described as being risperidone-formulated, are not limited as such, and can be used with other drugs by replacing the segment(s) containing risperidone and/or replacing inert segment(s), with segments containing other drugs.

In some embodiments, a stellate-shaped dosage form for administration of risperidone can comprise arms, which arms in turn comprise 1) a carrier polymer-agent arm segment; 2) an inactive arm segment; 3) one or more enteric linkers; 4) one or more time-dependent linkers; 5) release rate-modulating films; and/or 6) other optional spacers. The arms are connected to an elastomeric core in a stellate device arrangement. Typically, six arms are used for a stellate dosage form. In some embodiments, wherein six arms are used for a stellate dosage form, any one of 1, 2, 3, 4, 5, or 6 arms comprise the carrier polymer-agent arm segment. In some embodiments, wherein six arms are used for a stellate dosage form, 1 arm comprises the carrier polymer-agent arm segment. In some embodiments, wherein six arms are used for a stellate dosage form, 2 arms comprise the carrier polymer-agent arm segment. In some embodiments, wherein six arms are used for a stellate dosage form, 3 arms comprise the carrier polymer-agent arm segment. In some embodiments, wherein six arms are used for a stellate dosage form, 6 arms comprise the carrier polymer-agent arm segment.

The carrier polymer-agent arm segments of the risperidone dosage form can comprise risperidone (or a pharmaceutically acceptable salt thereof), polycaprolactone, copovidone (VA64), poloxamer 407 (P407), silica (SiO₂), vitamin E succinate (vitE), and optionally coloring. The calcium salt of risperidone can be used in the carrier polymer-agent arm segment. The polycaprolactone used can be from about 1.5 dL/g to about 1.9 dL/g viscosity, such as about 1.7 dL/g. Any pharmaceutically acceptable coloring agent can be used. Examples of coloring that can be used include FD&C Red 40 Aluminum lake, FD&C Yellow 5 Aluminum lake, or an approximately equal blend of the two. In some embodiments, typically six arms are used for a stellate dosage form, and either 1, 2, 3, 4, 5 or 6 of the arms comprise the carrier polymer-agent arm segment. In some embodiments, 1 of the arms comprises the carrier polymer-agent arm segment. In some embodiments, 2 of the arms comprise the carrier polymer-agent arm segment. In some embodiments, 3 of the arms comprise the carrier polymer-agent arm segment. In some embodiments, 6 of the arms comprise the carrier polymer-agent arm segment. In some embodiments, the total amount of agent contained in the dosage form is 1, 2, 3, 4, 5, or 6 times the amount of agent contained in a single arm. In some embodiments, the total amount of agent contained in the dosage form is same as the amount of agent contained in a single arm. In some embodiments, the total amount of agent contained in the dosage form is 3 times the amount of agent contained in a single arm. In some embodiments, the total amount of agent contained in the dosage form is 6 times the amount of agent contained in a single arm. The total amount of weight of risperidone, pharmaceutically acceptable salt of risperidone, or calcium salt of risperidone in the stellate dosage form can range from about 2 mg to about 60 mg, such as about 4 mg to about 50 mg, or about 10 mg to about 20 mg, or about 20 mg to about 30 mg, or about 25 mg to about 35 mg, or about 14 mg to about 18 mg, or about 30 mg to about 34 mg, about 46 mg to about 50 mg, or about 3 mg to about 5 mg, or about 8 mg to about 10 mg, or about 13 mg to about 15 mg, or about 15 mg to about 17 mg, or about 17 mg to about 20 mg, or about 22 mg to about 24 mg, or about 27 mg to about 29 mg, or about 31 mg to about 33 mg, or about 47 mg to about 49 mg. In some embodiments, the total amount of weight of risperidone, pharmaceutically acceptable salt of risperidone, or calcium salt of risperidone in the stellate dosage form is about 16 mg, about 32 mg or about 48 mg.

The inactive arm segments of the risperidone dosage form can comprise polycaprolactone (PCL), a radiopaque substance, and optionally coloring. The polycaprolactone used can be from about 1.5 dL/g to about 1.9 dL/g viscosity, such as about 1.7 dL/g. The radiopaque substance can be (BiO)₂CO₃. Any pharmaceutically acceptable coloring agent can be used. An example of coloring that can be used includes FD&C Blue #5.

The enteric disintegrating matrices of the risperidone dosage form can comprise polycaprolactone (PCL), hydroxypropyl methyl cellulose acetate succinate (HPMCAS), and poloxamer 407 (P407). The polycaprolactone used can be from about 1.5 dL/g to about 1.9 dL/g viscosity, such as about 1.7 dL/g. The HPMCAS used can be MG grade (M grade: about 7-11% acetyl content, about 10-14% succinoyl content, about 21-25% methoxyl content, about 5-9% hydroxypropoxy content; G grade: granular).

In some embodiments, a filament is wrapped circumferentially around a gastric residence system (e.g. by connecting the distal ends of each arm). The filament circumferentially wrapped around a gastric residence system and connecting one or more the arms of the risperidone dosage form can be a disintegrating filament. In some embodiments, the filament comprise poly (lactic-co-glycolic acid) and/or polyglycolic acid.

The time dependent disintegrating matrices of the risperidone dosage form can comprise polycaprolactone (PCL), poly(D,L-lactide-co-glycolide) (PLGA), polyethylene oxide (PEO), and optionally coloring. The PCL can have a viscosity midpoint between about 1.0 dl/g to about 1.4 dl/g; such as 1.2 dl/g, such as Corbion PC12. The poly(D,L-lactide-co-glycolide) can be in about a 50:50 lactide:glycolide molar ratio with a viscosity range of about 0.32-0.44 dL/g. The polyethylene oxide used can be from about 60,000 MW to about 125,000 MW, such as about 90,000 MW to 110,000 MW, or about 100,000 MW.

The release rate-modulating film of the risperidone dosage form can comprise polycaprolactone (PCL), copovidone (such as VA64) and magnesium stearate. The polycaprolactone used can be from about 1.5 dL/g to about 1.9 dL/g viscosity, such as about 1.7 dL/g.

The central elastomer of the risperidone dosage form can be of about 40 A to about 60 A durometer, such as about 45 A to about 55 A durometer, or about 50 A durometer. The central elastomer can be made from liquid silicone rubber; e.g., the central elastomer can comprise cured liquid silicone rubber.

In one embodiment, exemplary amounts for the various components of the risperidone dosage form are provided in the tables below. The amounts are given in approximate weight percent, with the understanding that when ranges are provided, the amounts are chosen so as to add up to 100%.

| Carrier polymer- | | | |
|---|---|---|---|
| arm segment | Formulation 1 | Formulation 2 | Formulation 3 |
| risperidone (or | 30-40 | 33-37 | 35.0 |
| pharm. accept. salt) | | | |
| PCL (such as PC17 | 51-61 | 54-58 | 55.9 |
| with viscosity | | | |
| midpoint 1.7 dl/g) | | | |
| VA64 | 2-8 | 4-6 | 5.0 |
| P407 | 1-5 | 2-4 | 3.0 |
| vitE | 0.1-1 | 0.2-0.8 | 0.5 |
| SiO₂ | 0.1-1 | 0.2-0.8 | 0.5 |
| coloring | 0.01-0.5 | 0.05-0.2 | 0.1 (e.g. 0.05 |
| (optional) | | | blue, 0.05 yellow) |

| Inactive spacer | Formulation 1 | Formulation 2 | Formulation 3 |
|---|---|---|---|
| PCL (such as PC17 | 61-71 | 64-69 | 66.45 |
| with viscosity | | | |
| midpoint 1.7 dl/g) | | | |
| VA64 | 27-37 | 30-34 | 32 |
| P407 | 0.2-4 | 0.5-2.5 | 1.5 |
| coloring | 0.005-0.2 | 0.01-0.1 | 0.05 (e.g. |
| (optional) | | | Blue #1) |

| Enteric | | | |
|---|---|---|---|
| disintegrating | | | |
| matrix | Formulation 1 | Formulation 2 | Formulation 3 |
| PCL (such as PC17 | 30-40 | 32-37 | 34 |
| with viscosity | | | |
| midpoint 1.7 dl/g) | | | |
| HPMCAS | 60-70 | 62-66 | 64 |
| P407 | 0.5-5 | 1-3 | 2 |

| Time-dependent | | | |
|---|---|---|---|
| disintegrating | | | |
| matrix | Formulation 1 | Formulation 2 | Formulation 3 |
| PCL (viscosity | 40-50 | 43-47 | 44.95 |
| midpoint 1.2 dl/g) | | | |
| PDLG5004A | 30-40 | 33-37 | 35 |
| PDLG5004 | 10-25 | 15-20 | 18 |
| PEO(100k) | 0.5-5 | 1-3 | 2 |
| coloring (optional) | 0.005-0.2 | 0.01-0.1 | 0.05 (e.g. E172) |

| Release rate- | | | |
|---|---|---|---|
| modulating film | Formulation 1 | Formulation 2 | Formulation 3 |
| PCL (such as PC17 | 67-77 | 71-76 | 73.5 |
| with viscosity | | | |
| midpoint 1.7 dl/g) | | | |
| VA64 | 20-30 | 22-27 | 24.5 |
| Mg stearate | 0.5-5 | 1-3 | 2.0 |

In one embodiment, exemplary amounts for the various components of the risperidone dosage form are provided in the tables below. The amounts are given in approximate weight percent, with the understanding that when ranges are provided, the amounts are chosen so as to add up to 100%.

In one embodiment, exemplary amounts for the various components of the risperidone dosage form are provided in the tables below. The amounts are given in approximate weight percent, with the understanding that when ranges are provided, the amounts are chosen so as to add up to 100%.

| Carrier polymer- | | | |
|---|---|---|---|
| arm segment | Formulation 1 | Formulation 2 | Formulation 3 |
| risperidone (or | 30-40 | 33-37 | 35.0 |
| pharm. accept. salt) | | | |
| PCL (such as PC17 | 51-61 | 54-58 | 55.9 |
| with viscosity | | | |
| midpoint 1.7 dl/g) | | | |
| VA64 | 2-8 | 4-6 | 5.0 |
| P407 | 1-5 | 2-4 | 3.0 |
| vitE | 0.1-1 | 0.2-0.8 | 0.5 |
| SiO₂ | 0.1-1 | 0.2-0.8 | 0.5 |
| coloring | 0.01-0.5 | 0.05-0.2 | 0.1 (e.g. 0.05 |
| (optional) | | | blue, 0.05 yellow) |

| Inactive spacer | Formulation 1 | Formulation 2 | Formulation 3 |
|---|---|---|---|
| PCL (such as PC17 | 61-71 | 64-69 | 66.45 |
| with viscosity | | | |
| midpoint 1.7 dl/g) | | | |
| VA64 | 27-37 | 30-34 | 32 |
| P407 | 0.2-4 | 0.5-2.5 | 1.5 |
| coloring (optional) | 0.005-0.2 | 0.01-0.1 | 0.05 (e.g. |
| | | | Blue #1) |

| Enteric | | | |
|---|---|---|---|
| disintegrating | | | |
| matrix | Formulation 1 | Formulation 2 | Formulation 3 |
| PCL (such as PC17 | 30-40 | 32-37 | 34 |
| with viscosity | | | |
| midpoint 1.7 dl/g) | | | |
| HPMCAS | 60-70 | 62-66 | 64 |
| P407 | 0.5-5 | 1-3 | 2 |

| Time-dependent | | | |
|---|---|---|---|
| disintegrating | | | |
| matrix | Formulation 1 | Formulation 2 | Formulation 3 |
| PCL (viscosity | 45-55 | 48-52 | 49.95 |
| midpoint 1.2 dl/g) | | | |
| PDLG5004A | 33-43 | 36-40 | 38 |
| PDLG5004 | 5-15 | 8-12 | 10 |
| PEO(100k) | 0.5-5 | 1-3 | 2 |
| coloring (optional) | 0.005-0.2 | 0.01-0.1 | 0.05 (e.g. E172) |

| Release rate- | | | |
|---|---|---|---|
| modulating film | Formulation 1 | Formulation 2 | Formulation 3 |
| PCL (such as PC17 | 67-77 | 71-76 | 73.5 |
| with viscosity | | | |
| midpoint 1.7 dl/g) | | | |
| VA64 | 20-30 | 22-27 | 24.5 |
| Mg stearate | 0.5-5 | 1-3 | 2.0 |

In one embodiment, exemplary amounts for the various components of the risperidone dosage form are provided in the tables below. The amounts are given in approximate weight percent, with the understanding that when ranges are provided, the amounts are chosen so as to add up to 100%.

| Carrier polymer- | | | |
|---|---|---|---|
| arm segment | Formulation 1 | Formulation 2 | Formulation 3 |
| risperidone (or | 30-40 | 33-37 | 35.0 |
| pharm. accept. salt) | | | |
| PCL (such as PC17 | 51-61 | 54-58 | 55.9 |
| with viscosity | | | |
| midpoint 1.7 dl/g) | | | |
| VA64 | 2-8 | 4-6 | 5.0 |
| P407 | 1-5 | 2-4 | 3.0 |
| vitE | 0.1-1 | 0.2-0.8 | 0.5 |
| SiO₂ | 0.1-1 | 0.2-0.8 | 0.5 |
| coloring (optional) | 0.01-0.5 | 0.05-0.2 | 0.1 (e.g. 0.05 |
| | | | blue, 0.05 yellow) |

| Inactive spacer | Formulation 1 | Formulation 2 | Formulation 3 |
|---|---|---|---|
| PCL (such as PC17 | 61-71 | 64-69 | 66.45 |
| with viscosity | | | |
| midpoint 1.7 dl/g) | | | |
| VA64 | 27-37 | 30-34 | 32 |
| P407 | 0.2-4 | 0.5-2.5 | 1.5 |
| coloring (optional) | 0.005-0.2 | 0.01-0.1 | 0.05 (e.g. |
| | | | Blue #1) |

| Enteric | | | |
|---|---|---|---|
| disintegrating | | | |
| matrix | Formulation 1 | Formulation 2 | Formulation 3 |
| PCL (such as PC17, | 30-40 | 32-37 | 34 |
| viscosity midpoint | | | |
| 1.7 dl/g) | | | |
| HPMCAS | 60-70 | 62-66 | 64 |
| P407 | 0.5-5 | 1-3 | 2 |

| Time-dependent | | | |
|---|---|---|---|
| disintegrating | | | |
| matrix | Formulation 1 | Formulation 2 | Formulation 3 |
| PCL (viscosity | 45-55 | 48-52 | 49.95 |
| midpoint 1.2 dl/g) | | | |
| PDLG5004A | 30-40 | 33-37 | 35 |
| PDLG5004 | 8-18 | 11-15 | 13 |
| PEO(100k) | 0.5-5 | 1-3 | 2 |
| coloring (optional) | 0.005-0.2 | 0.01-0.1 | 0.05 (e.g. E172) |

| Release rate- | | | |
|---|---|---|---|
| modulating film | Formulation 1 | Formulation 2 | Formulation 3 |
| PCL (such as PC17 | 67-77 | 71-76 | 73.5 |
| with viscosity | | | |
| midpoint 1.7 dl/g) | | | |
| VA64 | 20-30 | 22-27 | 24.5 |
| Mg stearate | 0.5-5 | 1-3 | 2.0 |

In one embodiment, exemplary amounts for the various components of the risperidone dosage form are provided in the tables below. The amounts are given in approximate weight percent, with the understanding that when ranges are provided, the amounts are chosen so as to add up to 100%.

| Carrier polymer- | | | |
|---|---|---|---|
| arm segment | Formulation 1 | Formulation 2 | Formulation 3 |
| risperidone (or | 30-40 | 33-37 | 35.0 |
| pharm. accept. salt) | | | |
| PCL (such as PC17 | 51-61 | 54-58 | 55.9 |
| with viscosity | | | |
| midpoint 1.7 dl/g) | | | |
| VA64 | 2-8 | 4-6 | 5.0 |
| P407 | 1-5 | 2-4 | 3.0 |
| vitE | 0.1-1 | 0.2-0.8 | 0.5 |
| SiO₂ | 0.1-1 | 0.2-0.8 | 0.5 |
| coloring (optional) | 0.01-0.5 | 0.05-0.2 | 0.1 (e.g. 0.05 |
| | | | blue, 0.05 yellow) |

| | Formulation 1 | Formulation 2 | Formulation 3 |
|---|---|---|---|
| Inactive spacer | | | |
| PCL (such as PC17 | 61-71 | 64-69 | 66.45 |
| with viscosity | | | |
| midpoint 1.7 dl/g) | | | |
| VA64 | 27-37 | 30-34 | 32 |
| P407 | 0.2-4 | 0.5-2.5 | 1.5 |
| coloring | 0.005-0.2 | 0.01-0.1 | 0.05 (e.g. |
| (optional) | | | Blue #1) |
| Enteric disintegrating | | | |
| matrix | | | |
| PCL (such as PC17 | 30-40 | 32-37 | 34 |
| with viscosity | | | |
| midpoint 1.7 dl/g) | | | |
| HPMCAS | 60-70 | 62-66 | 64 |
| P407 | 0.5-5 | 1-3 | 2 |

| Time-dependent | | | |
|---|---|---|---|
| disintegrating | | | |
| matrix | Formulation 1 | Formulation 2 | Formulation 3 |
| PCL (viscosity | 45-55 | 48-52 | 49.95 |
| midpoint 1.2 dl/g) | | | |
| PDLG5004A | 27-37 | 30-34 | 31.75 |
| PDLG5004 | 10-22 | 14-18 | 15.75 |
| PEO(100k) | 0.5-5 | 1.5-3.5 | 2.5 |
| coloring (optional) | 0.005-0.2 | 0.01-0.1 | 0.05 (e.g. E172) |

| Release rate- | | | |
|---|---|---|---|
| modulating film | Formulation 1 | Formulation 2 | Formulation 3 |
| PCL (such as PC17 | 67-77 | 71-76 | 73.5 |
| with viscosity | | | |
| midpoint 1.7 dl/g) | | | |
| VA64 | 20-30 | 22-27 | 24.5 |
| Mg stearate | 0.5-5 | 1-3 | 2.0 |

The assembled arms can comprise 1) a first disintegrating matrix; 2) a first inert segment; 3) a second disintegrating matrix; 4) a second inert segment; 5) the drug eluting segment, wherein the drug eluting segment comprises a carrier polymer, and risperidone or a salt thereof, and wherein the drug eluting segment further comprises a coating comprising a release rate-modulating polymer film; and 6) a third inert segment, which can be arranged in various orders. One such order is, starting from the proximal end which is attached to the central elastomer, and proceeding to the distal end: (a first disintegrating matrix) (a first inert segment) (a second disintegrating matrix) (a second inert segment) (a drug eluting segment) (a third inert segment). In some embodiments, the third inert segment is an inactive spacer. In some embodiments, the first and second inert segments are rPCL spacers. Optional rPCL spacers (inert segments) of about 0.2-2 mm length, such as about 0.5 mm length, can be inserted between any two components above, or added to the outer tip of the assembled arm, or between the inner tip of the assembled arm and the elastomeric core.

Approximate dimensions for the length of the segments on an exemplary drug-eluting arm are provided below Drug-eluting-arm

| | Dimension set | Dimension set | Dimension set |
|---|---|---|---|
| Component | 1 | 2 | 3 |
| Carrier polymer- | 4-12 mm length | 7-9 mm length | 7.8 mm length |
| agent segment | | | |
| Inactive segment | 2-8 mm length | 4-6 mm length | 5.3 mm length |
| (Third inert | | | |
| segment) | | | |
| Inert segment | 0.1-2 mm length | 0.25-1 mm length | 0.5 mm length |
| (First, second) | | | |
| Enteric disinte- | 0.5-5 mm length | 1-3 mm length | 1.85 mm length |
| grating matrix | | | |
| Timed disinte- | 0.25-5 mm length | 0.5-2 mm length | 1.0 mm length |
| grating matrix | | | |

Approximate dimensions for the length and thickness of the segments on an exemplary drug-eluting arm are provided below

### Drug-Eluting-Arm

| Component | Dimension set 1 | Dimension set 2 | Dimension set 3 |
|---|---|---|---|
| Carrier polymer- | 4-12 mm length | 7-9 mm length | 7.8 mm length |
| agent segment | 3.0-3.7 mm thickness | 3.2-3.4 mm thickness | 3.3 mm thickness |
| Inactive segment | 2-8 mm length | 4-6 mm length | 5.3 mm length |
| (Third inert segment) | 3.0-3.7 mm thickness | 3.2-3.4 mm thickness | 3.3 mm thickness |
| Inert segment (First, | 0.1-2 mm length | 0.25-1 mm length | 0.5 mm length |
| second) | 3.0-3.7 mm thickness | 3.2-3.4 mm thickness | 3.3 mm thickness |
| Enteric disintegrating | 0.5-5 mm length | 1-3 mm length | 1.85 mm length |
| matrix | 3.0-3.7 mm thickness | 3.2-3.4 mm thickness | 3.3 mm thickness |
| Timed disintegrating | 0.25-5 mm length | 0.5-2 mm length | 1.0 mm length |
| matrix | 3.0-3.7 mm thickness | 3.2-3.4 mm thickness | 3.3 mm thickness |

Approximate dimensions for the length and thickness of the segments on an exemplary drug-eluting arm are provided below:

### Drug-Eluting-Arm

| Component | Dimension set 1 | Dimension set 2 | Dimension set 3 |
|---|---|---|---|
| Carrier polymer- | 4-12 mm length | 7-9 mm length | 7.8 mm length |
| agent segment | 3.0-3.7 mm thickness | 3.2-3.4 mm thickness | 3.3 mm thickness |
| Inactive segment | 2-8 mm length | 4-6 mm length | 5.3 mm length |
| (Third inert segment) | 2.8-3.5 mm thickness | 3.0-3.2 mm thickness | 3.1 mm thickness |
| Inert segment (First, | 0.1-2 mm length | 0.25-1 mm length | 0.5 mm length |
| second) | 3.0-3.7 mm thickness | 3.2-3.4 mm thickness | 3.3 mm thickness |
| Enteric disintegrating | 0.5-5 mm length | 1-3 mm length | 1.85 mm length |
| matrix | 3.0-3.7 mm thickness | 3.2-3.4 mm thickness | 3.3 mm thickness |
| Timed disintegrating | 0.25-5 mm length | 0.5-2 mm length | 1.0 mm length |
| matrix | 3.0-3.7 mm thickness | 3.2-3.4 mm thickness | 3.3 mm thickness |

The assembled arms can comprise 1) a first disintegrating matrix; 2) a first inert segment; 3) a second disintegrating matrix; 4) a second inert segment; and 5) a third inert segment, which can be arranged in various orders. One such order is, starting from the proximal end which is attached to the central elastomer, and proceeding to the distal end: (a first disintegrating matrix) (a first inert segment) (a second disintegrating matrix) (a second inert segment) (a third inert segment). Optional rPCL spacers (inert segments) of about 0.2-2 mm length, such as about 0.5 mm length, can be inserted between any two components below, or added to the outer tip of the assembled arm, or between the inner tip of the assembled arm and the elastomeric core. In some embodiments, the third inert segment is an inactive spacer. It will be appreciated that this embodiment of the assembled arm lacks a drug-eluting segment, and can be used when it is desired to use one or more non-drug-eluting arms for the risperidone dosage form.

Approximate dimensions for the length of the segments on an exemplary non-drug-eluting arm are provided below

### Drug-Eluting-Arm

| | Dimension | Dimension | Dimension |
|---|---|---|---|
| Component | set 1 | set 2 | set 3 |
| Inactive segment | 8-16 mm | 12-14 mm | 13.1 mm |
| (Third inert segment) | length | length | length |
| Inert segment | 0.1-2 mm | 0.25-1 mm | 0.5 mm |
| (First, and second) | length | length | length |
| Enteric disintegrating | 0.5-5 mm | 1-3 mm | 1.85 mm |
| matrix | length | length | length |
| Timed disintegrating | 0.25-5 mm | 0.5-2 mm | 1.0 mm |
| matrix | length | length | length |

Approximate dimensions for the length and thickness of the segments on an exemplary non-drug-eluting arm are provided below

### Non-Drug-Eluting-Arm

| Component | Dimension set 1 | Dimension set 2 | Dimension set 3 |
|---|---|---|---|
| Inactive segment | 8-16 mm length | 12-14 mm length | 13.1 mm length |
| (Third inert segment) | 3.0-3.7 mm thickness | 3.2-3.4 mm thickness | 3.3 mm thickness |
| Inert segment (First, | 0.1-2 mm length | 0.25-1 mm length | 0.5 mm length |
| and second) | 3.0-3.7 mm thickness | 3.2-3.4 mm thickness | 3.3 mm thickness |
| Enteric disintegrating | 0.5-5 mm length | 1-3 mm length | 1.85 mm length |
| matrix | 3.0-3.7 mm thickness | 3.2-3.4 mm thickness | 3.3 mm thickness |
| Timed disintegrating | 0.25-5 mm length | 0.5-2 mm length | 1.0 mm length |
| matrix | 3.0-3.7 mm thickness | 3.2-3.4 mm thickness | 3.3 mm thickness |

Approximate dimensions for the length and thickness of the segments on an exemplary non-drug-eluting arm are provided below

### Non-Drug-Eluting-Arm

| Component | Dimension set 1 | Dimension set 2 | Dimension set 3 |
|---|---|---|---|
| Inactive segment | 8-16 mm length | 12-14 mm length | 13.1 mm length |
| (Third inert segment) | 2.8-3.5 mm thickness | 3.0-3.2 mm thickness | 3.1 mm thickness |
| Inert segment (First, | 0.1-2 mm length | 0.25-1 mm length | 0.5 mm length |
| and second) | 3.0-3.7 mm thickness | 3.2-3.4 mm thickness | 3.3 mm thickness |
| Enteric disintegrating | 0.5-5 mm length | 1-3 mm length | 1.85 mm length |
| matrix | 3.0-3.7 mm thickness | 3.2-3.4 mm thickness | 3.3 mm thickness |
| Timed disintegrating | 0.25-5 mm length | 0.5-2 mm length | 1.0 mm length |
| matrix | 3.0-3.7 mm thickness | 3.2-3.4 mm thickness | 3.3 mm thickness |

The gastric residence systems or dosage forms above, while described as being risperidone-formulated, are not limited as such, and can be used with other drugs by replacing the segment(s) containing risperidone and/or replacing inert segment(s), with segments containing other drugs.

### Exemplary Gastric Residence Systems

The following gastric residence systems are exemplary to better illustrate certain embodiments of the system described herein. As these examples are only exemplary, they are not intended to limit the gastric residence system described herein. One skilled in the art, in view of the provided disclosure, would be able to contemplate additional configurations of the gastric residence system. Any described gastric residence system shown as being risperidone-formulated, is not limited as such, and can be used with other drugs by replacing the segment(s) containing risperidone and/or replacing inert segment(s), with segments containing other drugs.

In some embodiments, the gastric residence system comprises at least one arm including a drug eluting segment, wherein the arm comprises: (a) a first inert segment as described in any of the embodiments of inert segment above, (b) a timed disintegrating matrix as described in any of the embodiments above, (c) a second inert segment as described in any of the embodiments of inert segment above, (d) an enteric disintegrating matrix as described in any of the embodiments above, (e) a third inert segment as described in any of the embodiments of inert segment above, (f) a drug eluting segment as described in any of the embodiments described above, (g) a fourth inert segment as described in any of the embodiments of inert segment above, and (h) a third disintegrating matrix as described in any of the embodiments above. The first inert segment can be attached to a central elastomer.

In some embodiments, the gastric residence system comprises at least one arm including a drug eluting segment, wherein the arm comprises: (a) a first inert segment as described in any of the embodiments of inert segment above (such as any one of IS-1, IS-2 or IS-3), (b) a timed disintegrating matrix as described in any of the embodiments above (such as any one of T-DM1, T-DM2, T-DM3, T-DM4, T-DM5, T-DM6), (c) a second inert segment as described in any of the embodiments of inert segment above (such as any one of IS-1, IS-2 or IS-3), (d) an enteric disintegrating matrix as described in any of the embodiments above (such as E-DM1 or E-DM2), (e) a third inert segment as described in any of the embodiments of inert segment above (such as any one of IS-1, IS-2 or IS-3), (f) a drug eluting segment as described in any of the embodiments described above (such as CP-1), (g) a fourth inert segment as described in any of the embodiments of inert segment above (such as any one of IS-1, IS-2 or IS-3), and (h) a third disintegrating matrix as described in any of the embodiments above (such as ODMTEP). The drug-eluting arm may comprise an optional fifth inert segment as described in any of the embodiments of inert segment above (such as any one of IS-1, IS-2 or IS-3). The described segments can be arranged in any order. One such order is, starting from the proximal end which is attached to the central elastomer, and proceeding to the distal end: (a first inert segment) (a timed disintegrating matrix) (a second inert segment) (an enteric disintegrating matrix) (a third inert segment) (a fourth inert segment) (a drug eluting segment) (a third disintegrating matrix segment). Another such order is, starting from the proximal end which is attached to the central elastomer, and proceeding to the distal end: (a first inert segment) (a timed disintegrating matrix) (a second inert segment) (an enteric disintegrating matrix) (a third inert segment) (a fourth inert segment) (a drug eluting segment) (a fifth inert segment) (a third disintegrating matrix segment). The first inert segment can be attached to a central elastomer.

In some embodiments, the gastric residence system comprises at least one arm including a drug eluting segment, wherein the arm can be attached to a central elastomer, and the arm comprises one or more of: (a) a first inert segment, (b) a timed disintegrating matrix, (c) a second inert segment, (d) an enteric disintegrating matrix, (e) a third inert segment, (f) a drug eluting segment, (g) a fourth inert segment, and (h) a third disintegrating matrix, wherein:
the central elastomer comprises liquid silicone rubber (LSR) having a hardness of about 40 to about 65 durometer;
(a) the first inert segment comprises about 65 wt % to about 75 wt % PCL, and about 25 wt % to about 35 wt % (BiO)₂CO₃;
(b) the timed disintegrating matrix comprises about 40 wt % to about 50 wt % PCL, about 30 wt % to about 40 wt % of acid terminated copolymer of DL-lactide and glycolide (50/50 molar ratio) having a viscosity midpoint of about 0.4 dl/g, about 10 wt % to about 25 wt % of copolymer of DL-lactide and glycolide (50/50 molar ratio) having a viscosity midpoint of about 0.4 dl/g, about 0.5 wt % to about 5 wt % of polyethylene glycol 100 k, and about 0.005 wt % to about 0.2 wt % color-absorbing dye E172;
(c) the second inert segment comprises about 65 wt % to about 75 wt % PCL, and about 25 wt % to about 35 wt % (BiO)₂CO₃;
(d) the enteric disintegrating matrix comprises about 59 wt % to about 69 wt % HPMCAS, about 29 wt % to about 39 wt % PCL, and about 0.5 wt % to about 5 wt % poloxamer (such as P407) and optionally about 0.01 wt % to about 0.2 wt % iron oxide (such as E172); (e) the third inert segment comprises about 65 wt % to about 75 wt % PCL, and about 25 wt % to about 35 wt % (BiO)₂CO₃;
(f) the drug-eluting segment comprises about 30 wt % to about 40 wt % of risperidone, about 51 wt % to about 61 wt % of PCL, about 2 wt % to about 8 wt % of VA64, about 1 wt % to about 5 wt % of P407, about 0.1 wt % to about 1 wt % of Vitamin E succinate, about 0.1 wt % to about 1 wt % of SiO₂, and about 0.01 wt % to about 0.5 wt % of pigment;
(g) the fourth inert segment comprises about 61 wt % to about 71 wt % PCL, about 27 wt % to about 37 wt % copovidone, about 0.2 wt % to about 4 wt % poloxamer and optionally about 0.005 wt % to about 0.2 wt % color-absorbing dye FD&C Blue #1; and/or
(h) the third disintegrating matrix comprises about 60 wt % to about 70 wt % HPMCAS, about 25 wt % to about 35 wt % PCL, about 1 wt % to about 5 wt % propylene glycol and about 1 wt % to about 5 wt % stearic acid and optionally about 0.01 wt % to about 0.5 wt % iron oxide.

In some embodiments, the gastric residence system comprises at least one arm including a drug eluting segment, wherein the arm can be attached to a central elastomer, and the arm comprises one or more of: (a) a first inert segment, (b) a timed disintegrating matrix, (c) a second inert segment, (d) an enteric disintegrating matrix, (e) a third inert segment, (f) a drug eluting segment, (g) a fourth inert segment, and (h) a third disintegrating matrix, wherein:
the central elastomer comprises liquid silicone rubber (LSR) having a hardness of about 45 to about 55 durometer;
(a) the first inert segment comprises about 68 wt % to about 72 wt % PCL, and about 28 wt % to about 32 wt % (BiO)₂CO₃;
(b) the time-dependent disintegrating matrix comprises about 43 wt % to about 47 wt % PCL, about 33 wt % to about 37 wt % of acid terminated copolymer of DL-lactide and glycolide (50/50 molar ratio) having a viscosity midpoint of about 0.4 dl/g, about 15 wt % to about 20 wt % of copolymer of DL-lactide and glycolide (50/50 molar ratio) having a viscosity midpoint of about 0.4 dl/g, about 1 wt % to about 3 wt % of polyethylene glycol 100 k, and about 0.01 wt % to about 0.1 wt % color-absorbing dye E172;
(c) the second inert segment comprises about 68 wt % to about 72 wt % PCL, and about 28 wt % to about 32 wt % (BiO)₂CO₃;
(d) the enteric disintegrating matrix comprises about 62 wt % to about 66 wt % HPMCAS, about 32 wt % to about 36 wt % PCL, and about 1 wt % to about 3 wt % poloxamer (such as P407) and optionally about 0.05 wt % to about 0.15 wt % iron oxide (such as E172);
(e) the third inert segment comprises about 68 wt % to about 72 wt % PCL, and about 28 wt % to about 32 wt % (BiO)₂CO₃;
(f) the drug-eluting segment comprises about 33 wt % to about 37 wt % of risperidone, about 54 wt % to about 58 wt % of PCL, about 4 wt % to about 6 wt % of VA64, about 2 wt % to about 4 wt % of P407, about 0.2 wt % to about 0.8 wt % of Vitamin E succinate, about 0.2 wt % to about 0.8 wt % of SiO₂, and about 0.05 wt % to about 0.15 wt % of pigment;
(g) the fourth inert segment comprises about 64 wt % to about 69 wt % PCL, about 30 wt % to about 34 wt % copovidone, about 0.5 wt % to about 2.5 wt % poloxamer and optionally about 0.01 wt % to about 0.1 wt % color-absorbing dye FD&C Blue #1; and/or
(h) the third disintegrating matrix comprises about 63 wt % to about 67 wt % HPMCAS, about 28 wt % to about 32 wt % PCL, about 2 wt % to about 3 wt % propylene glycol and about 2 wt % to about 3 wt % stearic acid and optionally about 0.05 wt % to about 0.15 wt % iron oxide.

In some embodiments, the gastric residence system comprises at least one arm including a drug eluting segment, wherein the arm can be attached to a central elastomer, and the arm comprises one or more of: (a) a first inert segment, (b) a timed disintegrating matrix, (c) a second inert segment, (d) an enteric disintegrating matrix, (e) a third inert segment, (f) a drug eluting segment, (g) a fourth inert segment, and (h) a third disintegrating matrix, wherein:
the central elastomer comprises liquid silicone rubber (LSR) having a hardness of about 50 durometer;
(a) the first inert segment comprises about 70 wt % PCL, and about 30 wt % (BiO)₂CO₃;
(b) time-dependent disintegrating matrix, the time-dependent disintegrating matrix comprises about 44.95 wt % PCL, about 35 wt % of acid terminated copolymer of DL-lactide and glycolide (50/50 molar ratio) having a viscosity midpoint of about 0.4 dl/g, about 18 wt % of copolymer of DL-lactide and glycolide (50/50 molar ratio) having a viscosity midpoint of about 0.4 dl/g, about 2 wt % of polyethylene glycol 100 k and about 0.005 wt % to about 0.2 wt %, such as about 0.05% color-absorbing dye E172;
(c) the second inert segment comprises about 70 wt % PCL, and about 30 wt % (BiO)₂CO₃;
(d) the enteric disintegrating matrix comprises about 63.95 wt % HPMCAS, about 33.95 wt % PCL, and about 2 wt % poloxamer (such as P407) and about 0.1 wt % iron oxide (such as E172);
(e) the third inert segment comprises about 70 wt % PCL, and about 30 wt % (BiO)₂CO₃;
(f) drug-eluting segment comprises about 35.0 wt % of risperidone, about 55.9 wt % of PCL, about 5.0 wt % of VA64, about 3.0 wt % of P407, about 0.5 wt % of Vitamin E succinate, about 0.5 wt % of SiO₂, and about 0.1 wt % of pigment;
(g) the fourth inert segment comprises about 66.45 wt % PCL, about 32 wt % copovidone, about 1.5 wt % poloxamer and optionally about 0.05 wt % color-absorbing dye FD&C Blue #1; and/or
(h) the third disintegrating matrix comprises 64.9 wt % HPMCAS, about 30 wt % PCL, about 2.5 wt % propylene glycol and about 2.5 wt % stearic acid and optionally about 0.1 wt % iron oxide, for example about 0.025% ferrosoferric oxide and about 0.075% FD&C Red 40.

In some embodiments, the gastric residence system comprises at least one arm excluding a drug eluting segment, wherein the arm can be attached to a central elastomer, and the arm comprises one or more of: (a) a first inert segment as described in any of the embodiments of inert segment above, (b) a timed disintegrating matrix as described in any of the embodiments above, (c) a second inert segment as described in any of the embodiments of inert segment above, (d) an enteric disintegrating matrix as described in any of the embodiments above, (e) a third inert segment as described in any of the embodiments of inert segment above, (f) a drug-free segment as described in any of the embodiments described above, (g) a fourth inert segment as described in any of the embodiments of inert segment above, and (h) a third disintegrating matrix as described in any of the embodiments above.

In some embodiments, the gastric residence system comprises at least one arm excluding a drug eluting segment, wherein the drug-free arm can be attached to a central elastomer, and the arm comprises one or more of: (a) a first inert segment as described in any of the embodiments of inert segment above, (b) a timed disintegrating matrix as described in any of the embodiments above, (c) a second inert segment as described in any of the embodiments of inert segment above, (d) an enteric disintegrating matrix as described in any of the embodiments above, (e) a third inert segment as described in any of the embodiments of inert segment above, (f) a fourth inert segment as described in any of the embodiments of inert segment above, and (g) a third disintegrating matrix as described in any of the embodiments above.

In some embodiments, the gastric residence system comprises at least one arm excluding a drug eluting segment, wherein the drug-free arm can be attached to a central elastomer, and the arm comprises one or more of: (a) a first inert segment as described in any of the embodiments of inert segment above (such as any one of IS-1, IS-2 or IS-3), (b) a timed disintegrating matrix as described in any of the embodiments above (such as any one of T-DM1, T-DM2, T-DM3, T-DM4, T-DM5, T-DM6), (c) a second inert segment as described in any of the embodiments of inert segment above (such as any one of IS-1, IS-2 or IS-3), (d) an enteric disintegrating matrix as described in any of the embodiments above (such as E-DM1 or E-DM2), (e) a third inert segment as described in any of the embodiments of inert segment above (such as any one of IS-1, IS-2 or IS-3), (f) a fourth inert segment as described in any of the embodiments of inert segment above (such as any one of IS-1, IS-2 or IS-3), and (g) a third disintegrating matrix as described in any of the embodiments above (such as ODMTEP). The drug-free arm may comprise an optional fifth inert segment as described in any of the embodiments of inert segment above (such as any one of IS-1, IS-2 or IS-3). The described segments can be arranged in any order. One such order is, starting from the proximal end which is attached to the central elastomer, and proceeding to the distal end: (a first inert segment) (a timed disintegrating matrix) (a second inert segment) (an enteric disintegrating matrix) (a third inert segment) (a fourth inert segment) (a third disintegrating matrix segment). Another such order is, starting from the proximal end which is attached to the central elastomer, and proceeding to the distal end: (a first inert segment) (a timed disintegrating matrix) (a second inert segment) (an enteric disintegrating matrix) (a third inert segment) (a fourth inert segment) (a fifth inert segment) (a third disintegrating matrix segment). The first inert segment can be attached to a central elastomer.

In some embodiments that can be combined with any of the embodiments herein, a filament is wrapped circumferentially around a gastric residence system (e.g. by connecting the distal ends of each arm). The filament circumferentially wrapped around a gastric residence system and connecting one or more the arms of the risperidone dosage form can be a non-disintegrating filament. In some embodiments, the filament comprises thermoplastic polyurethane. In some embodiments, the filament comprises methylene bis(4-phenylisocyanate), poly(tetramethylene oxide), and/or 1,4-butanediol.

In some embodiments, the gastric residence system comprises at least one arm excluding a drug eluting segment, wherein the arm can be attached to a central elastomer, and the arm comprises one or more of: (a) a first inert segment, (b) a timed disintegrating matrix, (c) a second inert segment, (d) an enteric disintegrating matrix, (e) a third inert segment, (f) a fourth inert segment, and (g) a third disintegrating matrix, wherein:
the central elastomer comprises liquid silicone rubber (LSR) having a hardness of about 40 to about 65 durometer;
(a) the first inert segment comprises about 65 wt % to about 75 wt % PCL, and about 25 wt % to about 35 wt % (BiO)₂CO₃;
(b) the timed disintegrating matrix comprises about 40 wt % to about 50 wt % PCL, about 30 wt % to about 40 wt % of acid terminated copolymer of DL-lactide and glycolide (50/50 molar ratio) having a viscosity midpoint of about 0.4 dl/g, about 10 wt % to about 25 wt % of copolymer of DL-lactide and glycolide (50/50 molar ratio) having a viscosity midpoint of about 0.4 dl/g, about 0.5 wt % to about 5 wt % of polyethylene glycol 100 k, and about 0.005 wt % to about 0.2 wt % color-absorbing dye E172;
(c) the second inert segment comprises about 65 wt % to about 75 wt % PCL, and about 25 wt % to about 35 wt % (BiO)₂CO₃;
(d) the enteric disintegrating matrix comprises about 59 wt % to about 69 wt % HPMCAS, about 29 wt % to about 39 wt % PCL, and about 0.5 wt % to about 5 wt % poloxamer (such as P407) and optionally about 0.01 wt % to about 0.2 wt % iron oxide (such as E172);
(e) the third inert segment comprises about 65 wt % to about 75 wt % PCL, and about 25 wt % to about 35 wt % (BiO)₂CO₃;
(f) the fourth inert segment comprises about 61 wt % to about 71 wt % PCL, about 27 wt % to about 37 wt % copovidone, about 0.2 wt % to about 4 wt % poloxamer and optionally about 0.005 wt % to about 0.2 wt % color-absorbing dye FD&C Blue #1; and/or
(g) the third disintegrating matrix comprises about 60 wt % to about 70 wt % HPMCAS, about 25 wt % to about 35 wt % PCL, about 1 wt % to about 5 wt % propylene glycol and about 1 wt % to about 5 wt % stearic acid and optionally about 0.01 wt % to about 0.5 wt % iron oxide.

In some embodiments, the gastric residence system comprises at least one arm excluding a drug eluting segment, wherein the arm can be attached to a central elastomer, and the arm comprises one or more of: (a) a first inert segment, (b) a timed disintegrating matrix, (c) a second inert segment, (d) an enteric disintegrating matrix, (e) a third inert segment, (f) a fourth inert segment, and (g) a third disintegrating matrix, wherein:
the central elastomer comprises liquid silicone rubber (LSR) having a hardness of about 45 to about 55 durometer;
(a) the first inert segment comprises about 68 wt % to about 72 wt % PCL, and about 28 wt % to about 32 wt % (BiO)₂CO₃;
(b) the time-dependent disintegrating matrix comprises about 43 wt % to about 47 wt % PCL, about 33 wt % to about 37 wt % of acid terminated copolymer of DL-lactide and glycolide (50/50 molar ratio) having a viscosity midpoint of about 0.4 dl/g, about 15 wt % to about 20 wt % of copolymer of DL-lactide and glycolide (50/50 molar ratio) having a viscosity midpoint of about 0.4 dl/g, about 1 wt % to about 3 wt % of polyethylene glycol 100 k, and about 0.01 wt % to about 0.1 wt % color-absorbing dye E172;
(c) the second inert segment comprises about 68 wt % to about 72 wt % PCL, and about 28 wt % to about 32 wt % (BiO)₂CO₃;
(d) the enteric disintegrating matrix comprises about 62 wt % to about 66 wt % HPMCAS, about 32 wt % to about 36 wt % PCL, and about 1 wt % to about 3 wt % poloxamer (such as P407) and optionally about 0.05 wt % to about 0.15 wt % iron oxide (such as E172);
(e) the third inert segment comprises about 68 wt % to about 72 wt % PCL, and about 28 wt % to about 32 wt % (BiO)₂CO₃;
(f) the fourth inert segment comprises about 64 wt % to about 69 wt % PCL, about 30 wt % to about 34 wt % copovidone, about 0.5 wt % to about 2.5 wt % poloxamer and optionally about 0.01 wt % to about 0.1 wt % color-absorbing dye FD&C Blue #1; and/or
(g) the third disintegrating matrix comprises about 63 wt % to about 67 wt % HPMCAS, about 28 wt % to about 32 wt % PCL, about 2 wt % to about 3 wt % propylene glycol and about 2 wt % to about 3 wt % stearic acid and optionally about 0.05 wt % to about 0.15 wt % iron oxide.

In some embodiments, the gastric residence system comprises at least one arm excluding a drug eluting segment, wherein the arm can be attached to a central elastomer, and the arm comprises one or more of: (a) a first inert segment, (b) a timed disintegrating matrix, (c) a second inert segment, (d) an enteric disintegrating matrix, (e) a third inert segment, (f) a fourth inert segment, and (g) a third disintegrating matrix, wherein:
the central elastomer comprises liquid silicone rubber (LSR) having a hardness of about 50 durometer;
(a) the first inert segment comprises about 70 wt % PCL, and about 30 wt % (BiO)₂CO₃;
(b) time-dependent disintegrating matrix, the time-dependent disintegrating matrix comprises about 44.95 wt % PCL, about 35 wt % of acid terminated copolymer of DL-lactide and glycolide (50/50 molar ratio) having a viscosity midpoint of about 0.4 dl/g, about 18 wt % of copolymer of DL-lactide and glycolide (50/50 molar ratio) having a viscosity midpoint of about 0.4 dl/g, about 2 wt % of polyethylene glycol 100 k and about 0.05 wt % color-absorbing dye E172;
(c) the second inert segment comprises about 70 wt % PCL, and about 30 wt % (BiO)₂CO₃;
(d) the enteric disintegrating matrix comprises about 63.95 wt % HPMCAS, about 33.95 wt % PCL, and about 2 wt % poloxamer (such as P407) and about 0.1 wt % iron oxide (such as E172);
(e) the third inert segment comprises about 70 wt % PCL, and about 30 wt % (BiO)₂CO₃;
(f) the fourth inert segment comprises about 66.45 wt % PCL, about 32 wt % copovidone, about 1.5 wt % poloxamer and optionally about 0.05 wt % color-absorbing dye FD&C Blue #1; and/or
(g) the third disintegrating matrix comprises 64.9 wt % HPMCAS, about 30 wt % PCL, about 2.5 wt % propylene glycol and about 2.5 wt % stearic acid and optionally about 0.1 wt % iron oxide, for example about 0.025% ferrosoferric oxide and about 0.075% FD&C Red 40.

In some embodiments according to any of the gastric residence systems described herein, the gastric residence system comprises at least one arm including a drug eluting segment, wherein the arm further comprises a fifth optional inert segment, wherein the fifth optional inert segment comprises about 65 wt % to about 75 wt % PCL, and about 25 wt % to about 35 wt % (BiO)₂CO₃. In some embodiments, the fifth optional inert segment comprises about 68 wt % to about 72 wt % PCL, and about 28 wt % to about 32 wt % (BiO)₂CO₃. In some embodiments, the fifth optional inert segment comprises about 70 wt % PCL, and about 30 wt % (BiO)₂CO₃.

In any of the above-described embodiments, the arm can be attached to the central elastomer at the first inert segment. That is, the first inert segment is the proximal end of the arm.

The table below provides a listing of the length of each segment in the gastric residence system. Each range or value below can be considered to be “about” the range or value indicated, or exactly the range or value indicated.

| Segment | Length | |
|---|---|---|
| Drug-eluting segment | 2.4 | mm |
| Fourth inert segment | 8 | mm |
| First, second, third or fifth inert segment | 0.5 | mm |
| Enteric disintegrating matrix | 1.85 | mm |
| Time-dependent disintegrating matrix | 1.00 | mm |
| Third disintegrating matrix | 4 | mm |

For further embodiments, the table below provides a listing of the length of each segment in a drug-eluting arm in the gastric residence system. Each range or value below can be considered to be “about” the range or value indicated, or exactly the range or value indicated.

| Segment | Length | |
|---|---|---|
| Drug-eluting segment | 2.4 | mm |
| Fourth inert segment | 5.6 | mm |
| First, second, third or fifth inert segment | 0.5 | mm |
| Enteric disintegrating matrix | 1.85 | mm |
| Time-dependent disintegrating matrix | 1.00 | mm |
| Third disintegrating matrix | 4 | mm |

The gastric residence systems above, while described as being risperidone-formulated, are not limited as such, and can be used with other drugs by replacing the segment(s) containing risperidone and/or replacing inert segment(s), with segments containing other drugs.

The following gastric residence systems are exemplary to better illustrate certain embodiments of a system described herein.

In some embodiments, the gastric residence system comprises at least one arm including a drug eluting segment, wherein the arm comprises: (a) a first inert segment as described in any of the embodiments of inert segment above, (b) a timed disintegrating matrix as described in any of the embodiments above, (c) a second inert segment as described in any of the embodiments of inert segment above, (d) an enteric disintegrating matrix as described in any of the embodiments above, (e) a third inert segment as described in any of the embodiments of inert segment above, (f) a fourth inert segment as described in any of the embodiments of inert segment above, (g) a drug eluting segment as described in any of the embodiments described above, and (h) a fifth inert segment as described in any of the embodiments above. The first inert segment can be attached to a central elastomer.

In some embodiments, the gastric residence system comprises at least one arm including a drug eluting segment, wherein the arm comprises: (a) a first inert segment as described in any of the embodiments of inert segment above (such as any one of IS-1, IS-2 or IS-3), (b) a timed disintegrating matrix as described in any of the embodiments above (such as any one of T-DM1, T-DM2, T-DM3, T-DM4, T-DM5, T-DM6), (c) a second inert segment as described in any of the embodiments of inert segment above (such as any one of IS-1, IS-2 or IS-3), (d) an enteric disintegrating matrix as described in any of the embodiments above (such as E-DM1 or E-DM2), (e) a third inert segment as described in any of the embodiments of inert segment above (such as any one of IS-1, IS-2 or IS-3), (f) a fourth inert segment as described in any of the embodiments of inert segment above (such as any one of IS-1, IS-2 or IS-3), (g) a drug eluting segment as described in any of the embodiments described above, and (h) a fifth inert segment as described in any of the embodiments above (such as any one of IS-1, IS-2 or IS-3). The drug-eluting arm may comprise an optional sixth inert segment as described in any of the embodiments of inert segment above (such as any one of IS-1, IS-2 or IS-3). The segments described may be arranged in various orders. One such order is, starting from the proximal end which is attached to the central elastomer, and proceeding to the distal end: (a first inert segment) (a timed disintegrating matrix) (a second inert segment) (an enteric disintegrating matrix) (a third inert segment) (a fourth inert segment) (a drug eluting segment) (a fifth inert segment). One such order is, starting from the proximal end which is attached to the central elastomer, and proceeding to the distal end: (a first inert segment) (a timed disintegrating matrix) (a second inert segment) (an enteric disintegrating matrix) (a third inert segment) (a fourth inert segment) (a drug eluting segment) (an optional sixth inert segment) (a fifth inert segment). The first inert segment can be attached to a central elastomer.

In some embodiments, the gastric residence system comprises at least one arm including a drug eluting segment, wherein the arm can be attached to a central elastomer, and the arm comprises one or more of: (a) a first inert segment, (b) a timed disintegrating matrix, (c) a second inert segment, (d) an enteric disintegrating matrix, (e) a third inert segment, (f) a fourth inert segment, (g) a drug eluting segment, and (h) a fifth inert segment, wherein:
the central elastomer comprises liquid silicone rubber (LSR) having a hardness of about 40 to about 65 durometer;
(a) the first inert segment comprises about 65 wt % to about 75 wt % PCL, and about 25 wt % to about 35 wt % (BiO)₂CO₃;
(b) the timed disintegrating matrix comprises about 40 wt % to about 50 wt % PCL, about 30 wt % to about 40 wt % of acid terminated copolymer of DL-lactide and glycolide (50/50 molar ratio) having a viscosity midpoint of about 0.4 dl/g, about 10 wt % to about 25 wt % of ester terminated copolymer of DL-lactide and glycolide (50/50 molar ratio) having a viscosity midpoint of about 0.4 dl/g, about 0.5 wt % to about 5 wt % of polyethylene glycol 100 k, and about 0.005 wt % to about 0.2 wt % color-absorbing dye E172;
(c) the second inert segment comprises about 65 wt % to about 75 wt % PCL, and about 25 wt % to about 35 wt % (BiO)₂CO₃;
(d) the enteric disintegrating matrix comprises about 59 wt % to about 69 wt % HPMCAS, about 29 wt % to about 39 wt % PCL, and about 0.5 wt % to about 5 wt % poloxamer (such as P407) and optionally about 0.01 wt % to about 0.2 wt % iron oxide (such as E172);
(e) the third inert segment comprises about 65 wt % to about 75 wt % PCL, and about 25 wt % to about 35 wt % (BiO)₂CO₃;
(f) the fourth inert segment comprises about 61 wt % to about 71 wt % PCL, about 27 wt % to about 37 wt % copovidone, about 0.2 wt % to about 4 wt % poloxamer and optionally about 0.005 wt % to about 0.2 wt % color-absorbing dye FD&C Blue #1;
(g) the drug-eluting segment comprises about 30 wt % to about 40 wt % of risperidone, about 51 wt % to about 61 wt % of PCL, about 2 wt % to about 8 wt % of VA64, about 1 wt % to about 5 wt % of P407, about 0.1 wt % to about 1 wt % of Vitamin E succinate, about 0.1 wt % to about 1 wt % of SiO₂, and about 0.01 wt % to about 0.5 wt % of pigment; and/or
(h) the fifth inert segment comprises about 35 wt % to about 45 wt % PCL, about 37 wt % to about 47 wt % copovidone, about 10 wt % to about 20 wt % of polyethylene glycol, about 1 wt % to about 5 wt % poloxamer and optionally about 0.0005 wt % to about 0.02 wt % color-absorbing dye E172.

In some embodiments, the gastric residence system comprises at least one arm including a drug eluting segment, wherein the arm can be attached to a central elastomer, and the arm comprises one or more of: (a) a first inert segment, (b) a timed disintegrating matrix, (c) a second inert segment, (d) an enteric disintegrating matrix, (e) a third inert segment, (f) a fourth inert segment, (g) a drug eluting segment, and (h) a fifth inert segment, wherein:
the central elastomer comprises liquid silicone rubber (LSR) having a hardness of about 45 to about 55 durometer;
(a) the first inert segment comprises about 68 wt % to about 72 wt % PCL, and about 28 wt % to about 32 wt % (BiO)₂CO₃;
(b) the time-dependent disintegrating matrix comprises about 43 wt % to about 47 wt % PCL, about 33 wt % to about 37 wt % of acid terminated copolymer of DL-lactide and glycolide (50/50 molar ratio) having a viscosity midpoint of about 0.4 dl/g, about 15 wt % to about 20 wt % of ester terminated copolymer of DL-lactide and glycolide (50/50 molar ratio) having a viscosity midpoint of about 0.4 dl/g, about 1 wt % to about 3 wt % of polyethylene glycol 100 k, and about 0.01 wt % to about 0.1 wt % color-absorbing dye E172;
(c) the second inert segment comprises about 68 wt % to about 72 wt % PCL, and about 28 wt % to about 32 wt % (BiO)₂CO₃;
(d) the enteric disintegrating matrix comprises about 62 wt % to about 66 wt % HPMCAS, about 32 wt % to about 36 wt % PCL, and about 1 wt % to about 3 wt % poloxamer (such as P407) and optionally about 0.05 wt % to about 0.15 wt % iron oxide (such as E172);
(e) the third inert segment comprises about 68 wt % to about 72 wt % PCL, and about 28 wt % to about 32 wt % (BiO)₂CO₃;
(f) the fourth inert segment comprises about 64 wt % to about 69 wt % PCL, about 30 wt % to about 34 wt % copovidone, about 0.5 wt % to about 2.5 wt % poloxamer and optionally about 0.01 wt % to about 0.1 wt % color-absorbing dye FD&C Blue #1;
(g) the drug-eluting segment comprises about 33 wt % to about 37 wt % of risperidone, about 54 wt % to about 58 wt % of PCL, about 4 wt % to about 6 wt % of VA64, about 2 wt % to about 4 wt % of P407, about 0.2 wt % to about 0.8 wt % of Vitamin E succinate, about 0.2 wt % to about 0.8 wt % of SiO₂, and about 0.05 wt % to about 0.15 wt % of pigment; and/or
(h) the fifth inert segment comprises about 38 wt % to about 42 wt % PCL, about 40 wt % to about 44 wt % copovidone, about 13 wt % to about 17 wt % of polyethylene glycol, about 2 wt % to about 4 wt % poloxamer and optionally about 0.001 wt % to about 0.01 wt % color-absorbing dye E172.

In some embodiments, the gastric residence system comprises at least one arm including a drug eluting segment, wherein the arm can be attached to a central elastomer, and the arm comprises one or more of: (a) a first inert segment, (b) a timed disintegrating matrix, (c) a second inert segment, (d) an enteric disintegrating matrix, (e) a third inert segment, (f) a fourth inert segment, (g) a drug eluting segment, and (h) a fifth inert segment, wherein:
the central elastomer comprises liquid silicone rubber (LSR) having a hardness of about 50 durometer;
(a) the first inert segment comprises about 70 wt % PCL, and about 30 wt % (BiO)₂CO₃;
(b) time-dependent disintegrating matrix, the time-dependent disintegrating matrix comprises about 44.95 wt % PCL, about 35 wt % of acid terminated copolymer of DL-lactide and glycolide (50/50 molar ratio) having a viscosity midpoint of about 0.4 dl/g, about 18 wt % of ester terminated copolymer of DL-lactide and glycolide (50/50 molar ratio) having a viscosity midpoint of about 0.4 dl/g, about 2 wt % of polyethylene glycol 100 k and about 0.005 wt % to about 0.2 wt %, such as about 0.05% color-absorbing dye E172;
(c) the second inert segment comprises about 70 wt % PCL, and about 30 wt % (BiO)₂CO₃;
(d) the enteric disintegrating matrix comprises about 63.95 wt % HPMCAS, about 33.95 wt % PCL, and about 2 wt % poloxamer (such as P407) and about 0.1 wt % iron oxide (such as E172);
(e) the third inert segment comprises about 70 wt % PCL, and about 30 wt % (BiO)₂CO₃;
(f) the fourth inert segment comprises about 66.45 wt % PCL, about 32 wt % copovidone, about 1.5 wt % poloxamer and optionally about 0.05 wt % color-absorbing dye FD&C Blue #1;
(g) drug-eluting segment comprises about 35.0 wt % of risperidone, about 55.9 wt % of PCL, about 5.0 wt % of VA64, about 3.0 wt % of P407, about 0.5 wt % of Vitamin E succinate, about 0.5 wt % of SiO₂, and about 0.1 wt % of pigment; and/or
(h) the fifth inert segment comprises about 39.995 wt % PCL, about 42 wt % copovidone, about 15 wt % of polyethylene glycol, about 3 wt % poloxamer and optionally about 0.05 wt % color-absorbing dye E172.

In some embodiments, the gastric residence system comprises at least one arm excluding a drug eluting segment, wherein the arm can be attached to a central elastomer, and the arm comprises one or more of: (a) a first inert segment as described in any of the embodiments of inert segment above, (b) a timed disintegrating matrix as described in any of the embodiments above, (c) a second inert segment as described in any of the embodiments of inert segment above, (d) an enteric disintegrating matrix as described in any of the embodiments above, (e) a third inert segment as described in any of the embodiments of inert segment above, (f) a fourth inert segment as described in any of the embodiments of inert segment above, and (g) a fifth inert segment as described in any of the embodiments above.

In some embodiments, the gastric residence system comprises at least one arm excluding a drug eluting segment, wherein the arm comprises: (a) a first inert segment as described in any of the embodiments of inert segment above (such as any one of IS-1, IS-2 or IS-3), (b) a timed disintegrating matrix as described in any of the embodiments above (such as any one of T-DM1, T-DM2, T-DM3, T-DM4, T-DM5, T-DM6), (c) a second inert segment as described in any of the embodiments of inert segment above (such as any one of IS-1, IS-2 or IS-3), (d) an enteric disintegrating matrix as described in any of the embodiments above (such as E-DM1 or E-DM2), (e) a third inert segment as described in any of the embodiments of inert segment above (such as any one of IS-1, IS-2 or IS-3), (f) a fourth inert segment as described in any of the embodiments of inert segment above (such as any one of IS-1, IS-2 or IS-3), and (g) a fifth inert segment as described in any of the embodiments above (such as any one of IS-1, IS-2 or IS-3). The drug-free arm may comprise an optional sixth inert segment as described in any of the embodiments of inert segment above (such as any one of IS-1, IS-2 or IS-3). The segments described can be arranged in various orders. One such order is, starting from the proximal end which is attached to the central elastomer, and proceeding to the distal end: (a first inert segment) (a timed disintegrating matrix) (a second inert segment) (an enteric disintegrating matrix) (a third inert segment) (a fourth inert segment) (a fifth inert segment). One such order is, starting from the proximal end which is attached to the central elastomer, and proceeding to the distal end: (a first inert segment) (a timed disintegrating matrix) (a second inert segment) (an enteric disintegrating matrix) (a third inert segment) (a fourth inert segment) (an optional sixth inert segment) (a fifth inert segment). The first inert segment can be attached to a central elastomer.

In some embodiments, the gastric residence system comprises at least one arm excluding a drug eluting segment, wherein the arm can be attached to a central elastomer, and the arm comprises one or more of: (a) a first inert segment, (b) a timed disintegrating matrix, (c) a second inert segment, (d) an enteric disintegrating matrix, (e) a third inert segment, (f) a fourth inert segment, and (g) a fifth inert segment, wherein:
the central elastomer comprises liquid silicone rubber (LSR) having a hardness of about 40 to about 65 durometer;
(a) the first inert segment comprises about 65 wt % to about 75 wt % PCL, and about 25 wt % to about 35 wt % (BiO)₂CO₃;
(b) the timed disintegrating matrix comprises about 40 wt % to about 50 wt % PCL, about 30 wt % to about 40 wt % of acid terminated copolymer of DL-lactide and glycolide (50/50 molar ratio) having a viscosity midpoint of about 0.4 dl/g, about 10 wt % to about 25 wt % of ester terminated copolymer of DL-lactide and glycolide (50/50 molar ratio) having a viscosity midpoint of about 0.4 dl/g, about 0.5 wt % to about 5 wt % of polyethylene glycol 100 k, and about 0.005 wt % to about 0.2 wt % color-absorbing dye E172;
(c) the second inert segment comprises about 65 wt % to about 75 wt % PCL, and about 25 wt % to about 35 wt % (BiO)₂CO₃;
(d) the enteric disintegrating matrix comprises about 59 wt % to about 69 wt % HPMCAS, about 29 wt % to about 39 wt % PCL, and about 0.5 wt % to about 5 wt % poloxamer (such as P407) and optionally about 0.01 wt % to about 0.2 wt % iron oxide (such as E172);
(e) the third inert segment comprises about 65 wt % to about 75 wt % PCL, and about 25 wt % to about 35 wt % (BiO)₂CO₃;
(f) the fourth inert segment comprises about 61 wt % to about 71 wt % PCL, about 27 wt % to about 37 wt % copovidone, about 0.2 wt % to about 4 wt % poloxamer and optionally about 0.005 wt % to about 0.2 wt % color-absorbing dye FD&C Blue #1; and/or
(g) the fifth inert segment comprises about 35 wt % to about 45 wt % PCL, about 37 wt % to about 47 wt % copovidone, about 10 wt % to about 20 wt % of polyethylene glycol, about 1 wt % to about 5 wt % poloxamer and optionally about 0.0005 wt % to about 0.02 wt % color-absorbing dye E172.

In some embodiments, the gastric residence system comprises at least one arm excluding a drug eluting segment, wherein the arm can be attached to a central elastomer, and the arm comprises one or more of: (a) a first inert segment, (b) a timed disintegrating matrix, (c) a second inert segment, (d) an enteric disintegrating matrix, (e) a third inert segment, (f) a fourth inert segment, and (g) a fifth inert segment, wherein:
the central elastomer comprises liquid silicone rubber (LSR) having a hardness of about 45 to about 55 durometer;
(a) the first inert segment comprises about 68 wt % to about 72 wt % PCL, and about 28 wt % to about 32 wt % (BiO)₂CO₃;
(b) the time-dependent disintegrating matrix comprises about 43 wt % to about 47 wt % PCL, about 33 wt % to about 37 wt % of acid terminated copolymer of DL-lactide and glycolide (50/50 molar ratio) having a viscosity midpoint of about 0.4 dl/g, about 15 wt % to about 20 wt % of ester terminated copolymer of DL-lactide and glycolide (50/50 molar ratio) having a viscosity midpoint of about 0.4 dl/g, about 1 wt % to about 3 wt % of polyethylene glycol 100 k, and about 0.01 wt % to about 0.1 wt % color-absorbing dye E172;
(c) the second inert segment comprises about 68 wt % to about 72 wt % PCL, and about 28 wt % to about 32 wt % (BiO)₂CO₃;
(d) the enteric disintegrating matrix comprises about 62 wt % to about 66 wt % HPMCAS, about 32 wt % to about 36 wt % PCL, and about 1 wt % to about 3 wt % poloxamer (such as P407) and optionally about 0.05 wt % to about 0.15 wt % iron oxide (such as E172);
(e) the third inert segment comprises about 68 wt % to about 72 wt % PCL, and about 28 wt % to about 32 wt % (BiO)₂CO₃;
(f) the fourth inert segment comprises about 64 wt % to about 69 wt % PCL, about 30 wt % to about 34 wt % copovidone, about 0.5 wt % to about 2.5 wt % poloxamer and optionally about 0.01 wt % to about 0.1 wt % color-absorbing dye FD&C Blue #1; and/or
(g) the fifth inert segment comprises about 38 wt % to about 42 wt % PCL, about 40 wt % to about 44 wt % copovidone, about 13 wt % to about 17 wt % of polyethylene glycol, about 2 wt % to about 4 wt % poloxamer and optionally about 0.001 wt % to about 0.01 wt % color-absorbing dye E172.

In some embodiments, the gastric residence system comprises at least one arm excluding a drug eluting segment, wherein the arm can be attached to a central elastomer, and the arm comprises one or more of: (a) a first inert segment, (b) a timed disintegrating matrix, (c) a second inert segment, (d) an enteric disintegrating matrix, (e) a third inert segment, (f) a fourth inert segment, and (g) a fifth inert segment, wherein:
the central elastomer comprises liquid silicone rubber (LSR) having a hardness of about 50 durometer;
(a) the first inert segment comprises about 70 wt % PCL, and about 30 wt % (BiO)₂CO₃;
(b) time-dependent disintegrating matrix, the time-dependent disintegrating matrix comprises about 44.95 wt % PCL, about 35 wt % of acid terminated copolymer of DL-lactide and glycolide (50/50 molar ratio) having a viscosity midpoint of about 0.4 dl/g, about 18 wt % of ester terminated copolymer of DL-lactide and glycolide (50/50 molar ratio) having a viscosity midpoint of about 0.4 dl/g, about 2 wt % of polyethylene glycol 100 k and about 0.05 wt % color-absorbing dye E172;
(c) the second inert segment comprises about 70 wt % PCL, and about 30 wt % (BiO)₂CO₃;
(d) the enteric disintegrating matrix comprises about 63.95 wt % HPMCAS, about 33.95 wt % PCL, and about 2 wt % poloxamer (such as P407) and about 0.1 wt % iron oxide (such as E172);
(e) the third inert segment comprises about 70 wt % PCL, and about 30 wt % (BiO)₂CO₃;
(f) the fourth inert segment comprises about 66.45 wt % PCL, about 32 wt % copovidone, about 1.5 wt % poloxamer and optionally about 0.05 wt % color-absorbing dye FD&C Blue #1; and/or
(g) the fifth inert segment comprises about 39.995 wt % PCL, about 42 wt % copovidone, about 15 wt % of polyethylene glycol, about 3 wt % poloxamer and optionally about 0.05 wt % color-absorbing dye E172

In some embodiments according to any of the gastric residence systems described herein, the gastric residence system comprises at least one arm including a drug eluting segment, wherein the arm further comprises a fifth optional inert segment, wherein the fifth optional inert segment comprises about 65 wt % to about 75 wt % PCL, and about 25 wt % to about 35 wt % (BiO)₂CO₃. In some embodiments, the fifth optional inert segment comprises about 68 wt % to about 72 wt % PCL, and about 28 wt % to about 32 wt % (BiO)₂CO₃. In some embodiments, the fifth optional inert segment comprises about 70 wt % PCL, and about 30 wt % (BiO)₂CO₃.

In any of the above-described embodiments, the arm can be attached to the central elastomer at the first inert segment. That is, the first inert segment is the proximal end of the arm.

The table below provides a listing of the length of each segment in a drug-eluting arm in the gastric residence system. Each range or value below can be considered to be “about” the range or value indicated, or exactly the range or value indicated.

| Segment | Length | |
|---|---|---|
| Drug-eluting segment | 2.4 | mm |
| Fourth inert segment | 5.6 | mm |
| First, second, third or fifth inert segment | 0.5 | mm |
| Enteric disintegrating matrix | 1.85 | mm |
| Time-dependent disintegrating matrix | 1.00 | mm |
| Fifth inert segment | 4 | mm |

The table below provides a listing of the length of each segment in a drug-free arm in the gastric residence system. Each range or value below can be considered to be “about” the range or value indicated, or exactly the range or value indicated.

| Segment | Length | |
|---|---|---|
| Fourth inert segment | 8 | mm |
| First, second, third or fifth inert segment | 0.5 | mm |
| Enteric disintegrating matrix | 1.85 | mm |
| Time-dependent disintegrating matrix | 1.00 | mm |
| Fifth inert segment | 4 | mm |

The gastric residence systems above, while described as being risperidone-formulated, are not limited as such, and can be used with other drugs by replacing the segment(s) containing risperidone and/or replacing inert segment(s), with segments containing other drugs.

The following gastric residence systems are exemplary to better illustrate embodiments of a system described herein.

In some embodiments, the gastric residence system comprises at least one arm including a drug eluting segment, wherein the arm comprises: (a) a timed disintegrating matrix as described in any of the embodiments above, (b) a first inert segment as described in any of the embodiments of inert segment above, (c) an enteric disintegrating matrix as described in any of the embodiments above, (d) a second inert segment as described in any of the embodiments of inert segment above, (e) a drug eluting segment as described in any of the embodiments described above, and (f) a third inert segment as described in any of the embodiments of inert segment above. The timed disintegrating matrix can be attached to a central elastomer.

In some embodiments, the gastric residence system comprises at least one arm including a drug eluting segment, wherein the arm comprises: (a) a timed disintegrating matrix as described in any of the embodiments above (such as any one of T-DM1, T-DM2, T-DM3, T-DM4, T-DM5, T-DM6), (b) a first inert segment as described in any of the embodiments of inert segment above (such as any one of IS-1, IS-2 or IS-3), (c) an enteric disintegrating matrix as described in any of the embodiments above (such as E-DM1 or E-DM2), (d) a second inert segment as described in any of the embodiments of inert segment above (such as any one of IS-1, IS-2 or IS-3), (e) a drug eluting segment as described in any of the embodiments described above (such as CP-1), and (f) a third inert segment as described in any of the embodiments of inert segment above (such as any one of IS-1, IS-2 or IS-3). The drug-eluting arm may comprise an optional fourth inert segment as described in any of the embodiments of inert segment above (such as any one of IS-1, IS-2 or IS-3). The described segments can be arranged in any order. One such order is, starting from the proximal end which is attached to the central elastomer, and proceeding to the distal end: (a timed disintegrating matrix) (a first inert segment) (an enteric disintegrating matrix) (a second inert segment) (a drug eluting segment) (a third inert segment). Another such order is, starting from the proximal end which is attached to the central elastomer, and proceeding to the distal end: (a timed disintegrating matrix) (a first inert segment) (an enteric disintegrating matrix) (a second inert segment) (a drug eluting segment) (an optional fourth inert segment) (a third inert segment). The timed disintegrating matrix can be attached to a central elastomer.

In some embodiments, a filament is wrapped circumferentially around a gastric residence system (e.g. by connecting the distal ends of each arm). The filament circumferentially wrapped around a gastric residence system and connecting one or more the arms of the risperidone dosage form can be a disintegrating filament. In some embodiments, the filament comprises poly (lactic-co-glycolic acid) and/or polyglycolic acid.

In some embodiments, the gastric residence system comprises at least one arm including a drug eluting segment, wherein the arm can be attached to a central elastomer, and the arm comprises one or more of: (a) a timed disintegrating matrix, (b) a first inert segment, (c) an enteric disintegrating matrix, (d) a second inert segment, (e) a drug eluting segment, and (f) a third inert segment, wherein:
the central elastomer comprises liquid silicone rubber (LSR) having a hardness of about 40 to about 65 durometer;
(a) the timed disintegrating matrix comprises about 40 wt % to about 50 wt % PCL, about 30 wt % to about 40 wt % of acid terminated copolymer of DL-lactide and glycolide (50/50 molar ratio) having a viscosity midpoint of about 0.4 dl/g, about 10 wt % to about 25 wt % of ester terminated copolymer of DL-lactide and glycolide (50/50 molar ratio) having a viscosity midpoint of about 0.4 dl/g, about 0.5 wt % to about 5 wt % of polyethylene glycol 100 k, and about 0.005 wt % to about 0.2 wt % color-absorbing dye E172;
(b) the first inert segment comprises about 65 wt % to about 75 wt % PCL, and about 25 wt % to about 35 wt % (BiO)₂CO₃;
(c) the enteric disintegrating matrix comprises about 59 wt % to about 69 wt % HPMCAS, about 29 wt % to about 39 wt % PCL, and about 0.5 wt % to about 5 wt % poloxamer (such as P407);
(d) the second inert segment comprises about 65 wt % to about 75 wt % PCL, and about 25 wt % to about 35 wt % (BiO)₂CO₃;
(e) the drug-eluting segment comprises about 30 wt % to about 40 wt % of risperidone, about 51 wt % to about 61 wt % of PCL, about 2 wt % to about 8 wt % of VA64, about 1 wt % to about 5 wt % of P407, about 0.1 wt % to about 1 wt % of Vitamin E succinate, about 0.1 wt % to about 1 wt % of SiO₂, and about 0.01 wt % to about 0.5 wt % of pigment; and/or
(f) the third inert segment comprises about 61 wt % to about 71 wt % PCL, about 27 wt % to about 37 wt % copovidone, about 0.2 wt % to about 4 wt % poloxamer and optionally about 0.005 wt % to about 0.2 wt % color-absorbing dye FD&C Blue #1.

In some embodiments, the gastric residence system comprises at least one arm including a drug eluting segment, wherein the arm can be attached to a central elastomer, and the arm comprises one or more of: (a) a timed disintegrating matrix, (b) a first inert segment, (c) an enteric disintegrating matrix, (d) a second inert segment, (e) a drug eluting segment, and (f) a third inert segment, wherein:
the central elastomer comprises liquid silicone rubber (LSR) having a hardness of about 40 to about 65 durometer;
(a) the timed disintegrating matrix comprises about 45 wt % to about 55 wt % PCL, about 27 wt % to about 37 wt % of acid terminated copolymer of DL-lactide and glycolide (50/50 molar ratio) having a viscosity midpoint of about 0.4 dl/g, about 10 wt % to about 22 wt % of ester terminated copolymer of DL-lactide and glycolide (50/50 molar ratio) having a viscosity midpoint of about 0.4 dl/g, about 0.5 wt % to about 5 wt % of polyethylene glycol 100 k, and about 0.005 wt % to about 0.2 wt % color-absorbing dye E172;
(b) the first inert segment comprises about 65 wt % to about 75 wt % PCL, and about 25 wt % to about 35 wt % (BiO)₂CO₃;
(c) the enteric disintegrating matrix comprises about 59 wt % to about 69 wt % HPMCAS, about 29 wt % to about 39 wt % PCL, and about 0.5 wt % to about 5 wt % poloxamer (such as P407);
(d) the second inert segment comprises about 65 wt % to about 75 wt % PCL, and about 25 wt % to about 35 wt % (BiO)₂CO₃;
(e) the drug-eluting segment comprises about 30 wt % to about 40 wt % of risperidone, about 51 wt % to about 61 wt % of PCL, about 2 wt % to about 8 wt % of VA64, about 1 wt % to about 5 wt % of P407, about 0.1 wt % to about 1 wt % of Vitamin E succinate, about 0.1 wt % to about 1 wt % of SiO₂, and about 0.01 wt % to about 0.5 wt % of pigment; and/or
(f) the third inert segment comprises about 61 wt % to about 71 wt % PCL, about 27 wt % to about 37 wt % copovidone, about 0.2 wt % to about 4 wt % poloxamer and optionally about 0.005 wt % to about 0.2 wt % color-absorbing dye FD&C Blue #1.

In some embodiments, the gastric residence system comprises at least one arm including a drug eluting segment, wherein the arm can be attached to a central elastomer, and the arm comprises one or more of: (a) a timed disintegrating matrix, (b) a first inert segment, (c) an enteric disintegrating matrix, (d) a second inert segment, (e) a drug eluting segment, and (f) a third inert segment, wherein:
the central elastomer comprises liquid silicone rubber (LSR) having a hardness of about 45 to about 55 durometer;
(a) the time-dependent disintegrating matrix comprises about 43 wt % to about 47 wt % PCL, about 33 wt % to about 37 wt % of acid terminated copolymer of DL-lactide and glycolide (50/50 molar ratio) having a viscosity midpoint of about 0.4 dl/g, about 15 wt % to about 20 wt % of ester terminated copolymer of DL-lactide and glycolide (50/50 molar ratio) having a viscosity midpoint of about 0.4 dl/g, about 1 wt % to about 3 wt % of polyethylene glycol 100 k, and about 0.01 wt % to about 0.1 wt % color-absorbing dye E172;
(b) the first inert segment comprises about 68 wt % to about 72 wt % PCL, and about 28 wt % to about 32 wt % (BiO)₂CO₃;
(c) the enteric disintegrating matrix comprises about 62 wt % to about 66 wt % HPMCAS, about 32 wt % to about 36 wt % PCL, and about 1 wt % to about 3 wt % poloxamer (such as P407);
(d) the second inert segment comprises about 68 wt % to about 72 wt % PCL, and about 28 wt % to about 32 wt % (BiO)₂CO₃;
(e) the drug-eluting segment comprises about 33 wt % to about 37 wt % of risperidone, about 54 wt % to about 58 wt % of PCL, about 4 wt % to about 6 wt % of VA64, about 2 wt % to about 4 wt % of P407, about 0.2 wt % to about 0.8 wt % of Vitamin E succinate, about 0.2 wt % to about 0.8 wt % of SiO₂, and about 0.05 wt % to about 0.15 wt % of pigment; and/or
(f) the third inert segment comprises about 64 wt % to about 69 wt % PCL, about 30 wt % to about 34 wt % copovidone, about 0.5 wt % to about 2.5 wt % poloxamer and optionally about 0.01 wt % to about 0.1 wt % color-absorbing dye FD&C Blue #1.

In some embodiments, the gastric residence system comprises at least one arm including a drug eluting segment, wherein the arm can be attached to a central elastomer, and the arm comprises one or more of: (a) a timed disintegrating matrix, (b) a first inert segment, (c) an enteric disintegrating matrix, (d) a second inert segment, (e) a drug eluting segment, and (f) a third inert segment, wherein:
the central elastomer comprises liquid silicone rubber (LSR) having a hardness of about 45 to about 55 durometer;
(a) the time-dependent disintegrating matrix comprises about 48 wt % to about 52 wt % PCL, about 30 wt % to about 34 wt % of acid terminated copolymer of DL-lactide and glycolide (50/50 molar ratio) having a viscosity midpoint of about 0.4 dl/g, about 14 wt % to about 18 wt % of ester terminated copolymer of DL-lactide and glycolide (50/50 molar ratio) having a viscosity midpoint of about 0.4 dl/g, about 1 wt % to about 3 wt % of polyethylene glycol 100 k, and about 0.01 wt % to about 0.1 wt % color-absorbing dye E172;
(b) the first inert segment comprises about 68 wt % to about 72 wt % PCL, and about 28 wt % to about 32 wt % (BiO)₂CO₃;
(c) the enteric disintegrating matrix comprises about 62 wt % to about 66 wt % HPMCAS, about 32 wt % to about 36 wt % PCL, and about 1 wt % to about 3 wt % poloxamer (such as P407);
(d) the second inert segment comprises about 68 wt % to about 72 wt % PCL, and about 28 wt % to about 32 wt % (BiO)₂CO₃;
(e) the drug-eluting segment comprises about 33 wt % to about 37 wt % of risperidone, about 54 wt % to about 58 wt % of PCL, about 4 wt % to about 6 wt % of VA64, about 2 wt % to about 4 wt % of P407, about 0.2 wt % to about 0.8 wt % of Vitamin E succinate, about 0.2 wt % to about 0.8 wt % of SiO₂, and about 0.05 wt % to about 0.15 wt % of pigment; and/or
(f) the third inert segment comprises about 64 wt % to about 69 wt % PCL, about 30 wt % to about 34 wt % copovidone, about 0.5 wt % to about 2.5 wt % poloxamer and optionally about 0.01 wt % to about 0.1 wt % color-absorbing dye FD&C Blue #1.

In some embodiments, the gastric residence system comprises at least one arm including a drug eluting segment, wherein the arm can be attached to a central elastomer, and the arm comprises one or more of: (a) a timed disintegrating matrix, (b) a first inert segment, (c) an enteric disintegrating matrix, (d) a second inert segment, (e) a drug eluting segment, and (f) a third inert segment, wherein:
the central elastomer comprises liquid silicone rubber (LSR) having a hardness of about 50 durometer;
(a) time-dependent disintegrating matrix, the time-dependent disintegrating matrix comprises about 44.95 wt % PCL, about 35 wt % of acid terminated copolymer of DL-lactide and glycolide (50/50 molar ratio) having a viscosity midpoint of about 0.4 dl/g, about 18 wt % of ester terminated copolymer of DL-lactide and glycolide (50/50 molar ratio) having a viscosity midpoint of about 0.4 dl/g, about 2 wt % of polyethylene glycol 100 k and about 0.05 wt % color-absorbing dye E172;
(b) the second inert segment comprises about 70 wt % PCL, and about 30 wt % (BiO)₂CO₃;
(c) the enteric disintegrating matrix comprises about 63.95 wt % HPMCAS, about 33.95 wt % PCL, and about 2 wt % poloxamer (such as P407);
(d) the second inert segment comprises about 70 wt % PCL, and about 30 wt % (BiO)₂CO₃;
(e) the drug-eluting segment comprises about 35.0 wt % of risperidone, about 55.9 wt % of PCL, about 5.0 wt % of VA64, about 3.0 wt % of P407, about 0.5 wt % of Vitamin E succinate, about 0.5 wt % of SiO₂, and about 0.1 wt % of pigment; and/or
(f) the third inert segment comprises about 66.45 wt % PCL, about 32 wt % copovidone, about 1.5 wt % poloxamer and optionally about 0.05 wt % color-absorbing dye FD&C Blue #1.

In some embodiments, the gastric residence system comprises at least one arm including a drug eluting segment, wherein the arm can be attached to a central elastomer, and the arm comprises one or more of: (a) a timed disintegrating matrix, (b) a first inert segment, (c) an enteric disintegrating matrix, (d) a second inert segment, (e) a drug eluting segment, and (f) a third inert segment, wherein:
the central elastomer comprises liquid silicone rubber (LSR) having a hardness of about 50 durometer;
(a) time-dependent disintegrating matrix, the time-dependent disintegrating matrix comprises about 49.95 wt % PCL, about 32 wt % of acid terminated copolymer of DL-lactide and glycolide (50/50 molar ratio) having a viscosity midpoint of about 0.4 dl/g, about 16 wt % of ester terminated copolymer of DL-lactide and glycolide (50/50 molar ratio) having a viscosity midpoint of about 0.4 dl/g, about 2 wt % of polyethylene glycol 100 k and about 0.05 wt % color-absorbing dye E172;
(b) the second inert segment comprises about 70 wt % PCL, and about 30 wt % (BiO)₂CO₃;
(c) the enteric disintegrating matrix comprises about 63.95 wt % HPMCAS, about 33.95 wt % PCL, and about 2 wt % poloxamer (such as P407);
(d) the second inert segment comprises about 70 wt % PCL, and about 30 wt % (BiO)₂CO₃;
(e) the drug-eluting segment comprises about 35.0 wt % of risperidone, about 55.9 wt % of PCL, about 5.0 wt % of VA64, about 3.0 wt % of P407, about 0.5 wt % of Vitamin E succinate, about 0.5 wt % of SiO₂, and about 0.1 wt % of pigment; and/or
(f) the third inert segment comprises about 66.45 wt % PCL, about 32 wt % copovidone, about 1.5 wt % poloxamer and optionally about 0.05 wt % color-absorbing dye FD&C Blue #1.

In some embodiments, the gastric residence system comprises at least one arm excluding a drug eluting segment, wherein the arm can be attached to a central elastomer, and the arm comprises one or more of: (a) a timed disintegrating matrix as described in any of the embodiments above, (b) a first inert segment as described in any of the embodiments of inert segment above, (c) an enteric disintegrating matrix as described in any of the embodiments above, (d) a second inert segment as described in any of the embodiments of inert segment above, and (e) a third inert segment as described in any of the embodiments of inert segment above. The timed disintegrating matrix can be attached to a central elastomer.

In some embodiments, the gastric residence system comprises at least one arm excluding a drug eluting segment, wherein the arm comprises: (a) a timed disintegrating matrix as described in any of the embodiments above (such as any one of T-DM1, T-DM2, T-DM3, T-DM4, T-DM5, T-DM6), (b) a first inert segment as described in any of the embodiments of inert segment above (such as any one of IS-1, IS-2 or IS-3), (c) an enteric disintegrating matrix as described in any of the embodiments above (such as E-DM1 or E-DM2), (d) a second inert segment as described in any of the embodiments of inert segment above (such as any one of IS-1, IS-2 or IS-3), and (e) a third inert segment as described in any of the embodiments of inert segment above (such as any one of IS-1, IS-2 or IS-3). The drug-free arm may comprise an optional fourth inert segment as described in any of the embodiments of inert segment above (such as any one of IS-1, IS-2 or IS-3). The described segments can be arranged in any order. One such order is, starting from the proximal end which is attached to the central elastomer, and proceeding to the distal end: (a timed disintegrating matrix) (a first inert segment) (an enteric disintegrating matrix) (a second inert segment) (a third inert segment). Another such order is, starting from the proximal end which is attached to the central elastomer, and proceeding to the distal end: (a timed disintegrating matrix) (a first inert segment) (an enteric disintegrating matrix) (a second inert segment) (an optional fourth inert segment) (a third inert segment). The timed disintegrating matrix can be attached to a central elastomer.

In some embodiments, a filament is wrapped circumferentially around a gastric residence system (e.g. by connecting the distal ends of each arm). The filament circumferentially wrapped around a gastric residence system and connecting one or more the arms of the risperidone dosage form can be a disintegrating filament. In some embodiments, the filament comprises poly (lactic-co-glycolic acid) and/or polyglycolic acid.

In some embodiments, the gastric residence system comprises at least one arm excluding a drug eluting segment, wherein the arm can be attached to a central elastomer, and the arm comprises one or more of: (a) a timed disintegrating matrix, (b) a first inert segment, (c) an enteric disintegrating matrix, (d) a second inert segment, (e) a drug eluting segment, and (f) a third inert segment, wherein:
the central elastomer comprises liquid silicone rubber (LSR) having a hardness of about 40 to about 65 durometer;
(a) the timed disintegrating matrix comprises about 40 wt % to about 50 wt % PCL, about 30 wt % to about 40 wt % of acid terminated copolymer of DL-lactide and glycolide (50/50 molar ratio) having a viscosity midpoint of about 0.4 dl/g, about 10 wt % to about 25 wt % of ester terminated copolymer of DL-lactide and glycolide (50/50 molar ratio) having a viscosity midpoint of about 0.4 dl/g, about 0.5 wt % to about 5 wt % of polyethylene glycol 100 k, and about 0.005 wt % to about 0.2 wt % color-absorbing dye E172;
(b) the first inert segment comprises about 65 wt % to about 75 wt % PCL, and about 25 wt % to about 35 wt % (BiO)₂CO₃;
(c) the enteric disintegrating matrix comprises about 59 wt % to about 69 wt % HPMCAS, about 29 wt % to about 39 wt % PCL, and about 0.5 wt % to about 5 wt % poloxamer (such as P407);
(d) the second inert segment comprises about 65 wt % to about 75 wt % PCL, and about 25 wt % to about 35 wt % (BiO)₂CO₃; and/or
(e) the third inert segment comprises about 61 wt % to about 71 wt % PCL, about 27 wt % to about 37 wt % copovidone, about 0.2 wt % to about 4 wt % poloxamer and optionally about 0.005 wt % to about 0.2 wt % color-absorbing dye FD&C Blue #1.

In some embodiments, the gastric residence system comprises at least one arm excluding a drug eluting segment, wherein the arm can be attached to a central elastomer, and the arm comprises one or more of: (a) a timed disintegrating matrix, (b) a first inert segment, (c) an enteric disintegrating matrix, (d) a second inert segment, and (e) a third inert segment, wherein:
the central elastomer comprises liquid silicone rubber (LSR) having a hardness of about 40 to about 65 durometer;
(a) the timed disintegrating matrix comprises about 45 wt % to about 55 wt % PCL, about 27 wt % to about 37 wt % of acid terminated copolymer of DL-lactide and glycolide (50/50 molar ratio) having a viscosity midpoint of about 0.4 dl/g, about 10 wt % to about 22 wt % of ester terminated copolymer of DL-lactide and glycolide (50/50 molar ratio) having a viscosity midpoint of about 0.4 dl/g, about 0.5 wt % to about 5 wt % of polyethylene glycol 100 k, and about 0.005 wt % to about 0.2 wt % color-absorbing dye E172;
(b) the first inert segment comprises about 65 wt % to about 75 wt % PCL, and about 25 wt % to about 35 wt % (BiO)₂CO₃;
(c) the enteric disintegrating matrix comprises about 59 wt % to about 69 wt % HPMCAS, about 29 wt % to about 39 wt % PCL, and about 0.5 wt % to about 5 wt % poloxamer (such as P407);
(d) the second inert segment comprises about 65 wt % to about 75 wt % PCL, and about 25 wt % to about 35 wt % (BiO)₂CO₃; and/or
(e) the third inert segment comprises about 61 wt % to about 71 wt % PCL, about 27 wt % to about 37 wt % copovidone, about 0.2 wt % to about 4 wt % poloxamer and optionally about 0.005 wt % to about 0.2 wt % color-absorbing dye FD&C Blue #1.

In some embodiments, the gastric residence system comprises at least one arm excluding a drug eluting segment, wherein the arm can be attached to a central elastomer, and the arm comprises one or more of: (a) a timed disintegrating matrix, (b) a first inert segment, (c) an enteric disintegrating matrix, (d) a second inert segment, and (e) a third inert segment, wherein:
the central elastomer comprises liquid silicone rubber (LSR) having a hardness of about 45 to about 55 durometer;
(a) the time-dependent disintegrating matrix comprises about 43 wt % to about 47 wt % PCL, about 33 wt % to about 37 wt % of acid terminated copolymer of DL-lactide and glycolide (50/50 molar ratio) having a viscosity midpoint of about 0.4 dl/g, about 15 wt % to about 20 wt % of ester terminated copolymer of DL-lactide and glycolide (50/50 molar ratio) having a viscosity midpoint of about 0.4 dl/g, about 1 wt % to about 3 wt % of polyethylene glycol 100 k, and about 0.01 wt % to about 0.1 wt % color-absorbing dye E172;
(b) the first inert segment comprises about 68 wt % to about 72 wt % PCL, and about 28 wt % to about 32 wt % (BiO)₂CO₃;
(c) the enteric disintegrating matrix comprises about 62 wt % to about 66 wt % HPMCAS, about 32 wt % to about 36 wt % PCL, and about 1 wt % to about 3 wt % poloxamer (such as P407);
(d) the second inert segment comprises about 68 wt % to about 72 wt % PCL, and about 28 wt % to about 32 wt % (BiO)₂CO₃; and/or
(e) the third inert segment comprises about 64 wt % to about 69 wt % PCL, about 30 wt % to about 34 wt % copovidone, about 0.5 wt % to about 2.5 wt % poloxamer and optionally about 0.01 wt % to about 0.1 wt % color-absorbing dye FD&C Blue #1.

In some embodiments, the gastric residence system comprises at least one arm excluding a drug eluting segment, wherein the arm can be attached to a central elastomer, and the arm comprises one or more of: (a) a timed disintegrating matrix, (b) a first inert segment, (c) an enteric disintegrating matrix, (d) a second inert segment, and (e) a third inert segment, wherein:
the central elastomer comprises liquid silicone rubber (LSR) having a hardness of about 45 to about 55 durometer;
(a) the time-dependent disintegrating matrix comprises about 48 wt % to about 52 wt % PCL, about 30 wt % to about 34 wt % of acid terminated copolymer of DL-lactide and glycolide (50/50 molar ratio) having a viscosity midpoint of about 0.4 dl/g, about 14 wt % to about 18 wt % of ester terminated copolymer of DL-lactide and glycolide (50/50 molar ratio) having a viscosity midpoint of about 0.4 dl/g, about 1 wt % to about 3 wt % of polyethylene glycol 100 k, and about 0.01 wt % to about 0.1 wt % color-absorbing dye E172;
(b) the first inert segment comprises about 68 wt % to about 72 wt % PCL, and about 28 wt % to about 32 wt % (BiO)₂CO₃;
(c) the enteric disintegrating matrix comprises about 62 wt % to about 66 wt % HPMCAS, about 32 wt % to about 36 wt % PCL, and about 1 wt % to about 3 wt % poloxamer (such as P407);
(d) the second inert segment comprises about 68 wt % to about 72 wt % PCL, and about 28 wt % to about 32 wt % (BiO)₂CO₃; and/or
(e) the third inert segment comprises about 64 wt % to about 69 wt % PCL, about 30 wt % to about 34 wt % copovidone, about 0.5 wt % to about 2.5 wt % poloxamer and optionally about 0.01 wt % to about 0.1 wt % color-absorbing dye FD&C Blue #1.

In some embodiments, the gastric residence system comprises at least one arm excluding a drug eluting segment, wherein the arm can be attached to a central elastomer, and the arm comprises one or more of: (a) a timed disintegrating matrix, (b) a first inert segment, (c) an enteric disintegrating matrix, (d) a second inert segment, and (e) a third inert segment, wherein:
the central elastomer comprises liquid silicone rubber (LSR) having a hardness of about 50 durometer;
(a) time-dependent disintegrating matrix, the time-dependent disintegrating matrix comprises about 44.95 wt % PCL, about 35 wt % of acid terminated copolymer of DL-lactide and glycolide (50/50 molar ratio) having a viscosity midpoint of about 0.4 dl/g, about 18 wt % of ester terminated copolymer of DL-lactide and glycolide (50/50 molar ratio) having a viscosity midpoint of about 0.4 dl/g, about 2 wt % of polyethylene glycol 100 k and about 0.05 wt % color-absorbing dye E172;
(b) the second inert segment comprises about 70 wt % PCL, and about 30 wt % (BiO)₂CO₃;
(c) the enteric disintegrating matrix comprises about 63.95 wt % HPMCAS, about 33.95 wt % PCL, and about 2 wt % poloxamer (such as P407);
(d) the second inert segment comprises about 70 wt % PCL, and about 30 wt % (BiO)₂CO₃; and/or
(e) the third inert segment comprises about 66.45 wt % PCL, about 32 wt % copovidone, about 1.5 wt % poloxamer and optionally about 0.05 wt % color-absorbing dye FD&C Blue #1.

In some embodiments, the gastric residence system comprises at least one arm excluding a drug eluting segment, wherein the arm can be attached to a central elastomer, and the arm comprises one or more of: (a) a timed disintegrating matrix, (b) a first inert segment, (c) an enteric disintegrating matrix, (d) a second inert segment, and (e) a third inert segment, wherein:
the central elastomer comprises liquid silicone rubber (LSR) having a hardness of about 50 durometer;
(a) time-dependent disintegrating matrix, the time-dependent disintegrating matrix comprises about 49.95 wt % PCL, about 32 wt % of acid terminated copolymer of DL-lactide and glycolide (50/50 molar ratio) having a viscosity midpoint of about 0.4 dl/g, about 16 wt % of ester terminated copolymer of DL-lactide and glycolide (50/50 molar ratio) having a viscosity midpoint of about 0.4 dl/g, about 2 wt % of polyethylene glycol 100 k and about 0.05 wt % color-absorbing dye E172;
(b) the second inert segment comprises about 70 wt % PCL, and about 30 wt % (BiO)₂CO₃;
(c) the enteric disintegrating matrix comprises about 63.95 wt % HPMCAS, about 33.95 wt % PCL, and about 2 wt % poloxamer (such as P407);
(d) the second inert segment comprises about 70 wt % PCL, and about 30 wt % (BiO)₂CO₃; and/or
(e) the third inert segment comprises about 66.45 wt % PCL, about 32 wt % copovidone, about 1.5 wt % poloxamer and optionally about 0.05 wt % color-absorbing dye FD&C Blue #1.

In some embodiments according to any of the gastric residence systems described herein, the gastric residence system comprises at least one arm including a drug eluting segment, wherein the arm further comprises a fourth optional inert segment, wherein the fourth optional inert segment comprises about 65 wt % to about 75 wt % PCL, and about 25 wt % to about 35 wt % (BiO)₂CO₃. In some embodiments, the fourth optional inert segment comprises about 68 wt % to about 72 wt % PCL, and about 28 wt % to about 32 wt % (BiO)₂CO₃. In some embodiments, the fourth optional inert segment comprises about 70 wt % PCL, and about 30 wt % (BiO)₂CO₃.

In any of the above-described embodiments, the arm can be attached to the central elastomer at the first inert segment. That is, the first inert segment is the proximal end of the arm.

The table below provides a listing of the length of each segment in a drug-eluting arm in the gastric residence system. Each range or value below can be considered to be “about” the range or value indicated, or exactly the range or value indicated.

| Segment | Length | |
|---|---|---|
| Drug-eluting segment | 7.8 | mm |
| Third inert segment | 5.3 | mm |
| First, second, or fourth inert segment | 0.5 | mm |
| Enteric disintegrating matrix | 1.85 | mm |
| Time-dependent disintegrating matrix | 1.00 | mm |

The table below provides a listing of the length and thickness of each segment in a drug-eluting arm in the gastric residence system. Each range or value below can be considered to be “about” the range or value indicated, or exactly the range or value indicated.

| | | |
|---|---|---|
| Segment | Length | Thickness |

| | | | |
|---|---|---|---|
| Drug-eluting segment | 7.8 | mm | 3.3 mm |
| Third inert segment | 5.3 | mm | 3.3 mm |
| First, second or fourth inert segment | 0.5 | mm | 3.3 mm |
| Enteric disintegrating matrix | 1.85 | mm | 3.3 mm |
| Time-dependent disintegrating matrix | 1.00 | mm | 3.3 mm |

The table below provides a listing of the length and thickness of each segment in a drug-eluting arm in the gastric residence system. Each range or value below can be considered to be “about” the range or value indicated, or exactly the range or value indicated.

| | | |
|---|---|---|
| Segment | Length | Thickness |

| | | | |
|---|---|---|---|
| Drug-eluting segment | 7.8 | mm | 3.3 mm |
| Third inert segment | 5.3 | mm | 3.1 mm |
| First, second or fourth inert segment | 0.5 | mm | 3.3 mm |
| Enteric disintegrating matrix | 1.85 | mm | 3.3 mm |
| Time-dependent disintegrating matrix | 1.00 | mm | 3.3 mm |

The table below provides a listing of the length of each segment in a drug-free arm in the gastric residence system. Each range or value below can be considered to be “about” the range or value indicated, or exactly the range or value indicated.

| Segment | Length | |
|---|---|---|
| Third inert segment | 13.1 | mm |
| First, second, or fourth inert segment | 0.5 | mm |
| Enteric disintegrating matrix | 1.85 | mm |
| Time-dependent disintegrating matrix | 1.00 | mm |

The table below provides a listing of the length and thickness of each segment in a drug-free arm in the gastric residence system. Each range or value below can be considered to be “about” the range or value indicated, or exactly the range or value indicated.

| | | |
|---|---|---|
| Segment | Length | Thickness |

| | | | |
|---|---|---|---|
| Third inert segment | 13.1 | mm | 3.3 mm |
| First, second or fourth inert segment | 0.5 | mm | 3.3 mm |
| Enteric disintegrating matrix | 1.85 | mm | 3.3 mm |
| Time-dependent disintegrating matrix | 1.00 | mm | 3.3 mm |

The table below provides a listing of the length and thickness of each segment in a drug-free arm in the gastric residence system. Each range or value below can be considered to be “about” the range or value indicated, or exactly the range or value indicated.

| | | |
|---|---|---|
| Segment | Length | Thickness |

| | | | |
|---|---|---|---|
| Third inert segment | 13.1 | mm | 3.1 mm |
| First, second or fourth inert segment | 0.5 | mm | 3.3 mm |
| Enteric disintegrating matrix | 1.85 | mm | 3.3 mm |
| Time-dependent disintegrating matrix | 1.00 | mm | 3.3 mm |

In some embodiments according to any of the systems described herein, the thickness of a segment is determined by the longest straight line within a cross-section in the segment. In some embodiments wherein the cross-section of the segment is a circle, the thickness is defined by the diameter of the circle. In some embodiments, wherein the cross-section of the segment is a square or rectangle, the thickness is defined by the diagonal of the square or rectangle. In some embodiments, wherein the cross-section of the segment is an equilateral triangle, the thickness is defined by the side of the equilateral triangle.

The gastric residence systems above, while described as being risperidone-formulated, are not limited as such, and can be used with other drugs by replacing the segment(s) containing risperidone and/or replacing inert segment(s), with segments containing other drugs.

In some embodiments, the dosage form for administration of risperidone comprises a gastric residence system, wherein the gastric residence system comprises one or two inactive segments. In some embodiments, the gastric residence system comprises a first inactive segment comprising about 66.495 wt % of polycaprolactone (PCL), such as PCL having a viscosity midpoint between about 1.5 dl/g to about 2.1 dl/g, such as Corbion PC17. In some embodiments, the gastric residence system comprises a first inactive segment comprising about, about 32.0 wt % of copovidone, such as VA64. In some embodiments, the gastric residence system comprises a first inactive segment comprising about 1.5 wt % of poly(ethylene glycol)-block-poly(propylene glycol)-block-poly(ethylene glycol) polymers, such as H—(OCH2CH2)x-(O—CH(CH3)CH2)y-(OCH2CH2)z-OH where x and z are about 101 and y is about 56, such as Poloxamer 407 (P407). In some embodiments, the gastric residence system comprises a first inactive segment comprising about 0.005 wt % of iron oxide, such as E172. In some embodiments, the gastric residence system comprises a second inactive segment comprising about 39.995 wt % of polycaprolactone (PCL), such as PCL having a viscosity midpoint between about 1.5 dl/g to about 2.1 dl/g, such as Corbion PC17. In some embodiments, the gastric residence system comprises a second inactive segment comprising about, about 42.0 wt % of copovidone, such as VA64. In some embodiments, the gastric residence system comprises a second inactive segment comprising about 15.0 wt % of polyethylene glycol, such as polyethylene glycol with average molecular weight of 100,000, such as PEO_{100K}. In some embodiments, the gastric residence system comprises a second inactive segment comprising about 3.0 wt % of poly(ethylene glycol)-block-poly(propylene glycol)-block-poly(ethylene glycol) polymers, such as H—(OCH2CH2)x-(O—CH(CH3)CH2)y-(OCH2CH2)z-OH where x and z are about 101 and y is about 56, such as Poloxamer 407 (P407). In some embodiments, the gastric residence system comprises a second inactive segment comprising about 0.005 wt % of iron oxide, such as E172. In some embodiments, the dosage form for administration of risperidone comprises a gastric residence system, wherein the gastric residence system comprises one or two inactive segments. In some embodiments, the gastric residence system comprises a first inactive segment comprising about 66.45 wt % of Corbion PC17, about 32.0 wt % of VA 64, about 1.5 wt % of P407 and about 0.05 wt % of FD&C Blue 1 Aluminum lake. In some embodiments, the gastric residence system comprises a second inactive segment comprising about 39.995 wt % of Corbion PC17, about 42.0 wt % of VA 64, about 15.0 wt % of PEO_{100K}, about 3.0 wt % of P407 and about 0.005 wt % of E172.

In some embodiments, a gastric residence system dosage form for administration of one or more agents can comprise a radiopaque segment, where the segment comprises about 70 wt % of polycaprolactone (PCL), such as PCL having a viscosity midpoint between about 1.5 dl/g to about 2.1 dl/g, such as Corbion PC17. In some embodiments, the gastric residence system comprises a radiopaque segment comprising about 30 wt % of (BiO)₂CO. In some embodiments, the gastric residence system comprises a radiopaque segment comprising about 70 wt % of Corbion PC17, and about 30 wt % of (BiO)₂CO₃.

In some embodiments, a gastric residence system dosage form for administration of risperidone comprises a central elastomer, and a drug-eluting segment comprising about 14 mg of risperidone. In some embodiments, the dosage form comprises a gastric residence system, wherein the gastric residence system comprises a drug-eluting segment comprising about 28 mg of risperidone. In some embodiments, the gastric residence system further comprises a release rate-modulating film comprising about 73.5 wt % of polycaprolactone (PCL), such as PCL having a viscosity midpoint between about 1.5 dl/g to about 2.1 dl/g, such as Corbion PC17. In some embodiments, the release rate-modulating film further comprises about 24.5 wt % of copovidone, such as VA64. In some embodiments, the release rate-modulating film further comprises about 2.0 wt % of Mg stearate. In some embodiments, the gastric residence system further comprises a time-dependent disintegrating matrix comprising about 44.95 wt % of polycaprolactone (PCL), such as PCL having a viscosity midpoint between about 1.5 dl/g to about 2.1 dl/g, such as Corbion PC17. In some embodiments, the time-dependent disintegrating matrix further comprises about 35.0 wt % of an acid terminated copolymer of DL-lactide and glycolide (50/50 molar ratio) having a viscosity midpoint between about 0.32 dl/g to about 0.48 dl/g (such as about 0.4 dl/g), such as PDLG 5004A. In some embodiments, the time-dependent disintegrating matrix further comprises about 18.0 wt % of a copolymer of DL-lactide and glycolide (50/50 molar ratio) having a viscosity midpoint between about 0.32 dl/g to about 0.48 dl/g (such as about 0.4 dl/g), such as PDLG 5004. In some embodiments, the time-dependent disintegrating matrix further comprises about 2.0 wt % of polyethylene glycol, such as polyethylene glycol with average molecular weight of 100,000, such as PEO_{100K}. In some embodiments, the time-dependent disintegrating matrix further comprises about 0.05 wt % of iron oxide, such as E172. In some embodiments, the gastric residence system further comprises a pH-dependent disintegrating matrix comprising about 33.95 wt % of polycaprolactone (PCL), such as PCL having a viscosity midpoint between about 1.5 dl/g to about 2.1 dl/g, such as Corbion PC17. In some embodiments, the pH-dependent disintegrating matrix further comprises about 63.95 wt % of hypromellose acetate succinate, such as HPMCAS-MG. In some embodiments, the pH-dependent disintegrating matrix further comprises about 2.0 wt % of poly(ethylene glycol)-block-poly(propylene glycol)-block-poly(ethylene glycol) polymers, such as H—(OCH2CH2)x-(O—CH(CH3)CH2)y-(OCH2CH2)z-OH where x and z are about 101 and y is about 56, such as Poloxamer 407 (P407). In some embodiments, the pH-dependent disintegrating matrix further comprises about 0.1 wt % of iron oxide, such as E172. In some embodiments, the gastric residence system further comprises one or more inactive segments. In some embodiments, the gastric residence system further comprises a radiopaque segment comprising about 70 wt % of polycaprolactone (PCL), such as PCL having a viscosity midpoint between about 1.5 dl/g to about 2.1 dl/g, such as Corbion PC17. In some embodiments, the radiopaque segment comprises about 30 wt % of (BiO)₂CO₃. In some embodiments, a dosage form for administration of risperidone comprises a gastric residence system, wherein the gastric residence system comprises a central elastomer, and a drug-eluting segment comprising about 14 mg of risperidone. In some embodiments, the dosage form comprises a gastric residence system, wherein the gastric residence system comprises a drug-eluting segment comprising about 28 mg of risperidone. In some embodiments, the gastric residence system further comprises a release rate-modulating film comprising about 73.5 wt % of Corbion PC17, about 24.5 wt % of VA64, and about 2.0 wt % of Mg stearate. In some embodiments, the gastric residence system further comprises a time-dependent disintegrating matrix comprising about 44.95 wt % of Corbion PC17, about 35.0 wt % of PDLG 5004A, about 18.0 wt % of PDLG 5004, about 2.0 wt % of PEO_{100K}, and about 0.05 wt % of E172. In some embodiments, the gastric residence system further comprises a pH-dependent disintegrating matrix comprising about 33.95 wt % of Corbion PC17, about 63.95 wt % of HPMCAS-MG, about 2.0 wt % of P407, and about 0.1 wt % of E172. In some embodiments, the gastric residence system further comprises one or more inactive segments. In some embodiments, the gastric residence system further comprises a radiopaque segment comprising about 70 wt % of Corbion PC17, and about 30 wt % of (BiO)₂CO₃.

In some embodiments, the gastric residence system has three arms comprising a drug-eluting segment and three arms not comprising a drug eluting segment. In some embodiments, the gastric residence system has six arms comprising a drug-eluting segment.

In some embodiments according to any of the systems described herein, the thickness of a segment is determined by the longest straight line within a cross-section in the segment. In some embodiments wherein the cross-section of the segment is a circle, the thickness is defined by the diameter of the circle. In some embodiments, wherein the cross-section of the segment is a square or rectangle, the thickness is defined by the diagonal of the square or rectangle. In some embodiments, wherein the cross-section of the segment is an equilateral triangle, the thickness is defined by the side of the equilateral triangle.

In some embodiments according to any of the systems described herein, the thickness of segments throughout a stellate arm is uniform. In some embodiments according to any of the systems described herein, the thickness of segments throughout a stellate arm is about 2.8 mm to about 3.7 mm, optionally about 3.1 mm to 3.5 mm and more optionally about 3.3 mm.

In some embodiments according to any of the systems described herein, the thickness of one or more segments at the distal end of an arm (furthest away from the stellate core) is smaller than the thickness of the rest of the proximal segments in the arm, wherein optionally the thickness of the proximal segments could be uniform. In some embodiments according to any of the systems described herein, the thickness of most distal segment is about 2.4 mm to about 3.4 mm, wherein the thickness of the rest of the segments in the arm is about 2.8 mm to about 3.7 mm. In some embodiments, the thickness of the most distal segment is about 2.8 mm to about 3.1 mm, wherein the thickness of the rest of the segments in the arm is 3.1 mm to about 3.5 mm. In some embodiments according to any of the systems described herein, the thickness of most distal segment is about 2.9 mm to about 3.2 mm, wherein the thickness of the rest of the segments in the arm is about 3.2 mm to about 3.5 mm. In some embodiments, the thickness of the most distal segment is about 2.9 mm to about 3.15 mm, wherein the thickness of the rest of the segments in the arm is about 3.2 mm to about 3.4 mm. In some embodiments, the thickness of the most distal segment is about 3.1 mm, wherein the thickness of the rest of the segments in the arm is about 3.3 mm.

### Central Elastomer

The central elastomer provides the gastric residence system with the ability to be compacted into a compressed configuration, which can be placed in a capsule or other suitable containing structure for administration to a subject.

In some embodiments, a dosage form for administration of one or more agents comprises a gastric residence system, wherein the gastric residence system comprises a central elastomer comprising a liquid silicone rubber (LSR). In some embodiments, the LSR has a hardness of about 45 to about 60 durometer.

In some embodiments, a dosage form for administration of one or more agents comprises a gastric residence system, wherein the gastric residence system comprises a central elastomer comprising a liquid silicone rubber (LSR). In some embodiments, the LSR has a hardness of about 45 to about 55 durometer.

In some embodiments, a dosage form for administration of one or more agents comprises a gastric residence system, wherein the gastric residence system comprises a central elastomer comprising a liquid silicone rubber (LSR). In some embodiments, the LSR has a hardness of about 60 durometer.

In some embodiments, a dosage form for administration of one or more agents comprises a gastric residence system, wherein the gastric residence system comprises a central elastomer comprising a liquid silicone rubber (LSR). In some embodiments, the LSR has a hardness of about 50 durometer.

### Rate-Modulating Polymer Films

Release-rate modulating polymer films can be coated onto components of gastric residence systems which release agents, such as drugs. Components coated with the release-rate modulating polymer films disclosed herein have substantially the same release-rate properties before and after exposure to heat which occurs during heat-assisted assembly of a gastric residence system. The composition, parameters, advantages, features, applications and release profiles of release-rate modulating polymer films are disclosed in International Patent Application PCT/US2020/059541, which are hereby incorporated in its entirety. In some embodiments, one or more segments of the composite arms (such as a composite arm including the drug-eluting segment or a composite arm excluding the drug-eluting segment) are coated with a release rate-modulating film. In some embodiments, the drug-eluting segment is coated with a release rate-modulating film. In some embodiments, one or more inert segments are coated with a release rate-modulating film. In some embodiments, the release rate-modulating film is applied in an amount of about 0.5% to about 10%, or about 1% to about 5%, such as about 2% to about 4% of the pre-coating weight of the segment (such as drug-eluting segment and/or inactive segment(s)). In some embodiments, the release rate-modulating film is applied in an amount of about 2.3% to about 3%, such as about 2.6%, of the pre-coating weight of the segment (such as drug-eluting segment and/or inactive segment(s)). In some embodiments, the release rate-modulating film is applied in an amount of about 2.4% to about 3.2%, such as about 2.8%, of the pre-coating weight of the segment (such as drug-eluting segment and/or inactive segment(s)).

Various polymers can be used to form the release-rate modulating polymer films, including PCL. In some embodiments, the release-rate modulating polymer films comprises about 68 wt % to about 78 wt % PCL. In some embodiments, the release-rate modulating polymer films comprises about 71 wt % to about 76 wt % PCL. In some embodiments, the release-rate modulating polymer films comprises about 73.5 wt % PCL.

Other excipients can be added to the carrier polymers to modulate the release of agent, such as copovidone (VA64). In some embodiments, the release-rate modulating polymer films comprises about 20 wt % to about 30 wt % VA64. In some embodiments, the release-rate modulating polymer films comprises about 22 wt % to about 27 wt % VA64. In some embodiments, the release-rate modulating polymer films comprises about 24.5 wt % VA64.

The release rate-modulating film can comprise one or more dispersants, such as magnesium stearate. In some embodiments, the release-rate modulating polymer films comprises about 0.5 wt % to about 5 wt % magnesium stearate. In some embodiments, the release-rate modulating polymer films comprises about 1 wt % to about 3 wt % magnesium stearate. In some embodiments, the release-rate modulating polymer films comprises about 2 wt % magnesium stearate.

In some embodiments, the release-rate modulating polymer film comprises about 68 wt % to about 78 wt % PCL, about 20 wt % to about 30 wt % VA64, and about 0.5 wt % to about 5 wt % magnesium stearate. In some embodiments, the release-rate modulating polymer film comprises about 71 wt % to about 76 wt % PCL, about 22 wt % to about 27 wt % VA64, and about 1 wt % to about 3 wt % magnesium stearate. In some embodiments, the release-rate modulating polymer film comprises about 73.5 wt % PCL, about 24.5 wt % VA64, and about 2 wt % magnesium stearate.

Exemplary amounts of the components for the release-rate modulating film are provided in the table below. The amounts are given in approximate weight percent, with the understanding that when ranges are provided, the amounts are chosen so as to add up to 100%.

| Release rate- | | | |
|---|---|---|---|
| modulating film | Formulation 1 | Formulation 2 | Formulation 3 |
| PCL | 67-77 | 71-76 | 73.5 |
| VA64 | 20-30 | 22-27 | 24.5 |
| Mg stearate | 0.5-5 | 1-3 | 2.0 |

### Encapsulation of Gastric Residence System in Capsule

As described above, an example of a stellate system 100 is shown schematically in FIG. 1A and the described configuration permits the system to be folded or compacted at the central elastomer. FIG. 1B shows a folded configuration 190 of the gastric residence system of FIG. 1A (for clarity, only two arms are illustrated in FIG. 1B). When folded, the overall length of the system is reduced by approximately a factor of two, and the system can be conveniently placed in a container such as a capsule or other container suitable for oral administration.

In some embodiments, the capsule comprises a narrower portion (denoted as “capsule bottom” hereafter) and a wider portion (denoted as “capsule cap”, “capsule top” or “capsule sleeve” hereafter), wherein the capsule is closed by sleeving the wider capsule top over the narrower capsule bottom. In some embodiments, the system is oriented in the capsule such that the stellate core is positioned closer to the capsule bottom and wherein the distal tips of the stellate arms (and any circumferential filaments) are positioned closer to the capsule top, i.e. the capsule sleeve covers the distal tips of the stellate arms. In some embodiments, the system is oriented in the capsule such that the stellate core is positioned closer to the capsule top and wherein the distal tips of the stellate arms (and any circumferential filaments) are positioned closer to the capsule bottom, i.e. the capsule sleeve covers the stellate core. In some embodiments, core side sleeving provides better fit with the disintegrating filament stabilizing ring and ensures proper alignment of the stabilizing ring filament within the capsule for full deployment.

In some embodiments, the capsule is size 000, 00, 0, 1, 2, 3, 4, or 5. In some embodiments, the capsule size is 00 EL. In some embodiments, the capsule is an HPMC capsule. In some embodiments, the capsule comprises about any one of: 1%, 2%, 3%, 4%, or 5% titanium oxide. In some embodiments, the capsule is a white opaque HPMC capsule (size 00EL) with 2% titanium oxide. In some embodiments, the capsule is a white opaque HPMC capsule (size 00EL) with 3% titanium oxide.

In some embodiments, the gastric residence system is assembled and then placed into an appropriate sized capsule as described in Example 1 of International Patent Application PCT/US2020/059541.

In some embodiments, the gastric residence system is assembled and then placed into an appropriate sized capsule as described in International Patent Application PCT/US2020/023704.

In some embodiments, the gastric residence system is assembled and then placed into an appropriate sized capsule as described in International Patent Application PCT/US2020/023710.

The entire contents of International Applications PCT/US2020/059541, PCT/US2020/023704, PCT/US2020/023710 are hereby incorporated by reference herein.

### Extended Release of Drug Dosage Forms

In some embodiments according to any one of the risperidone dosage forms described herein, the gastric residence system allows for extended release of risperidone (such as, including risperidone and any of its active metabolite forms).

In one embodiment, administration of a gastric residence dosage form comprising 14 mg risperidone to a human results in a plasma level of at least about 7.0 ng/mL at about 24 hours. In one embodiment, administration of a gastric residence dosage form comprising 14 mg risperidone to a human results in a plasma level of at least about 6.5 ng/mL at about 48 hours. In one embodiment, administration of a gastric residence dosage form comprising 14 mg risperidone to a human results in a plasma level of at least about 4.5 ng/mL at about 72 hours. In one embodiment, administration of a gastric residence dosage form comprising 14 mg risperidone to a human results in a plasma level of at least about 3.0 ng/mL at about 96 hours. In one embodiment, administration of a gastric residence dosage form comprising 14 mg risperidone to a human results in a plasma level of at least about 2.5 ng/mL at about 120 hours. In one embodiment, administration of a gastric residence dosage form comprising 14 mg risperidone to a human results in a plasma level of at least about 2.0 ng/mL at about 144 hours.

In one embodiment, administration of a gastric residence dosage form comprising 14 mg risperidone to a human results in a plasma level of no more than about 27.0 ng/mL at about 24 hours. In one embodiment, administration of a gastric residence dosage form comprising 14 mg risperidone to a human results in a plasma level of no more than about 22.5 ng/mL at about 48 hours. In one embodiment, administration of a gastric residence dosage form comprising 14 mg risperidone to a human results in a plasma level of no more than about 21.5 ng/mL at about 72 hours. In one embodiment, administration of a gastric residence dosage form comprising 14 mg risperidone to a human results in a plasma level of no more than about 21.0 ng/mL at about 96 hours. In one embodiment, administration of a gastric residence dosage form comprising 14 mg risperidone to a human results in a plasma level of no more than about 20.0 ng/mL at about 120 hours. In one embodiment, administration of a gastric residence dosage form comprising 14 mg risperidone to a human results in a plasma level of no more than about 17.0 ng/mL at about 144 hours.

In one embodiment, administration of a gastric residence dosage form comprising 14 mg risperidone to a human results in a plasma level of between about 7.0 ng/mL to about 27.0 ng/mL at about 24 hours. In one embodiment, administration of a gastric residence dosage form comprising 14 mg risperidone to a human results in a plasma level of between about 6.5 ng/mL to about 22.5 ng/mL at about 48 hours. In one embodiment, administration of a gastric residence dosage form comprising 14 mg risperidone to a human results in a plasma level of between about 4.5 ng/mL to about 21.5 ng/mL at about 72 hours. In one embodiment, administration of a gastric residence dosage form comprising 14 mg risperidone to a human results in a plasma level of between about 3.0 ng/mL to about 21.0 ng/mL at about 96 hours. In one embodiment, administration of a gastric residence dosage form comprising 14 mg risperidone to a human results in a plasma level of between about 2.5 ng/mL to about 20.0 ng/mL at about 120 hours. In one embodiment, administration of a gastric residence dosage form comprising 14 mg risperidone to a human results in a plasma level of between about 2.0 ng/mL to about 17.0 ng/mL at about 144 hours.

In one embodiment, administration of a gastric residence dosage form comprising 28 mg risperidone to a human results in a plasma level of at least about 20.0 ng/mL at about 24 hours. In one embodiment, administration of a gastric residence dosage form comprising 28 mg risperidone to a human results in a plasma level of at least about 16.0 ng/mL at about 48 hours. In one embodiment, administration of a gastric residence dosage form comprising 28 mg risperidone to a human results in a plasma level of at least about 14.5 ng/mL at about 72 hours. In one embodiment, administration of a gastric residence dosage form comprising 28 mg risperidone to a human results in a plasma level of at least about 12.0 ng/mL at about 96 hours. In one embodiment, administration of a gastric residence dosage form comprising 28 mg risperidone to a human results in a plasma level of at least about 9.0 ng/mL at about 120 hours. In one embodiment, administration of a gastric residence dosage form comprising 28 mg risperidone to a human results in a plasma level of at least about 6.0 ng/mL at about 144 hours.

In one embodiment, administration of a gastric residence dosage form comprising 28 mg risperidone to a human results in a plasma level of no more than about 39.0 ng/mL at about 24 hours. In one embodiment, administration of a gastric residence dosage form comprising 28 mg risperidone to a human results in a plasma level of no more than about 39.0 ng/mL at about 48 hours. In one embodiment, administration of a gastric residence dosage form comprising 28 mg risperidone to a human results in a plasma level of no more than about 38.0 ng/mL at about 72 hours. In one embodiment, administration of a gastric residence dosage form comprising 28 mg risperidone to a human results in a plasma level of no more than about 35.0 ng/mL at about 96 hours. In one embodiment, administration of a gastric residence dosage form comprising 28 mg risperidone to a human results in a plasma level of no more than about 30.0 ng/mL at about 120 hours. In one embodiment, administration of a gastric residence dosage form comprising 28 mg risperidone to a human results in a plasma level of no more than about 25.0 ng/mL at about 144 hours.

In one embodiment, administration of a gastric residence dosage form comprising 28 mg risperidone to a human results in a plasma level of between about 20.0 ng/mL to about 39.0 ng/mL at about 24 hours. In one embodiment, administration of a gastric residence dosage form comprising 28 mg risperidone to a human results in a plasma level of between about 16.0 ng/mL to about 39.0 ng/mL at about 48 hours. In one embodiment, administration of a gastric residence dosage form comprising 28 mg risperidone to a human results in a plasma level of between about 14.5 ng/mL to about 38.0 ng/mL at about 72 hours. In one embodiment, administration of a gastric residence dosage form comprising 28 mg risperidone to a human results in a plasma level of between about 12.0 ng/mL to about 35.0 ng/mL at about 96 hours. In one embodiment, administration of a gastric residence dosage form comprising 28 mg risperidone to a human results in a plasma level of between about 9.0 ng/mL to about 30.0 ng/mL at about 120 hours. In one embodiment, administration of a gastric residence dosage form comprising 28 mg risperidone to a human results in a plasma level of between about 6.0 ng/mL to about 25.0 ng/mL at about 144 hours.

In any of these embodiments, administration of the gastric residence dosage form can occur after administration of immediate release risperidone for at least 7 days, such as administration of 2 mg of immediate release risperidone for at least about 7 days, or administration of 4 mg of immediate release risperidone for at least about 7 days.

### EXAMPLES

The disclosure is further illustrated by the following non-limiting examples.

### Example 1: Risperidone Dosage Form (Extended-Release Gastric Residence System)

In this Example, a dosage form according to the present invention includes a gastric residence system, the gastric residence system is formulated to include risperidone.

The gastric residence system includes a central elastomer that provides the gastric residence system with the ability to be compacted into a compressed configuration. The gastric residence system illustrated in this Example is another different arrangement of the “star” configuration. In an example of the risperidone-formulated gastric residence system, the stellate contains 6 arms each comprising a drug-eluting segment.

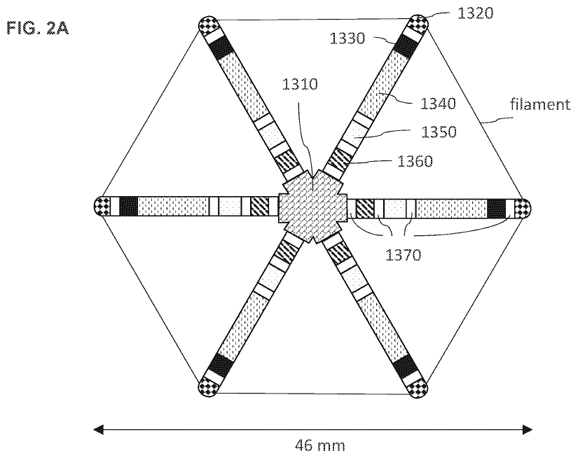

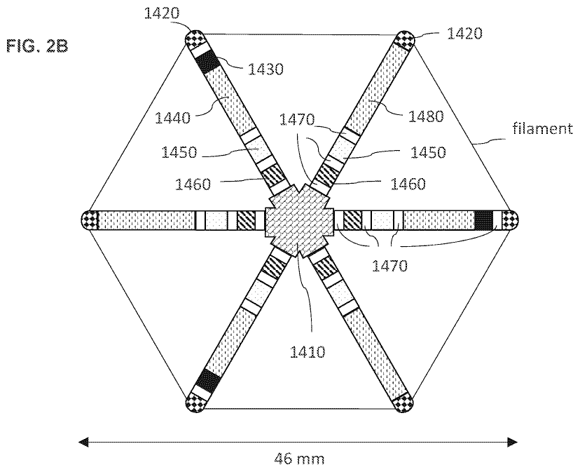

FIG. 2A is labelled to show the various elements of this configuration. The system 1300 comprises a central elastomeric core 1310 which is in the shape of an “asterisk” having six short branches. A segment 1370 of the arm is attached to one short asterisk branch. The segment 1370 is followed by a segment 1360, a second segment 1370, a segment 1350, a third segment 1370, a segment 1340, a segment 1330, and a forth segment 1370 in sequence. The distal end of each arm has segment 1320.

The gastric residence system has an average size of about 46 mm and each segment has a length ranging from about 0.5 mm to about 8.0 mm. Table I below provides a listing of the length of each segment in the gastric residence system. Each range or value below can be considered to be “about” the range or value indicated, or exactly the range or value indicated.

TABLE I

| Segment | Length | |
|---|---|---|
| 1320 | 4 | mm |
| 1330 | 2.4 | mm |
| 1340 | ~8 | mm |
| 1350 | 1.85 | mm |
| 1360 | 1.0 | mm |
| 1370 | 0.5 | mm |

The gastric residence system has an average size of about 46 mm and each segment has a length ranging from about 0.5 mm to about 8.0 mm. Table IA below provides a listing of the length of each segment in an exemplary gastric residence system. Each range or value below can be considered to be “about” the range or value indicated, or exactly the range or value indicated.

TABLE IA

| Segment | Length | |
|---|---|---|
| 1320 | 4 | mm |
| 1330 | 2.4 | mm |
| 1340 | 5.6 | mm |
| 1350 | 1.85 | mm |
| 1360 | 1.0 | mm |
| 1370 | 0.5 | mm |

The central elastomeric core 1310 comprises a liquid silicone rubber (LSR) having a hardness of 50 durometer.

In this example, the dosage form provided here contains 6 arms each comprising a drug-eluting segment, wherein the dosage form comprises about 28 mg of risperidone for administration. Risperidone is included in a carrier polymer-agent segment 1330 (e.g., a drug-eluting segment). The drug-eluting segment comprises about 35.0 wt % of risperidone, about 55.9 wt % of Corbion PC17, about 5.0 wt % of VA64, about 3.0 wt % of P407, about 0.5 wt % of Vitamin E succinate, about 0.5 wt % of SiO₂, and about 0.1 wt % of pigment. The pigment includes about 0.05% of FD&C Yellow 5 Alum lake (14-16%) and about 0.05% of FD&C Blue 1 Alum lake (11-13%). Also contemplated in the present application are variations of this dosage form with increased numbers and/or lengths of the drug-eluting segments to achieve higher doses of the drug, for example, risperidone.

Moreover, each arm comprises inactive segment 1340. The inactive segment 1340 comprises about 66.45 wt % of Corbion PC17, about 32.0 wt % of VA 64, about 1.5 wt % of P407 and about 0.05 wt % of FD&C Blue 1 Aluminum lake.

The gastric residence system further includes a time-dependent disintegrating matrix or linker, referred as the segment 1360, as well as a pH-dependent disintegrating matrix or linker, referred as the segment 1350. In addition, the gastric residence system includes a structural segment 1370.

The time-dependent disintegrating matrix (segment 1360) comprises about 44.95 wt % Corbion PC17, about 35 wt % of acid terminated copolymer of DL-lactide and glycolide (PDLG5004A), about 18 wt % of copolymer of DL-lactide and glycolide (PDLG5004), about 2 wt % of polyethylene glycol 100 k and about 0.05 wt % color-absorbing dye E172. The pH-dependent disintegrating matrix (segment 1350) comprises about 63.95 wt % HPMCAS, about 33.95 wt % Corbion PC17, about 2 wt % P407 and about 0.1 wt % color-absorbing dye E172. The structural segment 1370 can be a radiopaque-PCL segment, comprising about 70 wt % PCL, and about 30 wt % (BiO)₂CO₃.

Segment 1320, at the distal end of each arm, is a third disintegrating matrix, to which a filament is also optionally attached, where the filament thereby circumferentially connects the arms. The third disintegrating matrix (Segment 1320) comprises about 64.9 wt % HPMCAS, about 30 wt % PCL, about 2.5 wt % propylene glycol, about 2.5 wt % stearic acid, and about 0.1 wt % iron oxide (for example about 0.025% ferrosoferric oxide and about 0.075% FD&C Red 40).

In the gastric residence system, each drug arm is coated by a release rate-modulating film. Specifically, the coating comprises about 73.5 wt % of Corbion PC17, about 24.5 wt % of VA64, and about 2.0 wt % of Mg stearate, and is applied in an amount of about 4.5% of the pre-coating weight of the segment (i.e., segments 1320, 1330 and 1340).

The gastric residence system is assembled and then placed into an appropriate sized capsule as described in Example 1 of International Patent Application PCT/US2020/059541. The dosage form described here differs from a gastric residence system previously described in International Patent Application No. PCT/US2020/059541, and other gastric residence systems previously designated as LYN-005.

In another example of the risperidone-formulated gastric residence system, the stellate contains 3 arms each comprising a drug-eluting segment, and 3 arms not comprising a drug-eluting segment. Also contemplated in this application are other gastric residence systems containing 6 arms of which either 1, 2, 3, 4, 5, to 6 arms comprise a drug-eluting segment.

FIG. 2B is labelled to show the various elements of this configuration. The system 1400 comprises a central elastomeric core 1410 which is in the shape of an “asterisk” having six short branches.

For an arm containing a drug-eluting segment, a segment 1470 of the arm is attached to one short asterisk branch. The segment 1470 is followed by a segment 1460, a second segment 1470, a segment 1450, a third segment 1470, a segment 1440, a segment 1430, and a forth segment 1470 in sequence. The distal end of each drug-containing arm has segment 1420.

For an arm not containing a drug-eluting segment, a segment 1470 of the arm is attached to one short asterisk branch. The segment 1470 is followed by a segment 1460, a second segment 1470, a segment 1450, a third segment 1470, and a segment 1480. The distal end of each drug-free arm has segment 1420.

The gastric residence system has an average size of about 46 mm and each segment has a length ranging from about 0.5 mm to about 10.9 mm. Table II below provides a listing of the length of each segment in the gastric residence system. Each range or value below can be considered to be “about” the range or value indicated, or exactly the range or value indicated.

TABLE II

| Segment | Length | |
|---|---|---|
| 1420 | 4 | mm |
| 1430 | 2.4 | mm |
| 1440 | ~8 | mm |
| 1450 | 1.85 | mm |
| 1460 | 1.0 | mm |
| 1470 | 0.5 | mm |
| 1480 | ~10.9 | mm |

The gastric residence system has an average size of about 46 mm and each segment has a length ranging from about 0.5 mm to about 10.9 mm. Table IIA below provides a listing of the length of each segment in an exemplary gastric residence system. Each range or value below can be considered to be “about” the range or value indicated, or exactly the range or value indicated.

TABLE IIA

| Segment | Length | |
|---|---|---|
| 1420 | 4 | mm |
| 1430 | 2.4 | mm |
| 1440 | ~5.6 | mm |
| 1450 | 1.85 | mm |
| 1460 | 1.0 | mm |
| 1470 | 0.5 | mm |
| 1480 | ~8 | mm |

The central elastomeric core 1410 comprises a liquid silicone rubber (LSR) having a hardness of 50 durometer.

In this example, the dosage form provided here contains 6 arms, half of which (3 arms) each comprises a drug-eluting segment, wherein the dosage form comprises about 14 mg of risperidone for administration. The 3 drug-containing arms can be arranged at each alternate arm around the stellate. Risperidone is included in a carrier polymer-agent segment 1430 (e.g., a drug-eluting segment). The drug-eluting segment comprises about 35.0 wt % of risperidone, about 55.9 wt % of Corbion PC17, about 5.0 wt % of VA64, about 3.0 wt % of P407, about 0.5 wt % of Vitamin E succinate, about 0.5 wt % of SiO₂, and about 0.1 wt % of pigment. The pigment includes about 0.05% of FD&C Yellow 5 Alum lake (14-16%) and about 0.05% of FD&C Blue 1 Alum lake (11-13%). Also contemplated in the present application are variations of this dosage form with increased numbers and/or lengths of the drug-eluting segments to achieve higher doses of the drug, for example, risperidone.

Moreover, each arm comprises inactive segment (segment 1440 for drug-containing arms and segment 1480 for drug-free arms). The inactive segment 1440 or 1480 each comprises about 66.45 wt % of Corbion PC17, about 32.0 wt % of VA 64, about 1.5 wt % of P407 and about 0.05 wt % of FD&C Blue 1 Aluminum lake.

The gastric residence system further includes a time-dependent disintegrating matrix or linker, referred as the segment 1460, as well as a pH-dependent disintegrating matrix or linker, referred as the segment 1450. In addition, the gastric residence system includes a structural segment 1470.

The time-dependent disintegrating matrix (segment 1460) comprises about 44.95 wt % Corbion PC17, about 35 wt % of acid terminated copolymer of DL-lactide and glycolide (PDLG5004A), about 18 wt % of copolymer of DL-lactide and glycolide (PDLG5004), about 2 wt % of polyethylene glycol 100 k and about 0.05 wt % color-absorbing dye E172. The pH-dependent disintegrating matrix (segment 1450) comprises about 63.95 wt % HPMCAS, about 33.95 wt % Corbion PC17, about 2 wt % P407 and about 0.1 wt % color-absorbing dye E172. The structural segment 1470 can be a radiopaque-PCL segment, comprising about 70 wt % PCL, and about 30 wt % (BiO)₂CO₃.

Segment 1420, at the distal end of each arm, is a third disintegrating matrix, to which a filament is also optionally attached, where the filament thereby circumferentially connects the arms. The third disintegrating matrix (Segment 1420) comprises about 64.9 wt % HPMCAS, about 30 wt % PCL, about 2.5 wt % propylene glycol, about 2.5 wt % stearic acid, and about 0.1 wt % iron oxide (for example about 0.025% ferrosoferric oxide and about 0.075% FD&C Red 40).

In the gastric residence system, each drug arm is coated by a release rate-modulating film. Specifically, the coating comprises about 73.5 wt % of Corbion PC17, about 24.5 wt % of VA64, and about 2.0 wt % of Mg stearate, and is applied in an amount of about 4.5% of the pre-coating weight of the segment (i.e., segments 1420, 1430, 1440 and 1480).

The gastric residence system is assembled and then placed into an appropriate sized capsule as described in Example 1 of International Patent Application PCT/US2020/059541. The dosage form described here differs from a gastric residence system previously described in International Patent Application No. PCT/US2020/059541, and other gastric residence systems previously designated as LYN-005.

### Study in Humans

Patients undergoing treatment for schizophrenia or schizoaffective disorder received immediate-release (IR) risperidone tablets (2 mg or 4 mg, based on patient's current APD dose) for 15 days, and then were randomized 3:1 to receive IR risperidone-matched placebo or a risperidone-containing gastric residence system as described herein (14 mg or 28 mg risperidone), or a gastric residence system matched placebo and IR risperidone (2 mg or 4 mg; 4 patients per group) for 3 weeks. The gastric residence system was administered once weekly. IR risperidone was administered once daily.

Following gastric residence system administration, systemic exposure to risperidone active moiety (risperidone and 9-hydroxyrisperidone combined) increased with increasing dose. Exposure was observed throughout the dosing intervals with peak concentration generally observed within the first 3 days of dosing.

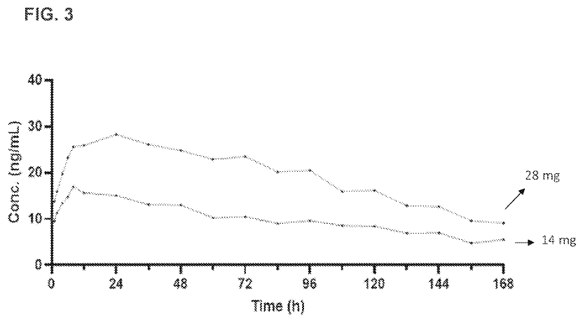

The dosage forms were administered to humans, and plasma samples were collected from participants. The pharmacokinetics of the formulation of the risperidone dosage forms in the subjects are shown in FIG. 3 (upper curve: 28 mg dosage form; lower curve: 14 mg dosage form). Specifically, the plasma concentration of the active moiety, risperidone and 9-hydroxy-risperidone (the active metabolite of risperidone) was plotted over the course of 7 days.

This formulation of the risperidone extended-release gastric residence system demonstrated drug release over seven days for both the 14 mg and 28 mg risperidone dosage forms described in this Example 1.

### Example 2: Three-Week Study of Risperidone Dosage Form (Extended-Release Gastric Residence System)

Methods: A multiple-dose, randomized, parallel group, placebo-controlled, study was conducted that enrolled 32 clinically stable patients with a primary diagnosis of schizophrenia or schizoaffective disorder. Patients received immediate-release (IR) risperidone tablets (2 mg or 4 mg, based on patient's current antipsychotic dose) for a 13-day lead-in period (the “IR lead-in”) and then were randomized 3:1 to receive IR risperidone-matched placebo and a risperidone gastric residence system with the appropriate risperidone loading as described in Example 1 (14 mg or 28 mg risperidone; 12 patients per group) or risperidone gastric residence system matched placebo and IR risperidone (2 mg or 4 mg; 4 patients per group) for 3 weeks.

The risperidone gastric residence system dose for patients receiving the 2 mg/day IR lead-in was 14 mg. The risperidone gastric residence system dose for patients receiving the 4 mg/day IR lead-in was 28 mg. Patients in the risperidone gastric residence system matched placebo/IR risperidone group received the same IR dose they received during the IR lead-in.

The risperidone gastric residence system was administered once weekly (total of 3 doses). IR risperidone was administered once daily. Primary endpoints were pharmacokinetics after administration of risperidone gastric residence system capsules and after IR risperidone, and the incidence of adverse events (AEs). The secondary endpoint was pharmacokinetics after switching from IR risperidone to risperidone gastric residence system. Pharmacokinetics analyses were done using a non-compartmental model.

Plasma samples were analyzed by LCMS for risperidone and its active metabolite, 9-OH-risperidone. The sum of risperidone and 9-OH-risperidone is referred to as the risperidone active moiety. Pharmacokinetic analysis compared active moiety plasma concentrations observed during the 3-week dosing period with concentrations observed during the final day of the IR lead-in (Day −1).

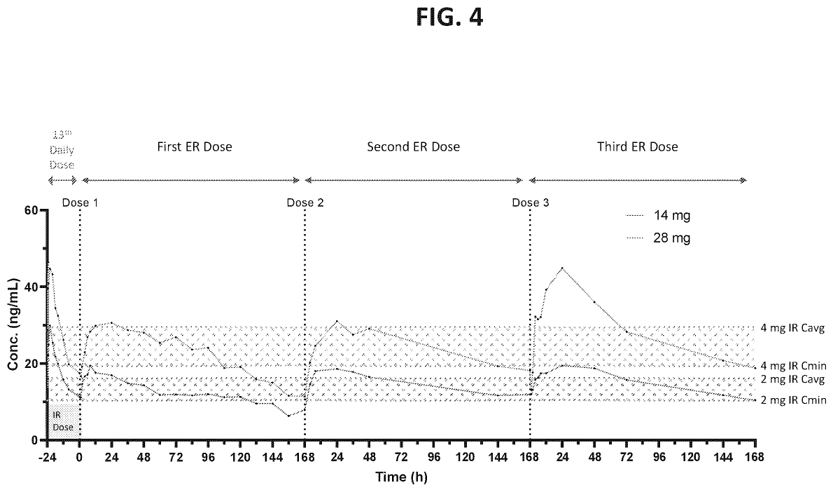

Results: Following risperidone gastric residence system administration, systemic exposure to risperidone active moiety (the concentrations of risperidone and 9-hydroxyrisperidone combined) increased with increasing risperidone gastric residence system dose. Exposure was observed throughout the dosing intervals with peak concentration generally observed within the first 3 days of dosing. Peak exposures from risperidone gastric residence system were lower than with IR risperidone; see FIG. 4, Day −1 (In FIG. 4, in the 14 mg group, n=12; one patient withdrew at Dose 1, 24 h postdose; one patient withdrew after Dose 3, 4 h postdose; and in the 28 mg group, n=11; one patient withdrew after Dose 2, 48 h postdose; one patient was excluded due to unexplained erratic absorption). Inspection of predose concentrations suggested steady-state was attained prior to the third risperidone gastric residence system dose.

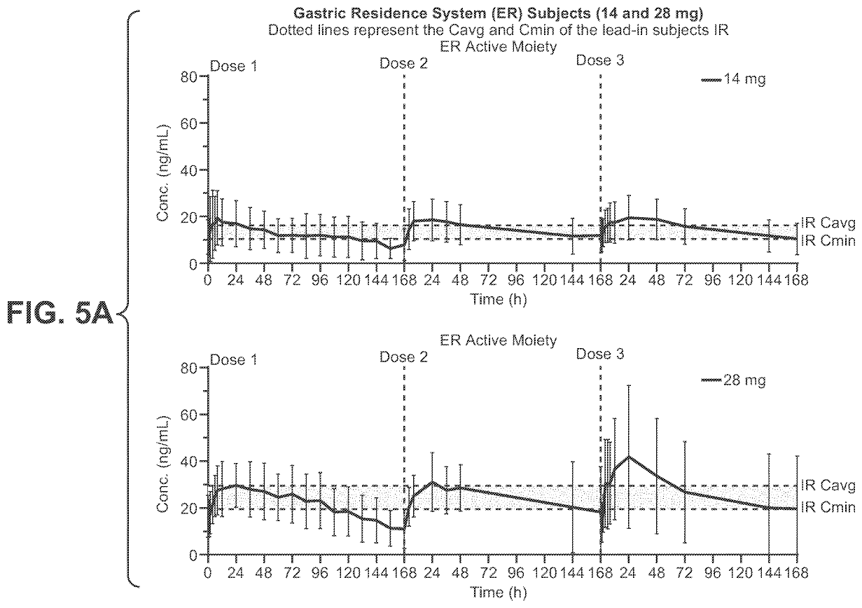

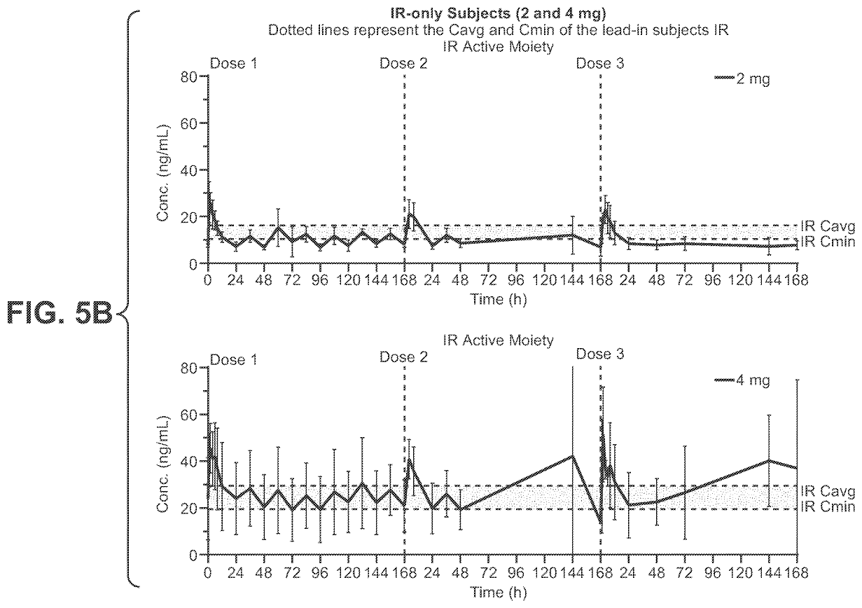

Active moiety plasma concentrations in patients receiving risperidone gastric residence system generally remained above or near the steady-state trough concentration observed during the IR lead-in, i.e., at t=0; see FIG. 4 and FIG. 5A. (In FIG. 4 and FIG. 5A, in the 14 mg group, n=12; one patient withdrew at Dose 1, 24 h postdose; one patient withdrew after Dose 3, 4 h postdose. In FIG. 5A, in the 28 mg group, n=11; one patient withdrew after Dose 2, 48 h postdose; one patient was excluded due to unexplained erratic absorption).

Subjects receiving risperidone gastric residence system-matched placebo and IR risperidone displayed active moiety concentrations consistent with values observed during the IR lead-in; see FIG. 5B. (In FIG. 5B, in the 2 mg group, n=4; in the 4 mg group, n=4, one patient withdrew after Dose 1, 96 h postdose.

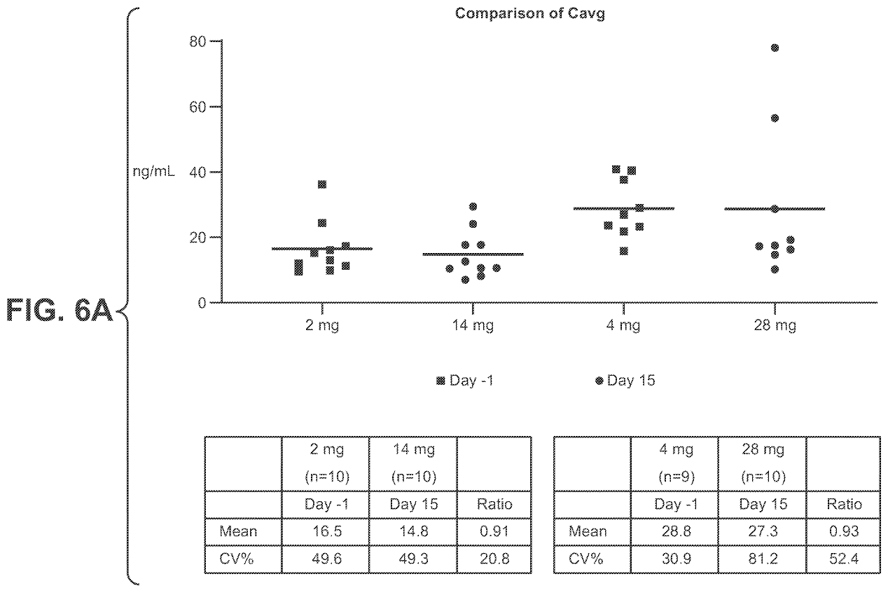

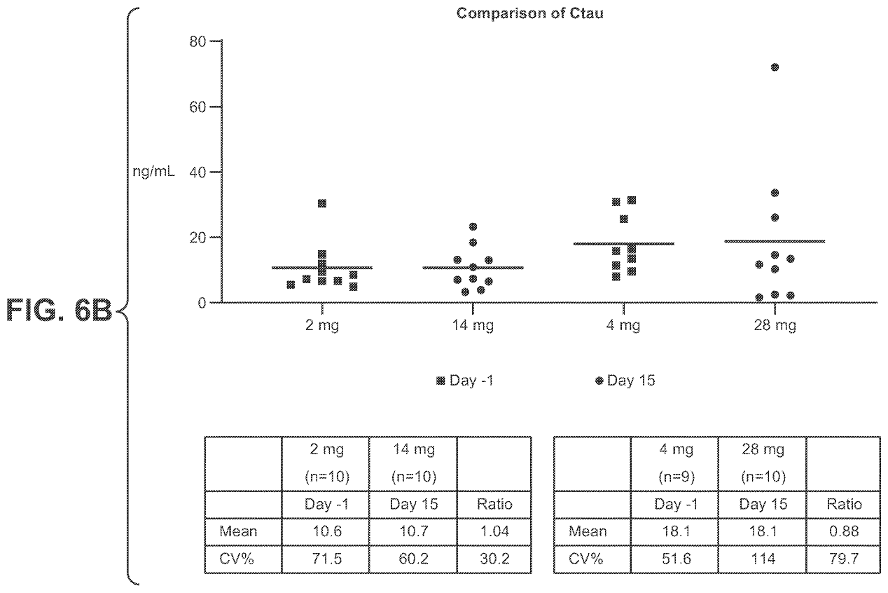

Exposure to active moiety in subjects receiving risperidone gastric residence system and IR risperidone-matched placebo was assessed by comparing the average concentration (Cavg) and the trough concentration (Ctau) for the one-week period following administration of the third risperidone gastric residence system dose and for the last day of the IR lead-in. Cavg and Ctau values were generally similar for IR and risperidone gastric residence system administration at both dose levels (i.e., 2 mg IR/14 mg risperidone gastric residence system) and 4 mg/IR/28 mg risperidone gastric residence system); see FIG. 6A and FIG. 6B.

The risperidone gastric residence system was well tolerated with ˜85% of subjects completing all three dosages. No severe or SAEs were reported.

### Example 3: Risperidone Dosage Form (Extended-Release Gastric Residence System)

In this Example, a dosage form according to the present invention includes a gastric residence system, the gastric residence system is formulated to include risperidone.

The gastric residence system includes a central elastomer that provides the gastric residence system with the ability to be compacted into a compressed configuration. The gastric residence system illustrated in this Example is another different arrangement of the “star” configuration. In an example of the risperidone-formulated gastric residence system, the stellate contains 6 arms each comprising a drug-eluting segment.

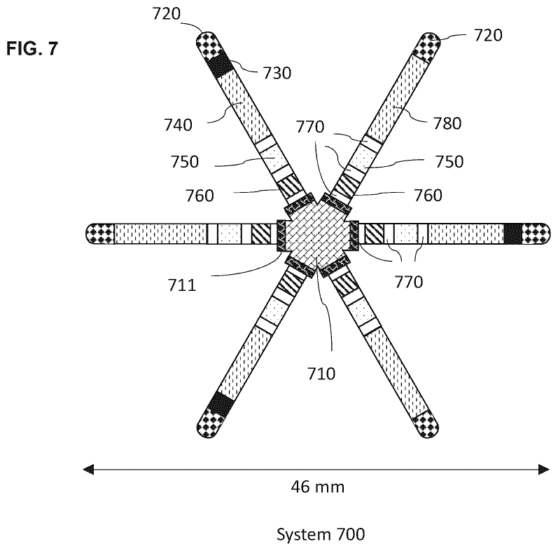

FIG. 7 is labelled to show the various elements of this configuration. The system 700 comprises a central elastomeric core 710 which is in the shape of an “asterisk” having six short branches. Segment 711 is an inert polycaprolactone linker segment at the end of each branch of the core which facilitates the attachment of the arms.

For an arm containing a drug-eluting segment, a segment 770 of the arm is attached to one short asterisk branch. The segment 770 is followed by a segment 760, a second segment 770, a segment 750, a third segment 770, a segment 740, and a segment 730 in sequence. The distal end of each arm has segment 720.

For an arm not containing a drug-eluting segment, a segment 1470 of the arm is attached to one short asterisk branch. The segment 770 is followed by a segment 760, a second segment 770, a segment 750, a third segment 770, and a segment 780. The distal end of each drug-free arm has segment 720.

The gastric residence system has an average size of about 46 mm and each segment has a length ranging from about 0.5 mm to about 8.0 mm. Table A below provides a listing of the length of each segment in the gastric residence system. Each range or value below can be considered to be “about” the range or value indicated, or exactly the range or value indicated.

TABLE A

| Segment | Length | |
|---|---|---|
| 720 | 4 | mm |
| 730 | 2.4 | mm |
| 740 | 5.6 | mm |
| 750 | 1.85 | mm |
| 760 | 1.0 | mm |
| 770 | 0.5 | mm |
| 780 | 8.0 | mm |

In the gastric residence system, the cross-section of an arm could be an oval, a circle, a rectangle, a square, or a triangle. In one exemplary system, the cross-section of an arm could be an equilateral triangle, wherein the side of the triangle is about 3.0 mm-3.5 mm, such as about 3.3 mm. In one exemplary system, the cross-section of an arm could be a square or a rectangle, wherein the diagonal of the square or rectangle is about 3.0 mm-3.5 mm, such as about 3.3 mm. In one exemplary system, the cross-section of an arm could be a circle, wherein the diameter of the circle is about 3.0 mm-3.5 mm, such as about 3.3 mm.

The central elastomeric core 710 comprises a liquid silicone rubber (LSR) having a hardness of 50 durometer.

In this example, the dosage form provided here contains 6 arms, half of which (3 arms) each comprises a drug-eluting segment, wherein the dosage form comprises about 14 mg of risperidone for administration. The 3 drug-containing arms can be arranged at each alternate arm around the stellate. Risperidone is included in a carrier polymer-agent segment 730 (e.g., a drug-eluting segment). The drug-eluting segment comprises about 35.0 wt % of risperidone, about 55.9 wt % of Corbion PC17, about 5.0 wt % of VA64, about 3.0 wt % of P407, about 0.5 wt % of Vitamin E succinate, about 0.5 wt % of SiO₂, and about 0.1 wt % of pigment. The pigment includes about 0.05% of FD&C Yellow 5 Alum lake (14-16%) and about 0.05% of FD&C Blue 1 Alum lake (11-13%). Also contemplated in the present application are variations of this dosage form with increased numbers and/or lengths of the drug-eluting segments to achieve higher doses of the drug, for example, risperidone.

Moreover, each arm comprises two inactive segments. The first inactive segment being indicated segment 740 for drug-containing arms and segment 780 for drug-free arms. The first inactive segment 740 or 780 each comprises about 66.45 wt % of Corbion PC17, about 32.0 wt % of VA 64, about 1.5 wt % of P407 and about 0.05 wt % of FD&C Blue 1 Aluminum lake. The second inactive segment, 720, at the distal end of each arm, to which a filament can optionally be attached, comprises about 39.995 wt % of Corbion PC17, about 42.0 wt % of VA 64, about 15.0 wt % of PEO100 k, about 3.0 wt % of P407 and about 0.05 wt % of Ferrosoferric oxide E172.

The gastric residence system further includes a time-dependent disintegrating matrix or linker, referred as the segment 760, as well as a pH-dependent disintegrating matrix or linker, referred as the segment 750. In addition, the gastric residence system includes a structural segment 770.

The time-dependent disintegrating matrix (segment 760) comprises about 44.95 wt % Corbion PC17, about 35 wt % of acid terminated copolymer of DL-lactide and glycolide (PDLG5004A), about 18 wt % of copolymer of DL-lactide and glycolide (PDLG5004), about 2 wt % of polyethylene glycol 100 k and about 0.05 wt % color-absorbing dye E172. The pH-dependent disintegrating matrix (segment 750) comprises about 63.95 wt % HPMCAS, about 33.95 wt % Corbion PC17, about 2 wt % P407 and about 0.1 wt % color-absorbing dye E172. The structural segment 770 can be a radiopaque-PCL segment, comprising about 70 wt % PCL, and about 30 wt % (BiO)₂CO₃.

In the gastric residence system, each drug arm is coated by a release rate-modulating film. Specifically, the coating comprises about 73.5 wt % of Corbion PC17, about 24.5 wt % of VA64, and about 2.0 wt % of Mg stearate, and is applied in an amount of about 2.3% to about 3%, such as about 2.6%, of the pre-coating weight of the segment (i.e., segments 720, 730, 740 and 780).

The gastric residence system is assembled and then placed into an appropriate sized capsule as described in Example 1 of International Patent Application PCT/US2020/059541. The dosage form described here differs from a gastric residence system previously described in International Patent Application No. PCT/US2020/059541, and other gastric residence systems previously designated as LYN-005.

In another example of the risperidone-formulated gastric residence system, the stellate contains 6 arms each comprising a drug-eluting segment. Also contemplated in this application are other gastric residence systems containing 6 arms of which either 1, 2, 3, 4, 5, or 6 arms comprise a drug-eluting segment.

The described gastric residence systems, while shown as being risperidone-formulated, is not limited as such, and can be used with other drugs by replacing the segment(s) containing risperidone and/or replacing inert segment(s), with segments containing other drugs.

### Example 4: Risperidone Dosage Form (Extended-Release Gastric Residence System)

In this Example, a dosage form according to the present invention includes a gastric residence system, the gastric residence system is formulated to include risperidone.

The gastric residence system includes a central elastomer that provides the gastric residence system with the ability to be compacted into a compressed configuration. The gastric residence system illustrated in this Example is another different arrangement of the “star” configuration. In an example of the risperidone-formulated gastric residence system, the stellate contains 6 arms each comprising a drug-eluting segment.

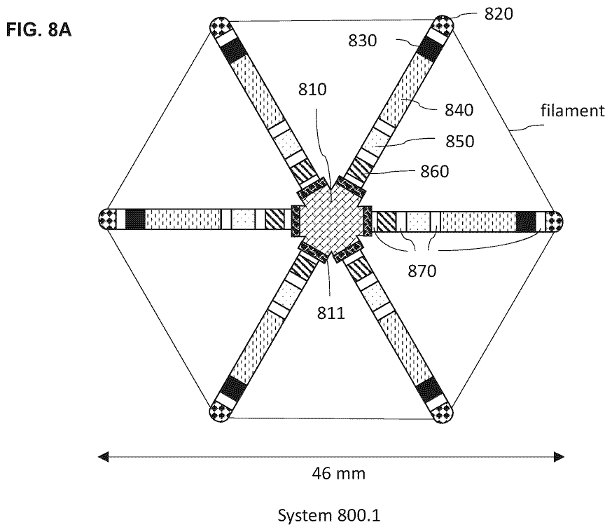

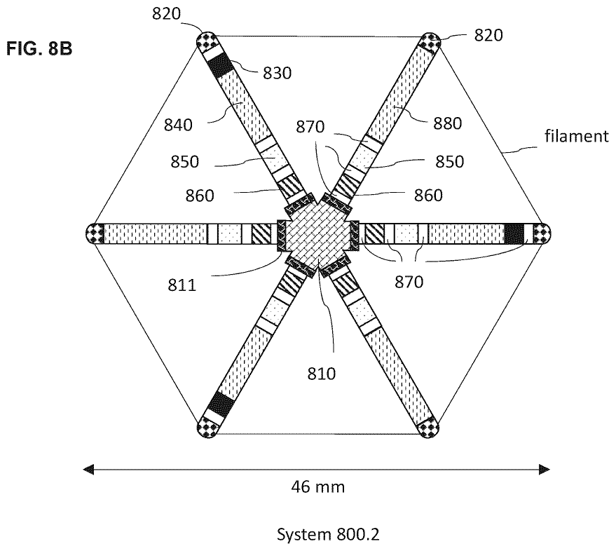

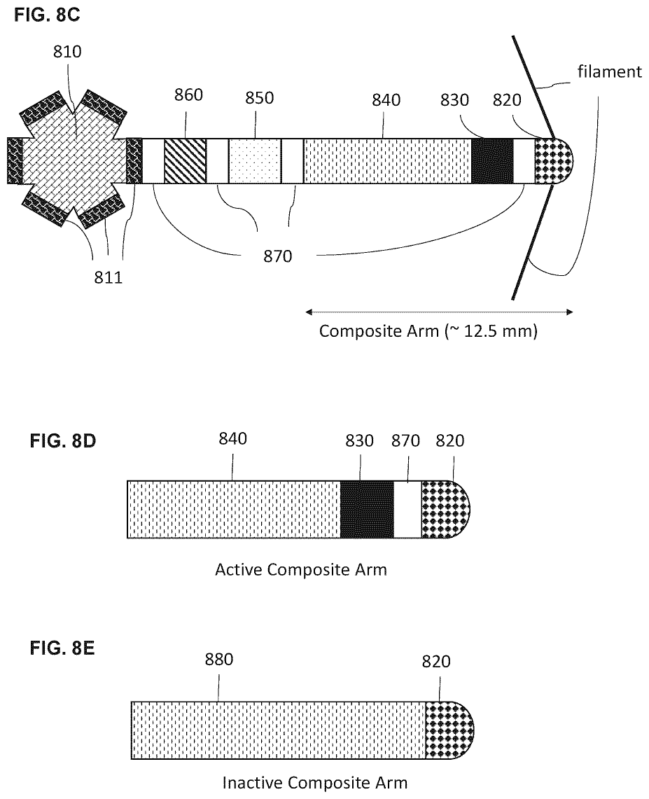

FIG. 8A and FIG. 8B are labelled to show the various elements of two such configurations. A shown in FIGS. 8A and 8B, the system 800.1 and 800.2 each comprises a central elastomeric core 810 which is in the shape of an “asterisk” having six short branches. FIG. 8C displays one arm attached to the central elastomeric core 810. Segment 811 is an inert polycaprolactone linker segment at the end of each branch of the core which facilitates the attachment of the arms.

For an arm containing a drug-eluting segment, a segment 870 of the arm is attached to one short asterisk branch. The segment 870 is followed by a segment 860, a second segment 870, a segment 850, a third segment 870, a segment 840, a segment 830, and a forth segment 870 in sequence. The distal end of each drug-containing arm has segment 820. FIG. 8D shows an active composite arm (distal end of drug-eluting arm), comprising a segment 840, a segment 830, a forth segment 870, and a distal segment 820 in sequence. As shown in FIGS. 8C and 8D, the active composite arm can measure about 14.0 mm, and can be trimmed to about 12.5 mm.

For an arm not containing a drug-eluting segment, a segment 870 of the arm is attached to one short asterisk branch. The segment 870 is followed by a segment 860, a second segment 870, a segment 850, a third segment 870, and a segment 880. The distal end of each drug-free arm has segment 820. FIG. 8E shows an inactive composite arm (distal end of non-drug-eluting arm), comprising a segment 880, and a distal segment 820 in sequence. The inactive composite arm can measure about 14.0 mm, and can be trimmed to about 12.5 mm.

The gastric residence system has an average size of about 46 mm and each segment has a length ranging from about 0.5 mm to about 8.5 mm. Table B below provides a listing of the length of each segment in the gastric residence system. Each range or value below can be considered to be “about” the range or value indicated, or exactly the range or value indicated.

TABLE B

| Segment | Length | |
|---|---|---|
| 820 | 4 | mm |
| 830 | 2.4 | mm |
| 840 | 5.6 | mm |
| 850 | 1.85 | mm |
| 860 | 1.0 | mm |
| 870 | 0.5 | mm |
| 880 | 8.5 | mm |

In the gastric residence system, the cross-section of an arm could be an oval, a circle, a rectangle, a square, or a triangle. In one exemplary system, the cross-section of an arm could be an equilateral triangle, wherein the side of the triangle is about 3.0 mm-3.5 mm, such as about 3.3 mm. In one exemplary system, the cross-section of an arm could be a square or a rectangle, wherein the diagonal of the square or rectangle is about 3.0 mm-3.5 mm, such as about 3.3 mm. In one exemplary system, the cross-section of an arm could be a circle, wherein the diameter of the circle is about 3.0 mm-3.5 mm, such as about 3.3 mm.

The central elastomeric core 810 comprises a liquid silicone rubber (LSR) having a hardness of 50 durometer.

In one example, the dosage form provided here contains 6 arms each comprising a drug-eluting segment, wherein the dosage form comprises about 28 mg of risperidone for administration (FIG. 8A). In one example, the dosage form provided here contains 3 arms each comprising a drug-eluting segment, wherein the dosage form comprises about 14 mg of risperidone for administration (FIG. 8B). Risperidone is included in a carrier polymer-agent segment 830 (e.g., a drug-eluting segment). The drug-eluting segment comprises about 35.0 wt % of risperidone, about 55.9 wt % of Corbion PC17, about 5.0 wt % of VA64, about 3.0 wt % of P407, about 0.5 wt % of Vitamin E succinate, about 0.5 wt % of SiO₂, and about 0.1 wt % of pigment. The pigment includes about 0.05% of FD&C Yellow 5 Alum lake (14-16%) and about 0.05% of FD&C Blue 1 Alum lake (11-13%). Also contemplated in the present application are variations of this dosage form with increased numbers and/or lengths of the drug-eluting segments to achieve higher doses of the drug, for example, risperidone.

Moreover, each arm comprises inactive segment (segment 840 for drug-containing arms and segment 880 for drug-free arms). The inactive segment 840 or 880 each comprises about 66.45 wt % of Corbion PC17, about 32.0 wt % of VA 64, about 1.5 wt % of P407 and about 0.05 wt % of FD&C Blue 1 Aluminum lake.

The gastric residence system further includes a time-dependent disintegrating matrix or linker, referred as the segment 860, as well as a pH-dependent disintegrating matrix or linker, referred as the segment 850. In addition, the gastric residence system includes a structural segment 870.

The time-dependent disintegrating matrix (segment 860) comprises about 44.95 wt % Corbion PC17, about 35 wt % of acid terminated copolymer of DL-lactide and glycolide (PDLG5004A), about 18 wt % of copolymer of DL-lactide and glycolide (PDLG5004), about 2 wt % of polyethylene glycol 100 k and about 0.05 wt % color-absorbing dye E172. The pH-dependent disintegrating matrix (segment 850) comprises about 63.95 wt % HPMCAS, about 33.95 wt % Corbion PC17, about 2 wt % P407 and about 0.1 wt % color-absorbing dye E172. The structural segment 870 can be a radiopaque-PCL segment, comprising about 70 wt % PCL, and about 30 wt % (BiO)₂CO₃.

Segment 820, at the distal end of each arm, is a third disintegrating matrix, to which a filament is also optionally attached, where the filament thereby circumferentially connects the arms. The third disintegrating matrix (Segment 820) comprises about 64.9 wt % HPMCAS, about 30 wt % PCL, about 2.5 wt % propylene glycol, about 2.5 wt % stearic acid, and about 0.1 wt % iron oxide (for example about 0.025% ferrosoferric oxide and about 0.075% FD&C Red 40). The filament can comprise materials of about Shore hardness 70 A to 85 A, such as polyurethane of hardness 80 A. The filament can comprise polyurethane, such as Pellethane 80 A. Each section of filament connecting two adjacent arms can be about 20 to about 25 mm in length, such as about 21 to about 24 mm in length. The circumferential filament can be about 105 mm in total length.

In the gastric residence system, each drug arm is coated by a release rate-modulating film. Specifically, the coating comprises about 73.5 wt % of Corbion PC17, about 24.5 wt % of VA64, and about 2.0 wt % of Mg stearate, and is applied in an amount of about 2.3% to about 3%, such as about 2.6% of the pre-coating weight of the segment, (i.e., segments, 830, 840 and 880).

The gastric residence system is assembled and then placed into an appropriate sized capsule as described in Example 1 of International Patent Application PCT/US2020/059541. The dosage form described here differs from a gastric residence system previously described in International Patent Application No. PCT/US2020/059541, and other gastric residence systems previously designated as LYN-005.

In another example of the risperidone-formulated gastric residence system, the stellate contains 4 arms each comprising a drug-eluting segment, and 2 arms not comprising a drug-eluting segment. In another example of the risperidone-formulated gastric residence system, the stellate contains 2 arms each comprising a drug-eluting segment, and 4 arms not comprising a drug-eluting segment. Also contemplated in this application are other gastric residence systems containing 6 arms of which either 1, 2, 3, 4, 5, or 6 arms comprise a drug-eluting segment.

The described gastric residence systems, while shown as being risperidone-formulated, is not limited as such, and can be used with other drugs by replacing the segment(s) containing risperidone and/or replacing inert segment(s), with segments containing other drugs.

### Example 5: Risperidone Dosage Form (Extended-Release Gastric Residence System)

In this Example, a dosage form according to the present invention includes a gastric residence system, the gastric residence system is formulated to include risperidone.

The gastric residence system includes a central elastomer that provides the gastric residence system with the ability to be compacted into a compressed configuration. The gastric residence system illustrated in this Example is another different arrangement of the “star” configuration. In an example of the risperidone-formulated gastric residence system, the stellate contains 6 arms each comprising a drug-eluting segment.

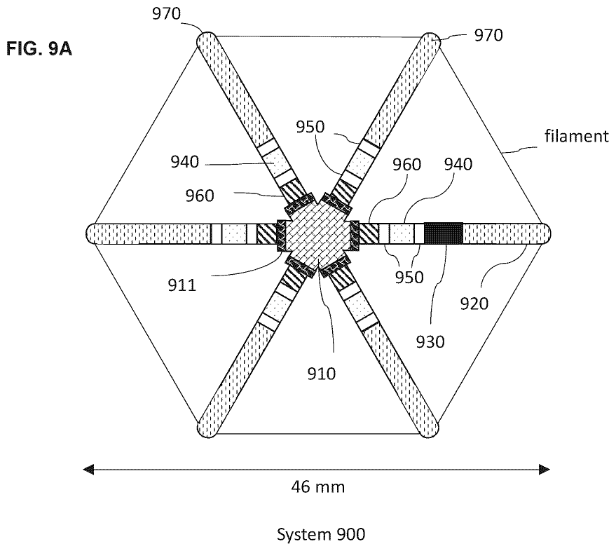

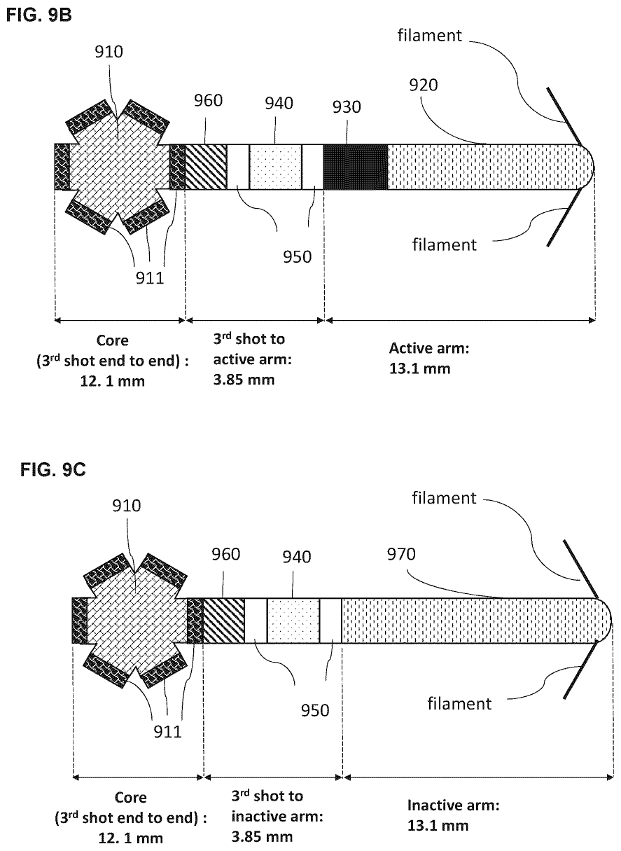

FIG. 9A is labelled to show the various elements of this configuration. The system 900 comprises a central elastomeric core 910 which is in the shape of an “asterisk” having six short branches. Segment 911 is an inert polycaprolactone linker segment (“3^{rd} shot”) at the end of each branch of the core which facilitates the attachment of the arms.

For an arm containing a drug-eluting segment, a segment 960 of the arm is attached to one short asterisk branch. The segment 960 is followed by a first segment 950, a segment 940, a second segment 950, and a segment 930 in sequence. The distal end of each drug-containing arm has segment 920. An exemplary drug-eluting arm is illustrated in FIG. 9B.

For an arm not containing a drug-eluting segment, a segment 960 of the arm is attached to one short asterisk branch. The segment 960 is followed by a first segment 950, a segment 940, and a second segment 950 in sequence. The distal end of each drug-free arm has segment 970. An exemplary non-drug-eluting arm is illustrated in FIG. 9C.

The gastric residence system has an average size of about 46 mm and each segment has a length ranging from about 0.5 mm to about 13.1 mm. Table C below provides a listing of the length of each segment in the gastric residence system. Each range or value below can be considered to be “about” the range or value indicated, or exactly the range or value indicated.

TABLE C

| Segment | Length | |
|---|---|---|
| 920 | 5.3 | mm |
| 930 | 7.8 | mm |
| 940 | 1.85 | mm |
| 950 | 0.5 | mm |
| 960 | 1.0 | mm |
| 970 | 13.1 | mm |

In the gastric residence system, the cross-section of an arm could be an oval, a circle, a rectangle, a square, or a triangle. In one exemplary system, the cross-section of an arm could be an equilateral triangle, wherein the side of the triangle is about 3.0 mm-3.5 mm, such as about 3.3 mm. In one exemplary system, the cross-section of an arm could be a square or a rectangle, wherein the diagonal of the square or rectangle is about 3.0 mm-3.5 mm, such as about 3.3 mm. In one exemplary system, the cross-section of an arm could be a circle, wherein the diameter of the circle is about 3.0 mm-3.5 mm, such as about 3.3 mm.

The central elastomeric core 910 comprises a liquid silicone rubber (LSR) having a hardness of 50 durometer.

In this example, the dosage form provided here contains 6 arms, one of which comprises a drug-eluting segment, wherein the dosage form comprises about 16 mg of risperidone for administration. Risperidone is included in a carrier polymer-agent segment 930 (e.g., a drug-eluting segment). The drug-eluting segment comprises about 35.0 wt % of risperidone, about 55.9 wt % of Corbion PC17, about 5.0 wt % of VA64, about 3.0 wt % of P407, about 0.5 wt % of Vitamin E succinate, about 0.5 wt % of SiO₂, and about 0.1 wt % of pigment. The pigment includes about 0.05% of FD&C Yellow 5 Alum lake (16-18%) and about 0.05% of FD&C Blue 1 Alum lake (11-13%). Also contemplated in the present application are variations of this dosage form with increased numbers and/or lengths of the drug-eluting segments to achieve higher doses of the drug, for example, risperidone.

The gastric residence system further includes a time-dependent disintegrating matrix or linker, referred as the segment 960, as well as a pH-dependent disintegrating matrix or linker, referred as the segment 940. In addition, the gastric residence system includes a structural segment 950.

The time-dependent disintegrating matrix (segment 960) comprises about 44.95 wt % Corbion PC12, about 35 wt % of acid terminated copolymer of DL-lactide and glycolide (PDLG5004A), about 18 wt % of copolymer of DL-lactide and glycolide (PDLG5004), about 2 wt % of polyethylene glycol 100 k and about 0.05 wt % color-absorbing dye E172. The pH-dependent disintegrating matrix (segment 940) comprises about 64.0 wt % HPMCAS, about 34.0 wt % Corbion PC17, and about 2 wt % P407. The structural segment 950 can be a radiopaque-PCL segment, comprising about 70 wt % PCL, and about 30 wt % (BiO)₂CO₃.

Each arm comprises an inactive segment (segment 920 for drug-containing arms and segment 970 for drug-free arms) at the distal end of the arm, to which a filament is also attached, where the filament thereby circumferentially connects the arms. The inactive segment 920 or 970 each comprises about 66.45 wt % of Corbion PC17, about 32.0 wt % of VA 64, about 1.5 wt % of P407 and about 0.05 wt % of FD&C Blue 1 Aluminum lake. The filament can be a disintegrating filament and can comprise poly (lactic-co-glycolic acid) and/or polyglycolic acid. Each section of filament connecting two adjacent arms can be about 20 to about 25 mm in length, such as about 21 to about 24 mm in length. In one exemplary system, the filament is Bondek Suture 2-0 or Bondek Suture 3-0.

In the gastric residence system, each drug arm is coated by a release rate-modulating film. Specifically, the coating comprises about 73.5 wt % of Corbion PC17, about 24.5 wt % of VA64, and about 2.0 wt % of Mg stearate, and is applied in an amount of about 2.5% to about 3.2%, such as about 2.8% of the pre-coating weight of the segment (i.e., segments 920, 930, 970).

The gastric residence system is assembled and then placed into an appropriate sized capsule as described in Example 1 of International Patent Application PCT/US2020/059541. The dosage form described here differs from a gastric residence system previously described in International Patent Application No. PCT/US2020/059541, and other gastric residence systems previously designated as LYN-005.

In another example of the risperidone-formulated gastric residence system, the stellate contains 3 arms each comprising a drug-eluting segment, and 3 arms not comprising a drug-eluting segment. In another example of the risperidone-formulated gastric residence system, the stellate contains 2 arms each comprising a drug-eluting segment, and 4 arms not comprising a drug-eluting segment. Also contemplated in this application are other gastric residence systems containing 6 arms of which either 1, 2, 3, 4, 5, or 6 arms comprise a drug-eluting segment. In an example wherein 2 of the arms comprise a drug-eluting segment, the dosage form comprises about 32 mg of risperidone for administration. In an example wherein 3 of the arms comprise a drug-eluting segment, the dosage form comprises about 48 mg of risperidone for administration.

The described gastric residence systems, while shown as being risperidone-formulated, is not limited as such, and can be used with other drugs by replacing the segment(s) containing risperidone and/or replacing inert segment(s), with segments containing other drugs.

### Example 6: Risperidone Dosage Form (Extended-Release Gastric Residence System)

In this Example, a dosage form according to the present invention includes a gastric residence system, the gastric residence system is formulated to include risperidone.

The gastric residence system includes a central elastomer that provides the gastric residence system with the ability to be compacted into a compressed configuration. The gastric residence system illustrated in this Example is another different arrangement of the “star” configuration. In an example of the risperidone-formulated gastric residence system, the stellate contains 6 arms each comprising a drug-eluting segment.

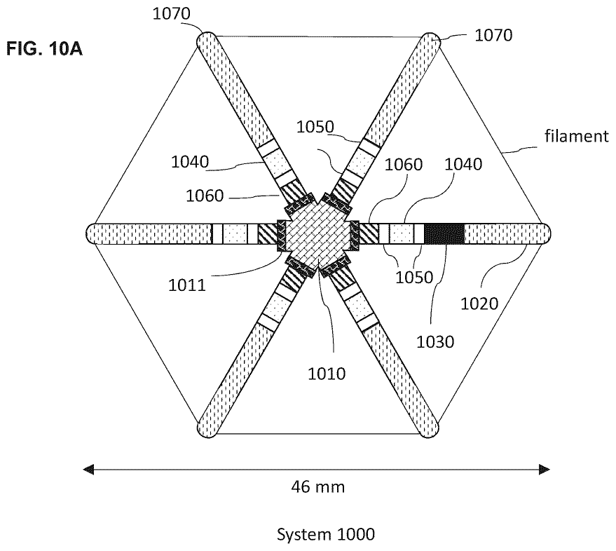

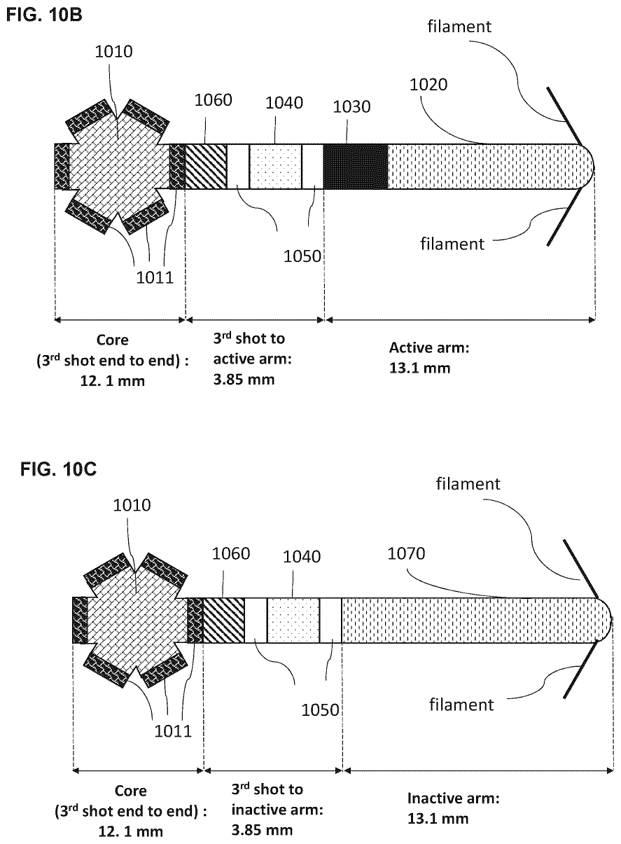

FIG. 10A is labelled to show the various elements of this configuration. The system 1000 comprises a central elastomeric core 1010 which is in the shape of an “asterisk” having six short branches. Segment 1011 is an inert polycaprolactone linker segment (“3^{rd} shot”) at the end of each branch of the core which facilitates the attachment of the arms.

For an arm containing a drug-eluting segment, a segment 1060 of the arm is attached to one short asterisk branch. The segment 1060 is followed by a first segment 1050, a segment 1040, a second segment 1050, and a segment 1030 in sequence. The distal end of each drug-containing arm has segment 1020. An exemplary drug-eluting arm is illustrated in FIG. 10B.

For an arm not containing a drug-eluting segment, a segment 1060 of the arm is attached to one short asterisk branch. The segment 1060 is followed by a first segment 1050, a segment 1040, and a second segment 1050 in sequence. The distal end of each drug-free arm has segment 1070. An exemplary non-drug-eluting arm is illustrated in FIG. 10C.

The gastric residence system has an average size of about 46 mm and each segment has a length ranging from about 0.5 mm to about 13.1 mm. Table D below provides a listing of the length of each segment in the gastric residence system. Each range or value below can be considered to be “about” the range or value indicated, or exactly the range or value indicated.

TABLE D

| Segment | Length | |
|---|---|---|
| 1020 | 5.3 | mm |
| 1030 | 7.8 | mm |
| 1040 | 1.85 | mm |
| 1050 | 0.5 | mm |
| 1060 | 1.0 | mm |
| 1070 | 13.1 | mm |

In the gastric residence system, the cross-section of an arm could be an oval, a circle, a rectangle, a square, or a triangle. In one exemplary system, the cross-section of an arm could be an equilateral triangle, wherein the side of the triangle is about 3.0 mm-3.5 mm, such as about 3.3 mm. In one exemplary system, the cross-section of an arm could be a square or a rectangle, wherein the diagonal of the square or rectangle is about 3.0 mm-3.5 mm, such as about 3.3 mm. In one exemplary system, the cross-section of an arm could be a circle, wherein the diameter of the circle is about 3.0 mm-3.5 mm, such as about 3.3 mm.

The central elastomeric core 1010 comprises a liquid silicone rubber (LSR) having a hardness of 50 durometer.

In this example, the dosage form provided here contains 6 arms, one of which comprises a drug-eluting segment, wherein the dosage form comprises about 16 mg of risperidone for administration. Risperidone is included in a carrier polymer-agent segment 1030 (e.g., a drug-eluting segment). The drug-eluting segment comprises about 35.0 wt % of risperidone, about 55.9 wt % of Corbion PC17, about 5.0 wt % of VA64, about 3.0 wt % of P407, about 0.5 wt % of Vitamin E succinate, about 0.5 wt % of SiO₂, and about 0.1 wt % of pigment. The pigment includes about 0.05% of FD&C Yellow 5 Alum lake (16-18%) and about 0.05% of FD&C Blue 1 Alum lake (11-13%). Also contemplated in the present application are variations of this dosage form with increased numbers and/or lengths of the drug-eluting segments to achieve higher doses of the drug, for example, risperidone.

The gastric residence system further includes a time-dependent disintegrating matrix or linker, referred as the segment 1060, as well as a pH-dependent disintegrating matrix or linker, referred as the segment 1040. In addition, the gastric residence system includes a structural segment 1050.

The time-dependent disintegrating matrix (segment 1060) comprises about 49.95 wt % Corbion PC12, about 32 wt % of acid terminated copolymer of DL-lactide and glycolide (PDLG5004A), about 16 wt % of copolymer of DL-lactide and glycolide (PDLG5004), about 2 wt % of polyethylene glycol 100 k and about 0.05 wt % color-absorbing dye E172. The pH-dependent disintegrating matrix (segment 1040) comprises about 64.0 wt % HPMCAS, about 34.0 wt % Corbion PC17, and about 2 wt % P407. The structural segment 1050 can be a radiopaque-PCL segment, comprising about 70 wt % PCL, and about 30 wt % (BiO)₂CO₃.

Each arm comprises an inactive segment (segment 1020 for drug-containing arms and segment 1070 for drug-free arms) at the distal end of the arm, to which a filament is also attached, where the filament thereby circumferentially connects the arms. The inactive segment 1020 or 1070 each comprises about 66.45 wt % of Corbion PC17, about 32.0 wt % of VA 64, about 1.5 wt % of P407 and about 0.05 wt % of FD&C Blue 1 Aluminum lake. The filament can be a disintegrating filament and can comprise poly (lactic-co-glycolic acid) and/or polyglycolic acid. Each section of filament connecting two adjacent arms can be about 20 to about 25 mm in length, such as about 21 to about 24 mm in length. In one exemplary system, the filament is Bondek Suture 2-0 or Bondek Suture 3-0.

In the gastric residence system, each drug arm is coated by a release rate-modulating film. Specifically, the coating comprises about 73.5 wt % of Corbion PC17, about 24.5 wt % of VA64, and about 2.0 wt % of Mg stearate, and is applied in an amount of about 2.5% to about 3.2%, such as about 2.8% of the pre-coating weight of the segment (i.e., segments 1020, 1030, 1070).

The gastric residence system is assembled and then placed into an appropriate sized capsule as described in Example 1 of International Patent Application PCT/US2020/059541. The dosage form described here differs from a gastric residence system previously described in International Patent Application No. PCT/US2020/059541, and other gastric residence systems previously designated as LYN-005.

In another example of the risperidone-formulated gastric residence system, the stellate contains 3 arms each comprising a drug-eluting segment, and 3 arms not comprising a drug-eluting segment. In another example of the risperidone-formulated gastric residence system, the stellate contains 2 arms each comprising a drug-eluting segment, and 4 arms not comprising a drug-eluting segment. Also contemplated in this application are other gastric residence systems containing 6 arms of which either 1, 2, 3, 4, 5, or 6 arms comprise a drug-eluting segment. In an example wherein 2 of the arms comprise a drug-eluting segment, the dosage form comprises about 32 mg of risperidone for administration. In an example wherein 3 of the arms comprise a drug-eluting segment, the dosage form comprises about 48 mg of risperidone for administration.

The described gastric residence systems, while shown as being risperidone-formulated, is not limited as such, and can be used with other drugs by replacing the segment(s) containing risperidone and/or replacing inert segment(s), with segments containing other drugs.

### Example 7: Risperidone Dosage Form (Extended-Release Gastric Residence System)

In this Example, a dosage form according to the present invention includes a gastric residence system, the gastric residence system is formulated to include risperidone.

The gastric residence system includes a central elastomer that provides the gastric residence system with the ability to be compacted into a compressed configuration. The gastric residence system illustrated in this Example is another different arrangement of the “star” configuration. In an example of the risperidone-formulated gastric residence system, the stellate contains 6 arms each comprising a drug-eluting segment.

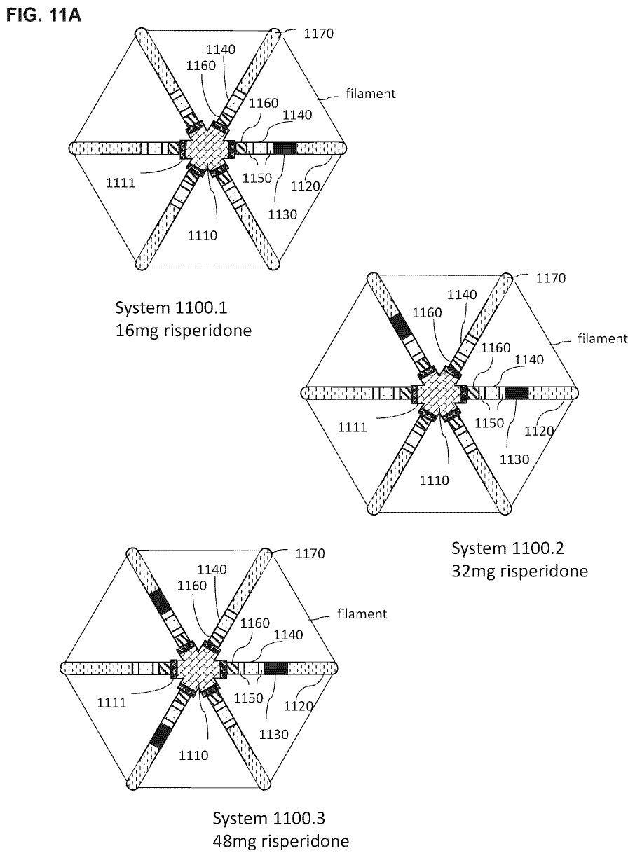

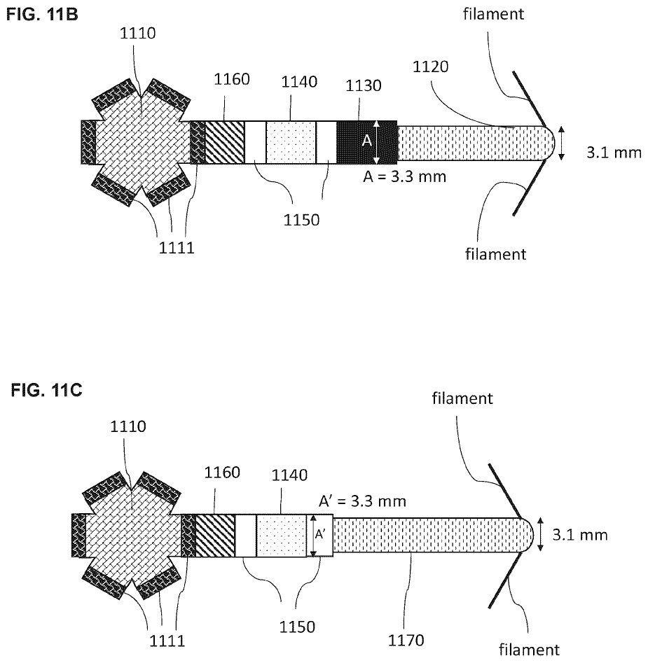

FIG. 11A is labelled to show the various elements of such configurations. Each of the system 1100.1, 1100. 2 and 1100.3 comprises a central elastomeric core 1110 which is in the shape of an “asterisk” having six short branches. Segment 1111 is an inert polycaprolactone linker segment (“3^{rd} shot”) at the end of each branch of the core which facilitates the attachment of the arms.

For an arm containing a drug-eluting segment, a segment 1160 of the arm is attached to one short asterisk branch. The segment 1160 is followed by a first segment 1150, a segment 1140, a second segment 1150, and a segment 1130 in sequence. The distal end of each drug-containing arm has segment 1120. An exemplary drug-eluting arm is illustrated in FIG. 11B.

For an arm not containing a drug-eluting segment, a segment 1160 of the arm is attached to one short asterisk branch. The segment 1160 is followed by a first segment 1150, a segment 1140, and a second segment 1150 in sequence. The distal end of each drug-free arm has segment 1170. An exemplary non-drug-eluting arm is illustrated in FIG. 11C.

The gastric residence system has an average size of about 46 mm and each segment has a length ranging from about 0.5 mm to about 13.1 mm. Table E below provides a listing of the length of each segment in the gastric residence system. Each range or value below can be considered to be “about” the range or value indicated, or exactly the range or value indicated.

TABLE E

| Segment | Length | |
|---|---|---|
| 1120 | 5.3 | mm |
| 1130 | 7.8 | mm |
| 1140 | 1.85 | mm |
| 1150 | 0.5 | mm |
| 1160 | 1.0 | mm |
| 1170 | 13.1 | mm |

In the gastric residence system, the cross-section of an arm could be an oval, a circle, a rectangle, a square, or a triangle. In one exemplary system, the cross-section of an arm could be an equilateral triangle, wherein the side of the triangle is about 3.0 mm-3.5 mm, such as about 3.3 mm. In one exemplary system, the cross-section of an arm could be a square or a rectangle, wherein the diagonal of the square or rectangle is about 3.0 mm-3.5 mm, such as about 3.3 mm. In one exemplary system, the cross-section of an arm could be a circle, wherein the diameter of the circle is about 3.0 mm-3.5 mm, such as about 3.3 mm.

In one exemplary gastric residence system, the cross-section of an arm in each of segments 1130, 1140, 1150 and 1160 is an equilateral triangle, wherein the side of the equilateral triangle measures 3.3 mm. In an exemplary gastric residence system, the cross section of an arm in segment 1120 is an equilateral triangle, wherein the side of the equilateral triangle measures 3.1 mm. In an exemplary gastric residence system, the cross section of an arm in segment 1170 is an equilateral triangle, wherein the side of the equilateral triangle measures 3.1 mm.

Table F below provides a listing of the length of each segment in the gastric residence system, as well as thickness of the segment (measured from dimensions of the segment cross section). Each range or value below can be considered to be “about” the range or value indicated, or exactly the range or value indicated.

TABLE F

| | | |
|---|---|---|
| Segment | Length | Thickness |

| | | | |
|---|---|---|---|
| 1120 | 5.3 | mm | 3.1 mm |
| 1130 | 7.8 | mm | 3.3 mm |
| 1140 | 1.85 | mm | 3.3 mm |
| 1150 | 0.5 | mm | 3.3 mm |
| 1160 | 1.0 | mm | 3.3 mm |
| 1170 | 13.1 | mm | 3.1 mm |

In some embodiments according to any of the systems described herein, the thickness of a segment is determined by the longest straight line within a cross-section in the segment. In some embodiments wherein the cross-section of the segment is a circle, the thickness is defined by the diameter of the circle. In some embodiments, wherein the cross-section of the segment is a square or rectangle, the thickness is defined by the diagonal of the square or rectangle. In some embodiments, wherein the cross-section of the segment is an equilateral triangle, the thickness is defined by the side of the equilateral triangle.

In an exemplary gastric residence system shown above, the segments, the cross-sections of the segments are equilateral triangles, wherein the thickness is defined by the side of the equilateral triangle.

The central elastomeric core 1110 comprises a liquid silicone rubber (LSR) having a hardness of 50 durometer.

In this example, the dosage form provided here contains 6 arms, one of which comprises a drug-eluting segment, wherein the dosage form comprises about 16 mg of risperidone for administration. Risperidone is included in a carrier polymer-agent segment 1130 (e.g., a drug-eluting segment). The drug-eluting segment comprises about 35.0 wt % of risperidone, about 55.9 wt % of Corbion PC17, about 5.0 wt % of VA64, about 3.0 wt % of P407, about 0.5 wt % of Vitamin E succinate, about 0.5 wt % of SiO₂, and about 0.1 wt % of pigment. The pigment includes about 0.05% of FD&C Yellow 5 Alum lake (16-18%) and about 0.05% of FD&C Blue 1 Alum lake (11-13%). Also contemplated in the present application are variations of this dosage form with increased numbers and/or lengths of the drug-eluting segments to achieve higher doses of the drug, for example, risperidone.

The gastric residence system further includes a time-dependent disintegrating matrix or linker, referred as the segment 1160, as well as a pH-dependent disintegrating matrix or linker, referred as the segment 1140. In addition, the gastric residence system includes a structural segment 1150.

The time-dependent disintegrating matrix (segment 1160) comprises either: (a) about 44.95 wt % Corbion PC12, about 35 wt % of acid terminated copolymer of DL-lactide and glycolide (PDLG5004A), about 18 wt % of copolymer of DL-lactide and glycolide (PDLG5004), about 2 wt % of polyethylene glycol 100 k and about 0.05 wt % color-absorbing dye E172; or (b): about 49.95 wt % Corbion PC12, about 32 wt % of acid terminated copolymer of DL-lactide and glycolide (PDLG5004A), about 16 wt % of copolymer of DL-lactide and glycolide (PDLG5004), about 2 wt % of polyethylene glycol 100 k and about 0.05 wt % color-absorbing dye E172. The pH-dependent disintegrating matrix (segment 1140) comprises about 64.0 wt % HPMCAS, about 34.0 wt % Corbion PC17, and about 2 wt % P407. The structural segment 1150 can be a radiopaque-PCL segment, comprising about 70 wt % PCL, and about 30 wt % (BiO)₂CO₃.

Each arm comprises an inactive segment (segment 1120 for drug-containing arms and segment 1170 for drug-free arms) at the distal end of the arm, to which a filament is also attached, where the filament thereby circumferentially connects the arms. The inactive segment 1120 or 1170 each comprises about 66.45 wt % of Corbion PC17, about 32.0 wt % of VA 64, about 1.5 wt % of P407 and about 0.05 wt % of FD&C Blue 1 Aluminum lake. The filament can be a disintegrating filament and can comprise poly (lactic-co-glycolic acid) and/or polyglycolic acid. Each section of filament connecting two adjacent arms can be about 20 to about 25 mm in length, such as about 21 to about 24 mm in length. In one exemplary system, the filament is Bondek Suture 2-0 or Bondek Suture 3-0.

In the gastric residence system, each drug arm is coated by a release rate-modulating film. Specifically, the coating comprises about 73.5 wt % of Corbion PC17, about 24.5 wt % of VA64, and about 2.0 wt % of Mg stearate, and is applied in an amount of about 2.5% to about 3.2%, such as about 2.8% of the pre-coating weight of the segment (i.e., segments 1120, 1130, 1170).

The gastric residence system is assembled and then placed into an appropriate sized capsule as described in Example 1 of International Patent Application PCT/US2020/059541. The dosage form described here differs from a gastric residence system previously described in International Patent Application No. PCT/US2020/059541, and other gastric residence systems previously designated as LYN-005.

In another example of the risperidone-formulated gastric residence system, the stellate contains 3 arms each comprising a drug-eluting segment, and 3 arms not comprising a drug-eluting segment. In another example of the risperidone-formulated gastric residence system, the stellate contains 2 arms each comprising a drug-eluting segment, and 4 arms not comprising a drug-eluting segment. Also contemplated in this application are other gastric residence systems containing 6 arms of which either 1, 2, 3, 4, 5, or 6 arms comprise a drug-eluting segment. In an example wherein 1 of the arms comprise a drug-eluting segment, the dosage form comprises about 16 mg of risperidone for administration (FIG. 11A top). In an example wherein 3 of the arms comprise a drug-eluting segment, the dosage form comprises about 48 mg of risperidone for administration. In an example wherein 2 of the arms comprise a drug-eluting segment, the dosage form comprises about 32 mg of risperidone for administration. (FIG. 11A center) In an example wherein 3 of the arms comprise a drug-eluting segment, the dosage form comprises about 48 mg of risperidone for administration (FIG. 11A bottom).

The described gastric residence systems, while shown as being risperidone-formulated, is not limited as such, and can be used with other drugs by replacing the segment(s) containing risperidone and/or replacing inert segment(s), with segments containing other drugs.

The disclosures of all publications, patents, patent applications and published patent applications referred to herein by an identifying citation are hereby incorporated herein by reference in their entirety. Web sites referenced using “World-Wide-Web” at the beginning of the Uniform Resource Locator (URL) can be accessed by replacing “World-Wide-Web” with “www.”

Although the foregoing invention has been described in some detail by way of illustration and example for purposes of clarity of understanding, it is apparent to those skilled in the art that certain changes and modifications will be practiced. Therefore, the description and examples should not be construed as limiting the scope of the invention.

1. A gastric residence system comprising:
six arms affixed to a central elastomer, wherein at least one arm comprises a drug-eluting segment;
each arm comprising a proximal end, a distal end, and an outer surface therebetween; wherein the proximal end of each arm is attached to the central elastomer and projects radially from the central elastomer, each arm having its distal end not attached to the central elastomer component and located at a larger radial distance from the central elastomer component than the proximal end;
wherein the at least one arm comprising a drug eluting segment comprises:
a first inert segment;
a first disintegrating matrix segment attached to the first inert segment;
a second inert segment attached to the first disintegrating matrix segment;
a second disintegrating matrix segment attached to the second inert segment;
a third inert segment attached to the second disintegrating matrix segment;
a fourth inert segment attached to the third inert segment;
the drug eluting segment attached to the fourth inert segment, wherein the drug eluting segment comprises a carrier polymer, and risperidone or a salt thereof, and wherein the drug eluting segment further comprises a coating comprising a release rate-modulating polymer film;
an optional fifth inert segment attached to the drug eluting segment; and
a third disintegrating matrix segment which is attached to the optional fifth inert segment when the optional fifth inert segment is present, or which is attached to the drug eluting segment when the optional fifth inert segment is not present;
and
a filament circumferentially connecting each arm.

2. A gastric residence system comprising:
six arms affixed to a central elastomer, wherein at least one arm comprises a drug-eluting segment;
each arm comprising a proximal end, a distal end, and an outer surface therebetween; wherein the proximal end of each arm is attached to the elastomer component and projects radially from the elastomer component, each arm having its distal end not attached to the elastomer component and located at a larger radial distance from the elastomer component than the proximal end;
wherein the at least one arm comprising a drug eluting segment comprises:
a first inert segment;
a first disintegrating matrix segment attached to the first inert segment;
a second inert segment attached to the first disintegrating matrix segment;
a second disintegrating matrix segment attached to the second inert segment;
a third inert segment attached to the second disintegrating matrix segment;
a fourth inert segment attached to the third inert segment;
the drug eluting segment attached to the fourth inert segment, wherein the drug eluting segment comprises a carrier polymer, and risperidone or a salt thereof, and wherein the drug eluting segment further comprises a coating comprising a release rate-modulating polymer film;
an optional fifth inert segment attached to the drug eluting segment;
a third disintegrating matrix segment attached to the optional fifth inert segment;
and
a filament circumferentially connecting each arm.

3-9. (canceled)

10. A gastric residence system comprising:
six arms affixed to a central elastomer, wherein at least one arm comprises a drug-eluting segment;
each arm comprising a proximal end, a distal end, and an outer surface therebetween; wherein the proximal end of each arm is attached to the elastomer component and projects radially from the elastomer component, each arm having its distal end not attached to the elastomer component and located at a larger radial distance from the elastomer component than the proximal end;
wherein the at least one arm comprising a drug eluting segment comprises:
a first inert segment;
a first disintegrating matrix segment attached to the first inert segment;
a second inert segment attached to the first disintegrating matrix segment;
a second disintegrating matrix segment attached to the second inert segment;
a third inert segment attached to the second disintegrating matrix segment;
a fourth inert segment attached to the third inert segment;
the drug eluting segment attached to the fourth inert segment, wherein the drug eluting segment comprises a carrier polymer, and risperidone or a salt thereof, and wherein the drug eluting segment further comprises a coating comprising a release rate-modulating polymer film;
a fifth inert segment attached to the drug eluting segment;
and
an optional filament circumferentially connecting each arm.

11-30. (canceled)

31. A gastric residence system comprising:
six arms affixed to a central elastomer, wherein at least one arm comprises a drug-eluting segment;
each arm comprising a proximal end, a distal end, and an outer surface therebetween; wherein the proximal end of each arm is attached to the central elastomer and projects radially from the central elastomer, each arm having its distal end not attached to the central elastomer and located at a larger radial distance from the central elastomer than the proximal end;
wherein the at least one arm comprising a drug eluting segment comprises:
a first disintegrating matrix segment;
a first inert segment attached to the first disintegrating matrix segment;
a second disintegrating matrix segment attached to the first inert segment;
a second inert segment attached to the second disintegrating matrix segment;
the drug eluting segment attached to the second inert segment, wherein the drug eluting segment comprises a carrier polymer, and risperidone or a salt thereof, and wherein the drug eluting segment further comprises a coating comprising a release rate-modulating polymer film;
a third inert segment attached to the drug eluting segment;
and
a filament circumferentially connecting each arm.

32. (canceled)

33. The gastric residence system of claim 31, wherein the segments are in the order listed from the proximal end to the distal end of the arm comprising a drug eluting segment, where the first disintegrating matrix segment is at the proximal end of the arm comprising the drug eluting segment, the first disintegrating matrix segment is attached to the central elastomer, and the third inert segment is at the distal end of the arm comprising a drug eluting segment.

34. The gastric residence system of claim 31, wherein at least one arm excludes a drug eluting segment.

35. The gastric residence system of claim 34, wherein the at least one arm excluding a drug eluting segment comprises:
a first disintegrating matrix segment;
a first inert segment attached to the first disintegrating matrix segment;
a second disintegrating matrix segment attached to the first inert segment;
a second inert segment attached to the second disintegrating matrix segment;
a third inert segment attached to the second inert segment;
and
a filament circumferentially connecting each arm.

36. (canceled)

37. The gastric residence system of claim 35, wherein the segments are in the order listed from the proximal end to the distal end of the arm excluding a drug eluting segment, where the first disintegrating matrix segment is at the proximal end of the arm excluding a drug eluting segment, the first disintegrating matrix segment is attached to the central elastomer, and the third inert segment is at the distal end of the arm excluding drug eluting segment.

38. The gastric residence system of claim 31, wherein:
(a) one arm comprises the drug eluting segment and five arms exclude the drug eluting segment; or
(b) two arms comprise the drug eluting segment and four arms exclude the drug eluting segment; or
(c) three arms comprise the drug eluting segment and three arms exclude the drug eluting segment.

39. (canceled)

40. The gastric residence system of claim 31, wherein the first disintegrating matrix segment comprises a time-dependent disintegrating matrix.

41. The gastric residence system of claim 31, wherein the first disintegrating matrix segment comprises:
(a) polycaprolactone (PCL), optionally wherein the first disintegrating matrix comprises about 43 wt % to about 47 wt % PCL; and/or
(b) acid terminated copolymer of DL-lactide and glycolide (50/50 molar ratio) having a viscosity midpoint of about 0.4 dl/g; optionally wherein the first disintegrating matrix comprises about 33 wt % to about 37 wt % of the acid terminated copolymer of DL-lactide and glycolide; and/or
(c) ester terminated copolymer of DL-lactide and glycolide (50/50 molar ratio) having a viscosity midpoint of about 0.4 dl/g; optionally wherein the first disintegrating matrix comprises about 15 wt % to about 20 wt % of the copolymer of DL-lactide and glycolide.
(d) polyethylene oxide, optionally wherein the polyethylene oxide has a molecular weight of about 100,000 MW (PEO 100 k), further optionally wherein the segment comprises about 1 wt % to about 3 wt % of polyethylene oxide.
(e) an optional coloring agent, optionally wherein the first disintegrating matrix comprises about 0.01 wt % to about 0.1 wt % of the coloring agent.

42-45. (canceled)

46. The gastric residence system of claim 31, wherein the first inert segment comprises:
(a) polycaprolactone (PCL), optionally wherein the first inert segment about 68 wt % to about 72 wt % PCL; and/or
(b) (BiO)₂CO₃, optionally wherein the first inert segment comprises about 28 wt % to about 32 wt % (BiO)₂CO₃.

47. The gastric residence system of claim 31, wherein the second disintegrating matrix segment comprises an enteric disintegrating matrix.

48. The gastric residence system of claim 31, wherein the second disintegrating matrix comprises:
(a) polycaprolactone (PCL), optionally wherein the second disintegrating matrix comprises about 32 wt % to about 36 wt % PCL; and/or
(b) hydroxypropyl methylcellulose acetate succinate (HPMCAS); optionally wherein the second disintegrating matrix comprises about 62 wt % to about 66 wt % HPMCAS; and/or
(c) polyethylene glycol-polypropylene glycol-polyethylene glycol (PEG-PPG-PEG) block copolymer, optionally wherein the second disintegrating matrix comprises about 1 wt % to about 3 wt % of PEG-PPG-PEG block copolymer.

49-50. (canceled)

51. The gastric residence system of claim 31, wherein the drug-eluting segment comprises:
(a) risperidone, optionally wherein the drug-eluting segments comprises about 33 wt % to about 37 wt % of risperidone; and/or
(b) polycaprolactone (PCL), optionally wherein the segment comprises about 54 wt % to about 58 wt % of PCL; and/or
(c) copovidone, optionally wherein the segment comprises about 4 wt % to about 6 wt % of copovidone; and/or
(d) polyethylene glycol-polypropylene glycol-polyethylene glycol (PEG-PPG-PEG) block copolymer, optionally wherein the segment comprises about 2 wt % to about 4 wt % of PEG-PPG-PEG block copolymer; and/or
(e) vitamin E succinate, optionally wherein the segment comprises about 0.2 wt % to about 0.8 wt % Vitamin E succinate; and/or
(f) colloidal silicon dioxide (SiO₂), optionally wherein the segment comprises about 0.2 wt % to about 0.8 wt % SiO₂; and/or
(g) an optional coloring agent, optionally wherein the segment comprises about 0.05 wt % to about 0.15 wt % of the coloring agent.

52. The gastric residence system of claim 31, wherein the proximal end of the proximal segment of the arm is attached to the central elastomer via an inert polycaprolactone (PCL) linker; optionally wherein:
(a) the proximal segment of the arm is the first inert segment, or
(b) the proximal segment of the arm is the first disintegrating matrix.

53-54. (canceled)

55. The gastric residence system of claim 31, wherein one or more segments of the arm is coated by a release-rate modulating polymer film.

56. (canceled)

57. A gastric residence system comprising:
at least three arms affixed to a central elastomer, wherein at least one arm comprises a drug-eluting segment,
each arm comprising a proximal end, a distal end, and an outer surface therebetween; wherein the proximal end of each arm is attached to the elastomer component and projects radially from the elastomer component, each arm having its distal end not attached to the elastomer component and located at a larger radial distance from the elastomer component than the proximal end;
wherein the drug eluting segment comprises a carrier polymer, and risperidone or a salt thereof;
wherein the drug eluting segment further comprises a coating comprising a release rate-modulating polymer film; and
a filament circumferentially connecting each arm.

58-81. (canceled)

82. A gastric residence system for once-weekly oral administration to a patient, comprising an extended release formulation comprising an amount of risperidone or a salt thereof, and a carrier polymer, the extended release formulation further comprising one or more excipients and a release-rate modulating polymer film;
wherein:
a) the plasma Cmax of (risperidone+9-hydroxyrisperidone) from once-weekly oral administration of the gastric residence system at steady state is less than or about equal to the plasma Cmax of (risperidone+9-hydroxyrisperidone) from once-daily oral administration of an immediate release formulation of risperidone or a salt thereof at steady state; or
b) the plasma Cavg of (risperidone+9-hydroxyrisperidone) from once-weekly oral administration of the gastric residence system at steady state is greater than or about equal to the plasma Cavg of (risperidone+9-hydroxyrisperidone) from once-daily oral administration of an immediate release formulation of risperidone or a salt thereof at steady state; or
c) the plasma concentration of (risperidone+9-hydroxyrisperidone) from once-weekly oral administration of the gastric residence system at steady state 168 hours after administration is greater than or about equal to the plasma concentration of (risperidone+9-hydroxyrisperidone) from once-daily oral administration of an immediate release formulation of risperidone or a salt thereof at steady state 24 hours after administration;
wherein the immediate release formulation comprises about one-seventh the amount of risperidone or a salt thereof by weight as compared to the amount of risperidone or a salt thereof in the gastric residence system;
or
a gastric residence system for once-weekly oral administration to a patient, comprising an extended release formulation comprising an amount of risperidone or a salt thereof, and a carrier polymer, the extended release formulation further comprising one or more excipients and a release-rate modulating polymer film:
wherein:
a1) the amount of risperidone or a salt thereof is about 14 mg, and the plasma Cmax of (risperidone+9-hydroxyrisperidone) from once-weekly oral administration of the gastric residence system at steady state is less than or equal to about 25 ng/mL: or
b1) the amount of risperidone or a salt thereof is about 14 mg, and the plasma Cavg of (risperidone+9-hydroxyrisperidone) from once-weekly oral administration of the gastric residence system at steady state is greater than or about equal to 10 ng/mL: or
c1) the amount of risperidone or a salt thereof is about 14 mg, and the plasma concentration of (risperidone+9-hydroxyrisperidone) from once-weekly oral administration of the gastric residence system at steady state 168 hours after administration is greater than or about equal to 5 ng/mL:
or
a gastric residence system for once-weekly oral administration to a patient, comprising an extended release formulation comprising an amount of risperidone or a salt thereof, and a carrier polymer, the extended release formulation further comprising one or more excipients and a release-rate modulating polymer film:
wherein:
a2) the amount of risperidone or a salt thereof is about 28 mg, and the plasma Cmax of (risperidone+9-hydroxyrisperidone) from once-weekly oral administration of the gastric residence system at steady state is less than or equal to about 50 ng/mL: or
b2) the amount of risperidone or a salt thereof is about 28 mg, and the plasma Cavg of (risperidone+9-hydroxyrisperidone) from once-weekly oral administration of the gastric residence system at steady state is greater than or about equal to 20 ng/mL: or
c2) the amount of risperidone or a salt thereof is about 28 mg, and the plasma concentration of (risperidone+9-hydroxyrisperidone) from once-weekly oral administration of the gastric residence system at steady state 168 hours after administration is greater than or about equal to 10 ng/mL:
or
a gastric residence system for once-weekly oral administration to a patient, comprising an extended release formulation comprising an amount of risperidone or a salt thereof, and a carrier polymer, the extended release formulation further comprising one or more excipients and a release-rate modulating polymer film;
wherein:
a3) the amount of risperidone or a salt thereof is about 42 mg, and the plasma Cmax of (risperidone+9-hydroxyrisperidone) from once-weekly oral administration of the gastric residence system at steady state is less than or equal to about 75 ng/mL; or
b3) the amount of risperidone or a salt thereof is about 42 mg, and the plasma Cavg of (risperidone+9-hydroxyrisperidone) from once-weekly oral administration of the gastric residence system at steady state is greater than or about equal to 30 ng/mL; or
c3) the amount of risperidone or a salt thereof is about 42 mg, and the plasma concentration of (risperidone+9-hydroxyrisperidone) from once-weekly oral administration of the gastric residence system at steady state 168 hours after administration is greater than or about equal to 15 ng/mL.
or
a gastric residence system for once-weekly oral administration to a patient, comprising an extended release formulation comprising an amount of risperidone or a salt thereof, and a carrier polymer, the extended release formulation further comprising one or more excipients and a release-rate modulating polymer film;
wherein:
a4) the amount of risperidone or a salt thereof is about 56 mg, and the plasma Cmax of (risperidone+9-hydroxyrisperidone) from once-weekly oral administration of the gastric residence system at steady state is less than or equal to about 100 ng/mL; or
b4) the amount of risperidone or a salt thereof is about 56 mg, and the plasma Cavg of (risperidone+9-hydroxyrisperidone) from once-weekly oral administration of the gastric residence system at steady state is greater than or about equal to 40 ng/mL; or
c4) the amount of risperidone or a salt thereof is about 56 mg, and the plasma concentration of (risperidone+9-hydroxyrisperidone) from once-weekly oral administration of the gastric residence system at steady state 168 hours after administration is greater than or about equal to 20 ng/mL.

83-89. (canceled)

90. A gastric residence system for once-weekly oral administration to a patient, comprising an extended release formulation comprising an amount of risperidone or a salt thereof, and a carrier polymer, the extended release formulation further comprising one or more excipients and a release-rate modulating polymer film;
wherein:
a) the plasma Cmax of (risperidone+9-hydroxyrisperidone) from once-weekly oral administration of the gastric residence system at steady state is less than or about equal to the plasma Cmax of (risperidone+9-hydroxyrisperidone) from once-daily oral administration of an immediate release formulation of risperidone or a salt thereof at steady state; or
b) the plasma Cavg of (risperidone+9-hydroxyrisperidone) from once-weekly oral administration of the gastric residence system at steady state is greater than or about equal to the plasma Cavg of (risperidone+9-hydroxyrisperidone) from once-daily oral administration of an immediate release formulation of risperidone or a salt thereof at steady state; or
c) the plasma concentration of (risperidone+9-hydroxyrisperidone) from once-weekly oral administration of the gastric residence system at steady state 168 hours after administration is greater than or about equal to the plasma concentration of (risperidone+9-hydroxyrisperidone) from once-daily oral administration of an immediate release formulation of risperidone or a salt thereof at steady state 24 hours after administration;
wherein the immediate release formulation comprises about one-eighth the amount of risperidone or a salt thereof by weight as compared to the amount of risperidone or a salt thereof in the gastric residence system,
or
a gastric residence system for once-weekly oral administration to a patient, comprising an extended release formulation comprising an amount of risperidone or a salt thereof, and a carrier polymer, the extended release formulation further comprising one or more excipients and a release-rate modulating polymer film;
wherein:
a1) the amount of risperidone or a salt thereof is about 16 mg, and the plasma Cmax of (risperidone+9-hydroxyrisperidone) from once-weekly oral administration of the gastric residence system at steady state is less than or equal to about 25 ng/mL; or
b1) the amount of risperidone or a salt thereof is about 16 mg, and the plasma Cavg of (risperidone+9-hydroxyrisperidone) from once-weekly oral administration of the gastric residence system at steady state is greater than or about equal to 10 ng/mL; or
c1) the amount of risperidone or a salt thereof is about 16 mg, and the plasma concentration of (risperidone+9-hydroxyrisperidone) from once-weekly oral administration of the gastric residence system at steady state 168 hours after administration is greater than or about equal to 5 ng/mL;
or
a gastric residence system for once-weekly oral administration to a patient, comprising an extended release formulation comprising an amount of risperidone or a salt thereof, and a carrier polymer, the extended release formulation further comprising one or more excipients and a release-rate modulating polymer film;
wherein:
a2) the amount of risperidone or a salt thereof is about 32 mg, and the plasma Cmax of (risperidone+9-hydroxyrisperidone) from once-weekly oral administration of the gastric residence system at steady state is less than or equal to about 50 ng/mL; or
b2) the amount of risperidone or a salt thereof is about 32 mg, and the plasma Cavg of (risperidone+9-hydroxyrisperidone) from once-weekly oral administration of the gastric residence system at steady state is greater than or about equal to 20 ng/mL; or
c2) the amount of risperidone or a salt thereof is about 32 mg, and the plasma concentration of (risperidone+9-hydroxyrisperidone) from once-weekly oral administration of the gastric residence system at steady state 168 hours after administration is greater than or about equal to 10 ng/mL;
or
a gastric residence system for once-weekly oral administration to a patient, comprising an extended release formulation comprising an amount of risperidone or a salt thereof, and a carrier polymer, the extended release formulation further comprising one or more excipients and a release-rate modulating polymer film:
wherein:
a3) the amount of risperidone or a salt thereof is about 48 mg, and the plasma Cmax of (risperidone+9-hydroxyrisperidone) from once-weekly oral administration of the gastric residence system at steady state is less than or equal to about 75 ng/mL: or
b3) the amount of risperidone or a salt thereof is about 48 mg, and the plasma Cavg of (risperidone+9-hydroxyrisperidone) from once-weekly oral administration of the gastric residence system at steady state is greater than or about equal to 30 ng/mL: or
c3) the amount of risperidone or a salt thereof is about 48 mg, and the plasma concentration of (risperidone+9-hydroxyrisperidone) from once-weekly oral administration of the gastric residence system at steady state 168 hours after administration is greater than or about equal to 15 ng/mL:
or
a gastric residence system for once-weekly oral administration to a patient, comprising an extended release formulation comprising an amount of risperidone or a salt thereof, and a carrier polymer, the extended release formulation further comprising one or more excipients and a release-rate modulating polymer film:
wherein:
a4) the amount of risperidone or a salt thereof is about 64 mg, and the plasma Cmax of (risperidone+9-hydroxyrisperidone) from once-weekly oral administration of the gastric residence system at steady state is less than or equal to about 100 ng/mL; or
b4) the amount of risperidone or a salt thereof is about 64 mg, and the plasma Cavg of (risperidone+9-hydroxyrisperidone) from once-weekly oral administration of the gastric residence system at steady state is greater than or about equal to 40 ng/mL: or
c4) the amount of risperidone or a salt thereof is about 64 mg, and the plasma concentration of (risperidone+9-hydroxyrisperidone) from once-weekly oral administration of the gastric residence system at steady state 168 hours after administration is greater than or about equal to 20 ng/mL.

91-101. (canceled)
